(19) United States
(12) Patent Application Publication
(10) Pub. No.: US 2024/0122932 A1
(43) Pub. Date: Apr. 18, 2024

(54) Methods of Treating B-Cell Malignancy Using Bcl-2 Inhibitor

(71) Applicant: BeiGene Switzerland GmbH, Basel (CH)

(72) Inventors: James D. Hilger, San Mateo, CA (US); Jane Huang, San Mateo, CA (US); David Simpson, Auckland (NZ)

(73) Assignee: BeiGene Switzerland GmbH, Basel (CH)

(21) Appl. No.: 18/524,170

(22) Filed: Nov. 30, 2023

Related U.S. Application Data
(63) Continuation of application No. PCT/US2022/031903, filed on Jun. 2, 2022.
(60) Provisional application No. 63/340,642, filed on May 11, 2022.
(60) Provisional application No. 63/195,892, filed on Jun. 2, 2021.

(51) Int. Cl. A61K 31/519; A61P 35/00

(52) U.S. Cl. CPC A61K 31/519; A61P 35/00

(57) ABSTRACT

The present disclosure provides methods of treating B-cell malignancy in a subject with a Bcl-2 inhibitor, in particularly 2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide or a pharmaceutically acceptable salt thereof, or its combination with a Bruton's tyrosine kinase (BTK) inhibitor, particularly (S)-7-(1-acryloylpiperidin-4-yl)-2-(4-phenoxyphenyl)-4,5,6,7-tetrahydropyrazolo-[1,5-a]pyrimidine-3-carboxamide or a pharmaceutically acceptable salt thereof.

### CROSS-REFERENCE TO RELATED APPLICATIONS

This application is a continuation application of International Application No. PCT/US2022/031903, filed Jun. 2, 2022, which claims priority to, and the benefit of US Application Nos. 63/340,642 filed on May 11, 2022 and 63/195,892 filed on Jun. 2, 2021, the disclosures of which are hereby incorporated by reference in their entireties for all purposes.

### FIELD OF THE DISCLOSURE

Disclosed herein are methods of treating B-cell malignancy with a Bcl-2 inhibitor, in particularly 2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide or a pharmaceutically acceptable salt thereof, or its combination with a Bruton's tyrosine kinase (BTK) inhibitor, particularly (S)-7-(1-acryloylpiperidin-4-yl)-2-(4-phenoxyphenyl)-4,5,6,7-tetrahydropyrazolo-[1,5-a]pyrimidine-3-carboxamide or a pharmaceutically acceptable salt thereof.

### BACKGROUND OF THE DISCLOSURE

Impaired apoptosis plays a central role in tumor development, tumor maintenance, and therapeutic resistance. Apoptosis can be triggered via two main pathways: the extrinsic or death-receptor-mediated pathway, and the intrinsic or mitochondria) pathway (Czabotar et al 2014). It is the intrinsic pathway that is more commonly perturbed in lymphoid malignancies. Cell death mediated through this pathway is regulated by members of a family of proteins related to B-cell lymphoma-2 (Bcl-2), which is considered to contain three subfamilies. The pro-survival subgroup (Bcl-2, Bcl-xL, Bcl-W, Mcl-1, A1/Bfl-1, and possibly Bcl-B) promotes cell survival by inhibiting their pro-apoptotic relatives. The pro-apoptotic BAX/BAK-like proteins, including BOK, are the essential effectors of apoptosis, and the BH3-only proteins (BIM, PUMA, BID, NOXA, BMF, BIK, and HRK) are the initiators of apoptosis (Anderson et al 2014). In healthy cells, the pro-survival Bcl-2 proteins bind and inhibit BAX and BAK after they have been partially activated, impairing the ability of BAX/BAK to oligomerize and form pores to induce mitochondrial outer membrane permeabilization. The BH3-only proteins are induced transcriptionally or post-transcriptionally in response to diverse stresses and initiate apoptosis by either binding the pro-survival Bcl-2 proteins, thereby unleashing BAX/BAK, or by directly activating these effectors of apoptosis. The various Bcl-2 family proteins have differential specificity of binding to one another, resulting in a complex but ordered network of interactions governing cell fate (Roberts 2016).

Bcl-2 was the first anti-apoptotic protein discovered in 1980s as a consequence of t(14.18) chromosomal translocation and the hallmark of FL. BCL-2 gene resides on chromosome 18q21.33. The Bcl-2 protein has 239 amino acids and a molecular weight of 26 kDa (Schenk et al 2017). Bcl-2 is widely expressed during development and becomes restricted upon maturation in many tissues (Kondo et al 2008). Mice lacking Bcl-2 succumb to polycystic kidney disease early in life because Bcl-2 is critical for the survival of renal epithelial progenitor cells during embryogenesis (Veis et al 1993). The Bcl-2-deficient mice also have abnormally reduced numbers of mature, resting B and T lymphocytes, and gray prematurely because of the aberrant death of melanocytes (Veis et al 1993, Yamamura et al 1996). Although originally believed to act as a classical growth-driving oncogene, it was later shown that Bcl-2 instead promotes malignant cell survival by attenuating apoptosis. Transgenic mice with pan-hematopoietic Bcl-2 expression (VavP-BCL-2) preferentially develop follicular lymphoma, preceded by florid germinal center hyperplasia (Egle et al 2004). Mice co-expressing BCL-2 and MYC transgenes developed lymphomas markedly faster than littermates expressing either transgene alone, validating BCL-2 as an oncogene (Adams and Cory 2007).

High Bcl-2 expression is almost universal in CLL, FL, MCL, and Waldenstrom macroglobulinemia (WM); in contrast, the levels of Bcl-2 expression are somewhat more variable among multiple myeloma (MM) and substantially more variable among DLBCL and B-lineage acute lymphoblastic leukemia (Roberts and Huang 2017). When Bcl-2 is overexpressed, the ratio of pro- and anti-apoptotic Bcl-2 family members is disturbed and apoptotic cell death can be prevented. Moreover, Bcl-2 protein is closely related to chemoresistance in hematological tumors. As Bcl-2-mediated resistance to intrinsic apoptosis is considered as a key to pathogenesis, targeting Bcl-2 can improve apoptosis and overcome drug resistance to cancer therapy. Thus, Bcl-2 has become an attractive target for therapeutic strategy in cancer.

Venetoclax (ABT-199) was approved for treating patients with chronic lymphocytic leukemia (CLL) and acute myeloblastic leukemia (AML). However, despite this high clinical activity and favorable safety profile, patients can develop acquired resistance to venetoclax overtime with continuous treatment. Blombery et al demonstrated that the Gly101Val mutation (G101V mutation) in BCL-2 confers acquired refractoriness by reducing the binding affinity of venetoclax without disrupting the binding of pro-apoptotic proteins to Bcl-2. The novel Gly101Val mutation in Bcl-2 was identified at progression in 7 of 15 patients. This mutation is mainly found in patients after long-term exposure to venetoclax monotherapy (Tausch et al 2019).

WO2019/210828A disclosed a series of compounds having the following Formulas (III-B), (III-C), (III-D) or (III-E), or a stereoisomer thereof, or a pharmaceutically acceptable salt thereof, as Bcl-2 inhibitors.

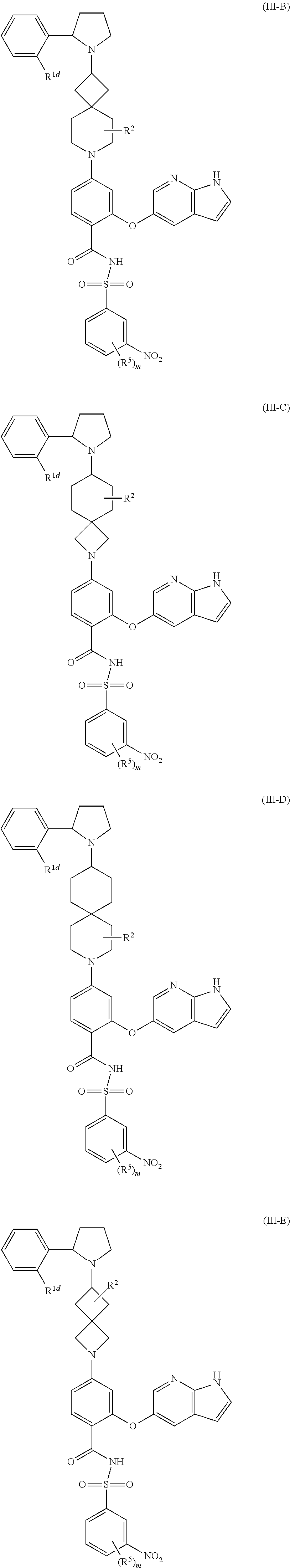

The compounds disclosed in WO2019/210828A are potent and selective Bcl-2 protein inhibitors.

### SUMMARY OF THE DISCLOSURE

The inventors of the present disclosure have found that a Bcl2 inhibitor having Formulas (III-B), (III-C), (III-D) or (III-E), in particularly 2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide (Compound 1) or a pharmaceutically acceptable salt thereof, exhibited potent cell killing activity against a variety of lymphoma and leukemia cell lines, including MV4-11 (acute myeloid leukemias, AML), OCI-LY10 (B-cell non-Hodgkin's lymphoma, B-NHL), Toledo (diffuse large B-cell lymphomas. DLBCL), DOHH2 (follicular lymphomas, FL), DHL-4 (Germinal center B-cell like diffuse large B-cell lymphomas, GCB-DLBCL) and MAVER-1 (mantle cell lymphomas, MCL). The IC₅₀ values were found ranging from 0.6 nM to 13 nM.

The inventors of the present disclosure have also found that a Bcl2 inhibitor having Formulas (III-B), (III-C), (III-D) or (III-E), in particularly Compound 1 or a pharmaceutically acceptable salt thereof, demonstrated significant inhibition of tumor growth in cancer with high safety, including B-cell malignancies selected from non-Hodgkin lymphoma (NHL) expected to be at low risk of tumor lysis syndrome, low-tumor-burden chronic lymphocytic leukemia/small lymphocytic lymphoma (CLL/SLL), high-tumor-burden CLL/SLL, mantle cell lymphoma (MCL), Waldenström macroglobulinemia (WM) or acute lymphoblastic leukemia (ALL).

In addition, the inventors of the present disclosure have found that a Bcl2 inhibitor having Formulas (III-B), (III-C), (III-D) or (III-E), in particularly Compound 1 or a pharmaceutically acceptable salt thereof, in combination with a BTK inhibitor disclosed in WO2014/173289A, particularly (S)-7-(1-acryloylpiperidin-4-yl)-2-(4-phenoxyphenyl)-4,5,6,7-tetra-hydropyrazolo[1,5-a]pyrimidine-3-carboxamide (Zanubrutinib, Compound B) produces significant inhibition of tumor growth in cancers as compared with the efficacy of each therapeutic as a single agent. Further, the combination therapy demonstrated significant inhibition of tumor growth in cancer with high safety, including B-cell malignancies selected from chronic lymphocytic leukemia/small lymphocytic lymphoma (CLL/SLL), or mantle cell lymphoma (MCL).

In a first aspect, disclosed herein is a method of treating B-cell malignancies with a Bcl-2 inhibitor, wherein the Bcl-2 inhibitor is a compound represented by the following Formulas (III-B), (III-C), (III-D) or (III-E).

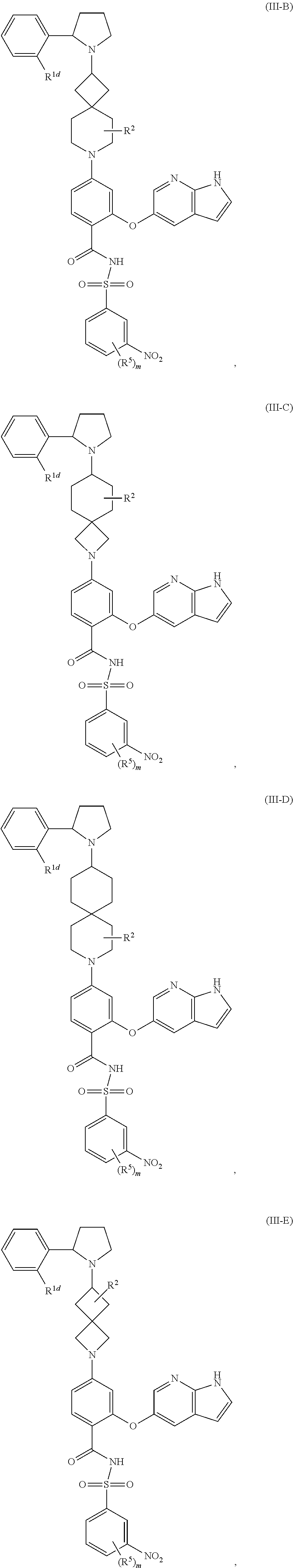

or a pharmaceutically acceptable salt thereof, or a stereoisomer thereof.
wherein,
R², at each occurrence, is independently selected from the group consisting of hydrogen, halogen, and —C₁₋₈alkyl optionally substituted with halogen;
R^{1d}, at each occurrence, is independently halogen, —C₁₋₈alkyl, —C₂₋₈alkenyl, —C₂₋₈alkynyl, cycloalkyl, heterocyclyl, aryl, heteroaryl, oxo, —CN, —NO₂, —OR^{Ba}, —SO₂R^{Ba}, —COR^{Ba}, —CO₂R^{Ba}, —CONR^{Ba}R^{Bb}, —C(═NR^{Ba})NR^{Bb}R^{Bc}, —NR^{Ba}R^{Bb}, —NR^{Ba}COR^{Bb}, —NR^{Ba}CONR^{Bb}R^{Bc}, —NR^{Ba}CO₂R^{Bb}, —NR^{Ba}SONR^{Bb}R^{Bc}, —NR^{Ba}SO₂NR^{Bb}R^{Bc}, or —NR^{Ba}SO₂R^{Bb}; wherein said —C₁₋₈alkyl, —C₂₋₈alkenyl, —C₂₋₈alkenyl, cycloalkyl, heterocyclyl, aryl or heteroaryl are each independently optionally substituted with 1 to 4 substituents R^{Bd};
R^{Ba}, R^{Bb}, and R^{Bc}, are each independently hydrogen, —C₁₋₈alkyl, —C₂₋₈alkenyl, —C₂₋₈alkenyl, cycloalkyl, heterocyclyl, aryl, or heteroaryl, each of said —C₁₋₈alkyl, —C₂₋₈alkenyl, —C₂₋₈alkynyl, cycloalkyl, heterocyclyl, aryl, or heteroaryl is optionally substituted with halogen, hydroxy, —NH₂ or —N(C₁₋₆alkyl)₂, —C₁₋₈alkyoxy, cycloalkyl, heterocyclyl, aryl, or heteroaryl;
R^{Bd}, at each occurrence, is independently hydrogen, halogen, oxo, —CN, —NO₂, —C₁₋₈alkyl, —C₂₋₈alkenyl, —C₂₋₈alkynyl, cycloalkyl, heterocyclyl, aryl, or heteroaryl, each of said —C₁₋₈alkyl, —C₂₋₈alkenyl, —C₂₋₈alkynyl, cycloalkyl, heterocyclyl, aryl, or heteroaryl is optionally substituted with halogen, hydroxy, —C₁₋₈alkyoxy, cycloalkyl, heterocyclyl, aryl, or heteroaryl;
m is an integer of 1-4;
R⁵ is -L⁵-CyC,
   Wherein L⁵ is a direct bond, —(CR^{a}R^{b})ₜ—, —(CR^{a}R^{b})ₜ₋₁—(CR^{c}═CR^{d})—(CR^{a}R^{b})ᵥ₋₁—, —(CR^{a}R^{b})ₜ₋₁—(C≡C)—(CR^{a}R^{b})ᵥ₋₁—, —O—, —S—, —S(O)—, —SO₂—, —C(O)—, C(O)O—, —OC(O)—, —NR^{a}—, —C(O)NR^{a}—, —NR^{a}C(O)—, —NR^{a}C(O)O—, —NR^{a}C(O)NR^{b}—, —SO₂NR^{a}—, —NR^{a}SO₂—, —NR'S(O)₂NR^{b}—, —NR^{a}S(O)NR^{b}—, —C(O)NR^{a}SO₂—, —C(O)NR^{a}SO—, or —C(═NR^{a})NR^{b}—, wherein t and v, at each occurrence, are independently a number of 1 to 7, and one or two CR^{a}R^{b} moieties in —(CR^{a}R^{b})—, —(CR^{a}R^{b})^{t-1}—(CR^{c}═CR^{d})—(CR^{a}R^{b})ᵥ₋₁—, —(CR^{a}R^{b})ₜ₋₁—(C≡C)—(CR^{a}R^{b})ᵥ₋₁— are un-replaced or replaced with one or more moieties selected from O, S, SO, SO₂, C(O) or NR^{a};
   CyC is cycloalkyl, heterocyclyl, aryl, or heteroaryl, each of which is optionally substituted with one or two substituents R^{5a};
   R^{5a}, at each occurrence, is independently selected from hydrogen, halogen, cyan, oxo, —NO₂, —OR^{5b}, —SR^{5b}, —NR^{5b}R^{5c}, —COR^{5b}, —SO₂R^{5b}, —C(═O)OR^{5b}, —C(═O)NR^{5b}R^{5c}, —C(═NR^{5b})NR^{5c}R^{5d}, —N(R^{5b})C(═O)R^{5c}, —N(R^{5b})C(═O)OR^{5c}, —N(R^{5b})C(O)NR^{5c}R^{5d}, —N(R^{5b})S(O)NR^{5c}R^{5d}, —N(R^{5b})S(O)₂NR^{5c}R^{5d}, —NR^{5b}SO₂R^{5c}, —C₁₋₈alkyl, —C₂₋₈alkenyl, —C₂₋₈alkynyl, -cycloalkyl, heterocyclyl, aryl, or heteroaryl, each of said —C₁₋₈alkyl, —C₂₋₈alkenyl, —C₂₋₈alkynyl, -cycloalkyl, heterocyclyl, aryl, or heteroaryl is optionally substituted with one or two substituents R^{5c};
      wherein R^{5b}, R^{5c}, and R^{5d} are each independently hydrogen, —C₁₋₈alkyl, —C₂₋₈alkenyl, —C₂₋₈alkynyl, cycloalkyl, heterocyclyl, aryl, or heteroaryl, each of said —C₁₋₈alkyl, —C₂₋₈alkenyl, C₂₋₈alkynyl, -cycloalkyl, heterocyclyl, aryl, or heteroaryl is optionally substituted with one or two substituents R^{5e};
      R^{5e}, at each occurrence, is independently selected from hydrogen, halogen, cyano, oxo, —NO₂, —OR^{5f}, —SR^{5f}, —NR^{5f}R^{5g}, —COR^{5f}, —SO₂R^{5f}, —C(═O)OR^{5f}, —C(═O)NR^{5f}R^{5g}, —C(═NR^{5f})NR^{5g}R^{5h}, —N(R^{5f})C(═O)R^{5g}, —N(R^{5f})C(═O)OR^{5g}, —N(R^{5f})C(O)NR^{5g}R^{5h}, —N(R^{5f})S(O)NR^{5g}R^{5h}, —N(R^{5f})S(O)₂NR^{5g}R^{5h}, —NR^{5f}SO₂R^{5g}, —C₁₋₈alkyl, —C₂₋₈alkenyl, —C₂₋₈alkynyl, -cycloalkyl, heterocyclyl, aryl, or heteroaryl;
         R^{5f}, R^{5g}, and R^{5h} are each independently hydrogen, —C₁₋₈alkyl, —C₂₋₈alkenyl, —C₂₋₈alkynyl, cycloalkyl, heterocyclyl, aryl, or heteroaryl;
   or, two adjacent R⁵ on the phenyl ring together with the phenyl ring form a benzo ring, said ring is optionally substituted with halogen, oxo, cyano, —NO₂, —OR⁵ⁱ, —SR⁵ⁱ, —NR⁵ⁱR^{5j}, —COR⁵ⁱ, —SO₂R⁵ⁱ, —C(═O)OR⁵ⁱ, —C(═O)NR⁵ⁱR^{5j}, —C(═NR⁵ⁱ)NR^{5j}R^{5k}, —N(R⁵ⁱ)C(═O)R^{5j}, —N(R⁵ⁱ)C(═O)OR^{5j}, —N(R⁵ⁱ)C(O)NR^{5j}R^{5k}, —N(R⁵ⁱ)S(O)NR^{5j}R^{5k}, —N(R⁵ⁱ)S(O)₂NR^{5j}R^{5k}, —NR⁵ⁱSO₂R^{Sk}, —C₁₋₈alkyl, —C₂₋₈alkenyl, —C₂₋₈alkynyl, -cycloalkyl, heterocyclyl, aryl, or heteroaryl;
      R⁵ⁱ, R^{5j}, and R^{5k} are independently hydrogen, —C₁₋₈alkyl, —C₂₋₈alkenyl, —C₂₋₈alkynyl, cycloalkyl, heterocyclyl, aryl, or heteroaryl, each of said —C₁₋₈alkyl, —C₂₋₈alkenyl, —C₂₋₈alkynyl, cycloalkyl, heterocyclyl, aryl, or heteroaryl is optionally substituted with halogen, hydroxy or —C₁₋₈alkyoxy;
   R^{a}, R^{b}, R^{c}, and R^{d} at each occurrence, are independently hydrogen, —C₁₋₈alkyl, —C₂₋₈alkenyl, —C₂₋₈alkynyl, cycloalkyl, heterocyclyl, aryl, or heteroaryl, said —C₁₋₈alkyl, —C₂₋₈alkenyl, —C₂₋₈alkynyl, cycloalkyl, heterocyclyl, aryl, or heteroaryl are each independently substituted with —CN, halogen, —NO₂, —NR^{e}R^{f}, oxo, —OR^{e}, or —SR^{e}; and
      wherein R^{e} and R^{f} are each independently hydrogen, C₁₋₈alkyl, C₁₋₈alkoxy-C₁₋₈alkyl-, C₂₋₈alkenyl, C₂₋₈alkynyl, cycloalkyl, aryl, heterocyclyl, or heteroaryl.

In a second aspect, disclosed herein is a Bcl-2 inhibitor of Formulas (III-B), (III-C), (Ill-D) or (III-E) or a stereoisomer thereof, or a pharmaceutically acceptable salt thereof, for use in the treatment of B-cell malignancies.

In a third aspect, disclosed herein is a method of treating B-cell malignancies in a subject, said method comprising administering to the subject a therapeutically effective amount of a Bcl-2 inhibitor of Formulas (III-B), (III-C), (III-D) or (III-E) or a stereoisomer thereof, or a pharmaceutically acceptable salt thereof.

In a fourth aspect, disclosed herein is a method of treating B-cell malignancies in a subject, comprising administering to the subject in need thereof a therapeutically effective amount of a Bcl-2 inhibitor, or a stereoisomer thereof, or a pharmaceutically acceptable salt thereof, in combination with a therapeutically effective amount of (S)-7-(1-acryloylpiperidin-4-yl)-2-(4-phenoxyphenyl)-4,5,6,7-tetra-hydropyrazolo[1,5-a]pyrimidine-3-carboxamide (Compound B) or a pharmaceutically acceptable salt thereof.

In a fifth aspect, disclosed herein is a use of a pharmaceutical composition in the manufacture of a medicament for use in the treatment of B-cell malignancies, said pharmaceutical combination comprising a Bcl-2 inhibitor of Formulas (III-B), (III-C), (III-D) or (III-E) or a stereoisomer thereof, or a pharmaceutically acceptable salt thereof.

In a sixth aspect, disclosed herein is a use of a pharmaceutical combination in the manufacture of a medicament for use in the treatment of cancer, said pharmaceutical combination comprising a Bcl-2 inhibitor, or a stereoisomer thereof, or a pharmaceutically acceptable salt thereof, and (S)-7-(1-acryloylpiperidin-4-yl)-2-(4-phenoxyphenyl)-4,5,6,7-tetra-hydropyrazolo[1,5-a]pyrimidine-3-carboxamide (Compound B, Zanubrutinib), or a pharmaceutically acceptable salt thereof.

In an embodiment of each of the above aspects, the Bcl-2 inhibitor is 2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide (Compound 1) or a pharmaceutically acceptable salt thereof.

In one embodiment of each of the above aspects, the B-cell malignancies are relapsed/refractory.

In one embodiment of each of the above aspects, the B-cell malignancy is B-cell malignancies selected from non-Hodgkin lymphoma (NHL), chronic lymphocytic leukemia/small lymphocytic lymphoma (CLL/SLL), mantle cell lymphoma (MCL), Waldenström macroglobulinemia (WM) or acute lymphoblastic leukemia (ALL).

In one preferred embodiment of each of the above aspects, the B-cell malignancy is non-Hodgkin lymphoma (NHL) selected from follicular lymphoma (FL), diffuse large B-cell lymphoma (DLBL), marginal zone lymphoma (MZL) or transformed NHL.

In one preferred embodiment of each of the above aspects, the B-cell malignancy is low-tumor-burden chronic lymphocytic leukemia/small lymphocytic lymphoma (CLL/SLL) or high-tumor-burden CLL/SLL.

In one preferred embodiment of each of the above aspects, the B-cell malignancy is mantle cell lymphoma (MCL).

In one preferred embodiment of each of the above aspects, the B-cell malignancy is Waldenström macroglobulinemia (WM).

In one embodiment of each of the above aspects, the Bcl-2 inhibitor is orally administrated at a dose of 1 mg once daily (QD) to 640 mg QD, or 20 mg QD to 640 mg QD, according to a dose ramp-up schedule.

In one embodiment of each of the above aspects, the Bcl-2 inhibitor is orally administrated at a dose according to a daily ramp-up schedule. Preferably, the daily ramp-up schedule comprises the first dose at day 1, the second dose at day 2, and a recommended dose at day 3 and beyond, wherein the second dose at day 3 and beyond is higher than the second dose at day 2, and the second dose at day 2 is higher than the first dose at day 1. In some more preferred embodiment, the recommended dose is 40 mg, 80 mg, 160 mg, 320 mg, or 640 mg daily, and the Bcl-2 inhibitor is orally administrated at daily ramp-up schedule comprising the first dose at day 1 at 25% of the recommended dose, the second dose at day 2 at 50% of recommended dose, and the daily dose at day 3 and beyond at 100% of the recommended dose. In some more preferred embodiment, the first dose at day 1 is about 10-160 mg/day, the second dose at day 2 is about 20-320 mg/day, and the daily dose at day 3 and beyond is about 40-640 mg/day. In some more preferred embodiment, the first dose at day 1 is about 10, 20, 40, 80 or 160 mg/day, the second dose at day 2 is about 20, 40, 80, 160 or 320 mg/day, and the daily dose at day 3 and beyond is about 40 mg, 80 mg, 160 mg, 320 mg, or 640 mg daily. In particular, the first dose at day 1 is about 160 mg/day, the second dose at day 2 is about 320 mg/day, and the daily dose at day 3 and beyond is about 640 mg daily. In some embodiment, the period of daily ramp-up schedule administration lasts for two days. In some embodiment, the period of administration lasts for three days or more. In some embodiment, B-cell malignancy is at lower risk of TLS. In some embodiment, the B-cell malignancy is NHLs (excluding MCL). In some preferred embodiment, the B-cell malignancy is FL, DLBCL, MZL or transformed NHL.

In one embodiment of each of the above aspects, the Bcl-2 inhibitor is orally administrated at a dose according to a weekly ramp-up schedule. Preferably, the weekly ramp-up schedule comprises the first dose at week 1, the second dose at week 2, the third dose at week 3, the fourth dose at week 4, the fifth dose at week 5, a subsequently weekly ramp-up schedule, and a recommended dose at a certain week and beyond, wherein the dose at the subsequent week is at least double of the dose the previous week until the weekly recommended dose has been met and the subsequently weekly ramp-up schedule is a weekly ramp-up dosing schedule for 0, 1, 2, 3, or 4 weeks.

In some embodiment, the Bcl-2 inhibitor is orally administrated at a dose according to a weekly ramp-up schedule beginning with 1 mg daily at week 1. In some more preferred embodiment, the recommended dose is 40 mg, 80 mg, 160 mg, 320 mg, or 640 mg daily, and the Bcl-2 inhibitor is orally administrated by weekly ramp-up schedule comprising a dose steps of 1 mg, 2 mg, 5 mg, 10 mg, 20 mg, 40 mg, 80 mg, 160 mg, 320 mg or 640 mg daily. In some embodiment, the first dose at week 1 is about 1 mg/day, the second dose at week 2 is about 2 mg/day, the third dose at week 3 is about 5 mg/day, the fourth dose at week 4 is about 10 mg/day, the fifth dose at week 5 is about 20 mg/day, the sixth dose at week 6 is about 40 mg/day, and the seventh dose at week 7 and beyond is about 80 mg/day. In some embodiment, the first dose at week 1 is about 1 mg/day, the second dose at week 2 is about 2 mg/day, the third dose at week 3 is about 5 mg/day, the fourth dose at week 4 is about 10 mg/day, the fifth dose at week 5 is about 20 mg/day, the sixth dose at week 6 is about 40 mg/day, the seventh dose at week 7 is about 80 mg/day, and the eighth dose at week 8 and beyond is about 160 mg/day. In some embodiment, the first dose at week 1 is about 1 mg/day, the second dose at week 2 is about 2 mg/day, the third dose at week 3 is about 5 mg/day, the fourth dose at week 4 is about 10 mg/day, the fifth dose at week 5 is about 20 mg/day, the sixth dose at week 6 is about 40 mg/day, the seventh dose at week 7 is about 80 mg/day, the eighth dose at week 8 is about 160 mg/day, and the ninth dose at week 9 and beyond is about 320 mg/day. In some embodiment, the first dose at week 1 is about 1 mg/day, the second dose at week 2 is about 2 mg/day, the third dose at week 3 is about 5 mg/day, the fourth dose at week 4 is about 10 mg/day, the fifth dose at week 5 is about 20 mg/day, the sixth dose at week 6 is about 40 mg/day, the seventh dose at week 7 is about 80 mg/day, the eighth dose at week 8 is about 160 mg/day, the ninth dose at week 9 is about 320 mg/day, and the tenth dose at week 10 beyond is about 640 mg/day. In some embodiment, the period of weekly ramp-up schedule administration lasts for five, six, seven, eight or nine weeks. In some embodiments, the period of administration lasts for six, seven, eight, nine, or ten weeks or more. In some embodiments, the B-cell malignancy, selected from CLL/SLL, MCL or WM. In some embodiment, the B-cell malignancy, selected from CLL/SLL with low-tumor-burden, CLL/SLL with high-tumor-burden, or CLL/SLL with prior venetoclax treatment, MCL or WM.

In one embodiment of the above aspects, (S)-7-(1-acryloylpiperidin-4-yl)-2-(4-phenoxyphenyl)-4,5,6,7-tetra-hydropyrazolo[1,5-a]pyrimidine-3-carboxamide (Compound B) is orally administrated at a dose 320 mg/day (160 mg twice daily or 320 mg once daily), and the Bcl-2 inhibitor is orally administrated by weekly ramp-up schedule. Preferably, Compound B is orally administrated beginning 8-12 weeks before Compound 1 is administrated. Preferably, the weekly ramp-up schedule comprises the first dose at week 1, the second dose at week 2, the third dose at week 3, the fourth dose at week 4, the fifth dose at week 5, a subsequently weekly ramp-up schedule, and a recommended dose at a certain week and beyond, wherein the dose at the subsequent week is at least double of the dose the previous week until the weekly recommended dose has been met, and the subsequently weekly ramp-up schedule is a weekly ramp-up dosing schedule for 0, 1, 2, 3, or 4 weeks.

In some embodiment, the Bcl-2 inhibitor is orally administrated at a dose according to a weekly ramp-up schedule beginning with 1 mg daily at week 1. In some more preferred embodiment, the recommended dose is 40 mg, 80 mg, 160 mg, 320 mg, or 640 mg daily, and the Bcl-2 inhibitor is orally administrated by weekly ramp-up schedule comprising a dose steps of 1 mg, 2 mg, 5 mg, 10 mg, 20 mg, 40 mg, 80 mg, 160 mg, 320 mg or 640 mg daily. In some embodiment, the first dose at week 1 is about 1 mg/day, the second dose at week 2 is about 2 mg/day, the third dose at week 3 is about 5 mg/day, the fourth dose at week 4 is about 10 mg/day, the fifth dose at week 5 is about 20 mg/day, the sixth dose at week 6 is about 40 mg/day, and the seventh dose at week 7 and beyond is about 80 mg/day. In some embodiment, the first dose at week 1 is about 1 mg/day, the second dose at week 2 is about 2 mg/day the third dose at week 3 is about 5 mg/day, the fourth dose at week 4 is about 10 mg/day, the fifth dose at week 5 is about 20 mg/day, the sixth dose at week 6 is about 40 mg/day, the seventh dose at week 7 is about 80 mg/day, and the eighth dose at week 8 and beyond is about 160 mg/day. In some embodiment, the first dose at week 1 is about 1 mg/day, the second dose at week 2 is about 2 mg/day, the third dose at week 3 is about 5 mg/day, the fourth dose at week 4 is about 10 mg/day, the fifth dose at week 5 is about 20 mg/day, the sixth dose at week 6 is about 40 mg/day, the seventh dose at week 7 is about 80 mg/day, the eighth dose at week 8 is about 160 mg/day, and the ninth dose at week 9 and beyond is about 320 mg/day. In some embodiment, the first dose at week 1 is about 1 mg/day, the second dose at week 2 is about 2 mg/day, the third dose at week 3 is about 5 mg/day, the fourth dose at week 4 is about 10 mg/day, the fifth dose at week 5 is about 20 mg/day, the sixth dose at week 6 is about 40 mg/day, the seventh dose at week 7 is about 80 mg/day, the eighth dose at week 8 is about 160 mg/day, the ninth dose at week 9 is about 320 mg/day, and the tenth dose at week 10 beyond is about 640 mg/day. In some embodiment, the period of weekly ramp-up schedule administration lasts for five, six, seven, eight or nine weeks. In some embodiment, the period of administration lasts for six, seven, eight, nine, or ten weeks or more. In some embodiment, the B-cell malignancy is CLL/SLL including R/R CLL/SLL or naïve CLL/SLL, or MCL.

In the monotherapy, among patients with R/R NHL, significant reduction in SPD from baseline were seen in most patients, two of 20 (10%) patients have responded including 1 PR at 160 mg and 1 CR at 320 mg, and 23 patients were off treatment due to progressive disease (n=20), adverse events (n=1) and other or physician decision (n=2); among patients with R/R WM, one of 2 (50%) have achieved a minor response at 80 mg. In the combination therapy, among patients with R/R MCL, five of 10 (50%) patients have achieved PR or better at either 80 or 100 mg including 1 CR at each dose level, and 1 R/R MCL was off treatment due to progressive disease. Further, among patients with CLL/SLL in monotherapy and combination therapy, significant reduction in absolute lymphocyte count (ALC) was noted among all patients with CLL during ramp-up, with reduction in lymphocytes noted at dose levels as low as 1 mg. In the monotherapy, four of 6 (67%) patients have achieved partial response with lymphocytosis (PR-L) or better at either 80 or 160 mg of Compound 1. In the combination therapy, sixteen of 20 (80%) patients with R/R CLL/SLL have achieved PR-L or better across dose levels ranging between 40-320 mg, and 1 patient with R/R CLL/SLL were off treatment due to progressive disease.

The results of 78 patients suggest that Compound 1 is tolerable in patients with CLL or NHL at the dose levels tested. Dose escalation concluded for monotherapy patients with NHL with only 1 DLT seen and no MTD reached, and only 1 DLT was seen amongst monotherapy patients with CLL. Grade ≥3 AEs have been infrequent and manageable.

Findings suggest that the combination of Compound 1 and zanubrutinib is well tolerated, similar to Compound 1 monotherapy. Risk of TLS appears limited and manageable, including laboratory TLS has been seen in only 1 patient with high TLS-risk CLL receiving monotherapy.

In addition, transient neutropenia was the most frequent grade ≥3 AE, and substantial decreases in ALC have been seen during ramp-up for patients with CLL, with promising early response rates among patients with R/R CLL.

In one embodiment of each of the above aspects, the Bcl-2 is orally administrated once daily (QD).

100321 In one embodiment of each of the above aspects, the B-cell malignancy has Bcl-2 expression.

In one embodiment of each of the above aspects, the B-cell malignancy has Bcl-2 Gly101Val mutation expression.

### BRIEF DESCRIPTION OF THE DRAWINGS

FIGS. 1A and 1B show the efficacy of Bcl-2 inhibitors in RS4:11 acute lymphoblastic leukemia (ALL) subcutaneous xenograft model. ####p<0.0001 versus vehicle by one-way ANOVA (Dunnett's multiple comparisons test)

* p<0.05, **** p<0.0001 versus venetoclax by one-way ANOVA (Tukey's multiple comparisons test). Abbreviation: ANOVA, analysis of variance; SEM, standard error of the mean; QD, once daily; BID, twice daily; p.o., oral gavage.

FIGS. 2A and 2B show the efficacy of Bcl-2 inhibitors in MAVER-1 mantle cell lymphoma (MCL) subcutaneous xenograft model. ## p<0.01, #### p<0.0001 versus vehicle by one-way ANOVA (Dunnett's multiple comparisons test). **** p<0.0001 versus venetoclax by one-way ANOVA (Tukey's multiple comparisons test). Abbreviation: ANOVA, analysis of variance; SEM, standard error of the mean; QD, once daily; BID, twice daily; p.o., oral gavage.

FIGS. 3A and 3B show the efficacy of Bcl-2 inhibitors in Toledo diffuse large B cell lymphoma (DLBCL) subcutaneous xenograft model. ### p<0.001, #### p<0.0001 versus vehicle by one-way ANOVA (Dunnett's multiple comparisons test).

** p<0.01, **** p<0.0001 versus venetoclax by one-way ANOVA (Tukey's multiple comparisons test). Abbreviation: ANOVA, analysis of variance; SEM, standard error of the mean; QD, once daily; BID, twice daily; p.o., oral gavage.

FIGS. 4A and 4B show the efficacy of Bcl-2 inhibitors in RS4;11 Bcl-2G101V KI acute lymphoblastic leukemia (ALL) subcutaneous xenograft model. ##p<0.01, ####p<0.0001 versus vehicle by one-way ANOVA; **** p<0.0001 versus venetoclax by one-way ANOVA.

FIGS. 5A-5D show the effect of Compound 1 (Bcl-2 inhibitor) and Compound B (BTK inhibitor) on tumor growth in human JeKo-1 MCL xenograft model. * p<0.05, *** p<0.001, ****p<0.0001 versus combo treatment group by One-way ANOVA test.

FIG. 6A shows the treatment-emergent AEs regardless of causality occurring in at least 2 patients receiving (A) monotherapy (N=25) or (B) combination Therapy (N=11). In FIG. 6A: ^{a}Neutropenia combines “neutrophil count decreased” and“neutropenia”; ^{b}Thrombocytopenia combines “platelet count decreased” and “thrombocytopenia”; and ALT=alanine transaminase.

FIG. 6B shows the duration of treatment and best response. In FIG. 6B: ^{a}Duration of treatment includes 8-12 weeks of zanubrutinib monotherapy prior to initiation of Compound 1+zanubrutinib combination; nPR=nodular partial response; PD=progressive disease; PR=partial response; PR-L=PR with lymphocytosis; and SD=stable disease.

FIG. 6C shows the change in SPD among patients with NHL^{a}. In FIG. 6C. ^{a}Includes all patients from Cohort 1A that had a post baseline CT scan as of data cutoff (n=11); CT=computed tomography; DLBCL=diffuse large B-cell lymphoma; FL=follicular lymphoma; MZL=marginal zone lymphoma; and SPD=sum of product of perpendicular diameter.

FIG. 6D shows reduction in ALC over ramp-up in patients with CLL^{a}. In FIG. 6D, ^{a}Figures represent reduction in ALC above the ULN (4×109/L) compared to pre-Compound 1 baseline before next dose escalation (or after 1 week at target dose) per dose. Patients receive each Compound 1 dose level for 1 week before escalating to the next dose. Patients on combination therapy were also receiving zanubrutinib during Compound 1 ramp-up, beginning 8-12 weeks before the first Compound 1 dose (note: 1 patient with normal baseline ALC was excluded from the monotherapy figure).

### DEFINITIONS

Unless specifically defined elsewhere in this document, all other technical and scientific terms used herein have the meaning commonly understood by one of ordinary skill in the art.

As used herein, including the appended claims, the singular forms of words such as “a,” “an,” and “the,” include their corresponding plural references unless the context clearly dictates otherwise.

The term “or” is used to mean, and is used interchangeably with, the term “and/or” unless the context clearly dictates otherwise.

The term “anti-cancer agent” as used herein refers to any agent that can be used to treat a cell proliferative disorder such as cancer, including but not limited to, cytotoxic agents, chemotherapeutic agents, radiotherapy and radiotherapeutic agents, targeted anti-cancer agents, and immunotherapeutic agents.

The terms “administration,” “administering,” “treating,” and “treatment” herein, when applied to an animal, human, experimental subject, cell, tissue, organ, or biological fluid, means contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, rabbit) and most preferably a human. Treating any disease or disorder refer in one aspect, to ameliorating the disease or disorder (i.e., slowing or arresting or reducing the development of the disease or at least one of the clinical symptoms thereof). In another aspect, “treat,” “treating.” or “treatment” refers to alleviating or ameliorating at least one physical parameter including those which may not be discernible by the patient. In yet another aspect, “treat,” “treating,” or “treatment” refers to modulating the disease or disorder, either physically, (e.g., stabilization of a discernible symptom), physiologically, (e.g., stabilization of a physical parameter), or both. In yet another aspect. “treat,” “treating,” or “treatment” refers to preventing or delaying the onset or development or progression of the disease or disorder.

The term “subject” in the context of the present disclosure is a mammal, e.g., a primate, preferably a higher primate, e.g., a human (e.g., a patient having, or at risk of having, a disorder described herein). In some embodiments, the subject is a human or a patient.

The terms “cancer” or “tumor” herein has the broadest meaning as understood in the art and refers to the physiological condition in mammals that is typically characterized by unregulated cell growth. In the context of the present disclosure, the cancer is not limited to a certain type or location.

The term “therapeutically effective amount” as herein used, refers to the amount of a Bcl-2 inhibitor that, when administered to a subject for treating a disease, or at least one of the clinical symptoms of a disease or disorder, is sufficient to effect such treatment for the disease, disorder, or symptom. The “therapeutically effective amount” can vary with the agent, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In the case of combination therapy, the “therapeutically effective amount” refers to the total amount of the combination objects for the effective treatment of a disease, a disorder or a condition.

The term “ramp-up scheme” or “ramp-up schedule” as herein used, refer to a dosing scheme or schedule wherein the active ingredient of interest is administrated at a dose increased at a regular basis such as daily or weekly for a designated period such as server days or several weeks, and then is administrated to the recommended dose (daily or weekly).

### DETAILED DESCRIPTION OF THE DISCLOSURE

The present disclosure provides a method of treating B-cell malignancy in a subject with Bcl-2 inhibitor, in particularly 2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide (Compound 1) or a pharmaceutically acceptable salt thereof.

The present disclosure also provides a method of treating B-cell malignancies in a subject, comprising administering to the subject in need thereof a therapeutically effective amount of a Bcl-2 inhibitor, or a stereoisomer thereof, or a pharmaceutically acceptable salt thereof, in combination with a therapeutically effective amount of (S)-7-(1-acryloylpiperidin-4-yl)-2-(4-phenoxyphenyl)-4,5,6,7-tetra-hydropyrazolo[1,5-a]pyrimidine-3-carboxamide (Compound B) or a pharmaceutically acceptable salt thereof.

### Bcl-2 Inhibitor

The Bcl-2 inhibitor in the present disclosure is a compound represented by the following Formulas (III-B), (III-C), (III-D) or (III-E),

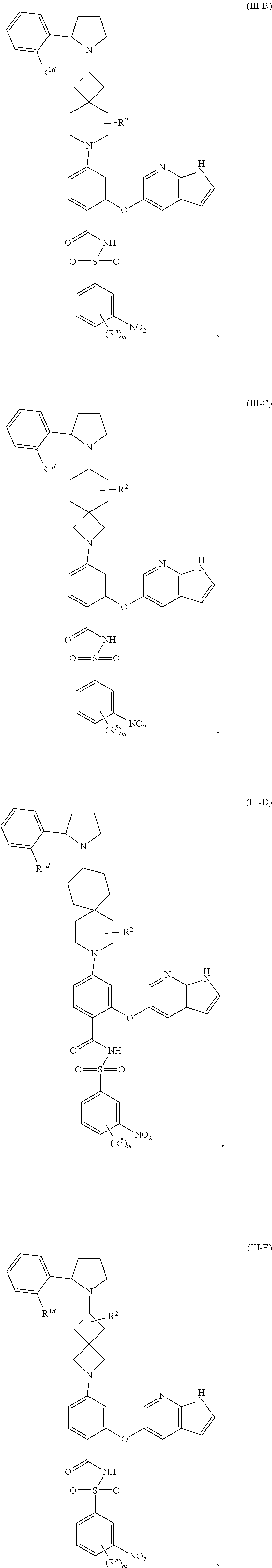

or a pharmaceutically acceptable salt thereof, or a stereoisomer thereof.
wherein,
R², at each occurrence, is independently selected from the group consisting of hydrogen, halogen, and —C₁₋₈alkyl optionally substituted with halogen;
R^{1d}, at each occurrence, is independently halogen, —C₁₋₈alkyl, —C₂₋₈alkenyl, —C₂₋₈alkynyl, cycloalkyl, heterocyclyl, aryl, heteroaryl, oxo, —CN, —NO₂, —OR^{Ba}, —SO₂R^{Ba}, —COR^{Ba}, —CO₂R^{Ba}, —CONR^{Ba}R^{Bb}, —C(═NR^{Ba})NR^{Bb}R^{Bc}, —NR^{Ba}R^{Bb}, —NR^{Ba}COR^{Bb}, —NR^{Ba}CONR^{Bb}R^{Bc}, —NR^{Ba}CO₂R^{Bb}, —NR^{Ba}SONR^{Bb}R^{Bc}, —NR^{Ba}SO₂NR^{Bb}R^{Bc}, or —NR^{Ba}SO₂R^{Bb}; wherein said —C₁₋₈alkyl, —C₂₋₈alkenyl, —C₂₋₈alkynyl, cycloalkyl, heterocyclyl, aryl or heteroaryl are each independently optionally substituted with 1 to 4 substituents R^{Bd};
R^{Ba}, R^{Bb}, and R^{Bc}, are each independently hydrogen, —C₁₋₈alkyl, —C₂₋₈alkenyl, —C₂₋₈alkynyl, cycloalkyl, heterocyclyl, aryl, or heteroaryl, each of said —C₁₋₈alkyl, —C₂₋₈alkenyl, —C₂₋₈alkynyl, cycloalkyl, heterocyclyl, aryl, or heteroaryl is optionally substituted with halogen, hydroxy, —NH₂ or —N(C₁₋₆alkyl)₂, —C₁₋₈alkyoxy, cycloalkyl, heterocyclyl, aryl, or heteroaryl;
R^{Bd}, at each occurrence, is independently hydrogen, halogen, oxo, —CN, —NO₂, —C₁₋₈alkyl, —C₂₋₈alkenyl, —C₂₋₈alkynyl, cycloalkyl, heterocyclyl, aryl, or heteroaryl, each of said —C₁₋₈alkyl, —C₂₋₈alkenyl, —C₂₋₈alkynyl, cycloalkyl, heterocyclyl, aryl, or heteroaryl is optionally substituted with halogen, hydroxy, —C₁₋₈alkyoxy, cycloalkyl, heterocyclyl, aryl, or heteroaryl;
m is an integer of 14;
R⁵ is -L⁵-CyC,
   Wherein L⁵ is a direct bond, —(CR^{a}R^{b})ₜ—, —(CR^{a}R^{b})ₜ₋₁—(CR^{c}═CR^{d})—(CR^{a}R^{b})ᵥ₋₁—, —(CR^{a}R^{b})ₜ₋₁—(C≡C)—(CR^{a}R^{b})ᵥ₋₁—, —O—, —S—, —S(O)—, —SO₂—, —C(O)—, C(O)O—, —OC(O)—, —NR^{a}—, —C(O)NR^{a}—, —NR^{a}C(O)—, —NR^{a}C(O)O—, —NR^{a}C(O)NR^{b}—, —SO₂NR^{a}—, —NR^{a}S(O)₂NR^{b}—, —NR^{a}S(O)NR^{b}—, —C(O)NR^{a}SO₂, —C(O)NR^{a}SO—, or —C(═NR^{a})NR^{b}—, wherein t and v, at each occurrence, are independently a number of 1 to 7, and one or two CR^{a}R^{b} moieties in —(CR^{a}R^{b})ᵣ, —(CR^{a}R^{b})ₜ₋₁—(CR^{c}═CR^{d})—(CR^{a}R^{b})ᵥ₋₁—, —(CR^{a}R^{b})ₜ₋₁—(C≡C)—(CR^{a}R^{b})ᵥ₋₁— are un-replaced or replaced with one or more moieties selected from O, S, SO, SO₂, C(O) or NR^{a};
   CyC is cycloalkyl, heterocyclyl, aryl, or heteroaryl, each of which is optionally substituted with one or two substituents R^{5a};
   R^{5a}, at each occurrence, is independently selected from hydrogen, halogen, cyano, oxo, —NO₂, —OR^{5b}, —SR^{5b}, —NR^{5b}R^{5c}, —COR^{5b}, —SO₂R^{5b}, —C(═O)OR^{5b}, —C(═O)NR^{5b}R^{5c}, —C(═NR^{5b})NR^{5c}R^{5d}, —N(R^{5b})C(═O)R^{5c}, —N(R^{5b})C(═O)OR^{5c}, —N(R^{5b})C(O)NR^{5c}R^{5d}, —N(R^{5b})S(O)NR^{5c}R^{5d}, —N(R^{5b})S(O)₂NR^{5c}R^{5d}, —NR^{5b}SO₂R^{5c}, —C₁₋₈alkyl, —C₂₋₈alkenyl, —C₂₋₈alkynyl, -cycloalkyl, heterocyclyl, aryl, or heteroaryl, each of said —C₁₋₈alkyl, —C₂₋₈alkenyl, —C₂₋₈alkynyl, -cycloalkyl, heterocyclyl, aryl, or heteroaryl is optionally substituted with one or two substituents R^{5e};
      wherein R^{5b}, R^{5c}, and R^{5d} are each independently hydrogen, —C₁₋₈alkyl, —C₂₋₈alkenyl, —C₂₋₈alkynyl, cycloalkyl, heterocyclyl, aryl, or heteroaryl, each of said —C₁₋₈alkyl, —C₂₋₈alkenyl, C₂₋₈alkynyl, -cycloalkyl, heterocyclyl, aryl, or heteroaryl is optionally substituted with one or two substituents R^{5e};
      R^{5e}, at each occurrence, is independently selected from hydrogen, halogen, cyano, oxo, —NO₂, —OR^{5f}, —SR^{5f}, —NR^{5f}R^{5g}, —COR^{5f}, —SO₂R^{5f}, —C(═O)OR^{5f}, —C(═O)NR^{5f}R^{5g}, —C(═NR^{5f})NR^{5g}R^{5h}, —N(R^{5f})C(═O)R^{5g}, —N(R^{5f})C(═O)OR^{5g}, —N(R^{5f})C(O)NR^{5g}R^{5h}, —N(R^{5f})S(O)NR^{5g}R^{5h}, —N(R^{5f})S(O)₂NR^{5g}R^{5h}, —NR^{5f}SO₂R^{5g}, —C₁₋₈alkyl, —C₂₋₈alkenyl, heterocyclyl, aryl, or heteroaryl;
         R^{5f}, R^{5g}, and R^{5h} are each independently hydrogen, —C₁₋₈alkyl, —C₂₋₈alkenyl, —C₂₋₈alkynyl, cycloalkyl, heterocyclyl, aryl, or heteroaryl;
or, two adjacent R⁵ on the phenyl ring together with the phenyl ring form a benzo ring, said ring is optionally substituted with halogen, oxo, cyano, —NO₂, —OR⁵ⁱ, —SR⁵ⁱ, —NR⁵ⁱR^{5j}, —COR⁵ⁱ, —SO₂R⁵ⁱ, —C(═O)OR⁵ⁱ, —C(═O)NR⁵ⁱR^{5j}, —C(═NR⁵ⁱ)NR^{5j}R^{5k}, —N(R⁵ⁱ)C(═O)R^{5j}, —N(R⁵ⁱ)C(═O)OR^{5j}, —N(R⁵ⁱ)C(O)NR^{5j}R^{5k}, —N(R⁵ⁱ)S(O)NR^{5j}R^{5k}, —N(R⁵ⁱ)S(O)₂NR^{5j}R^{5k}, —NR⁵ⁱSO₂R^{5k}, —C₁₋₈alkyl, —C₂₋₈alkenyl, —C₂₋₈alkynyl, -cycloalkyl, heterocyclyl, aryl, or heteroaryl;
   R⁵ⁱ, R^{5j}, and R^{5k} are independently hydrogen, —C₁₋₈alkyl, —C₂₋₈alkenyl, —C₂₋₈alkynyl, cycloalkyl, heterocyclyl, aryl, or heteroaryl, each of said —C₁₋₈alkyl, —C₂₋₈alkenyl, —C₂₋₈alkynyl, cycloalkyl, heterocyclyl, aryl, or heteroaryl is optionally substituted with halogen, hydroxy or —C₁₋₈alkyoxy;
R^{a}, R^{b}, R^{c}, and R^{d} at each occurrence, are independently hydrogen, —C₁₋₈alkyl, —C₂₋₈alkenyl, —C₂₋₈alkynyl, cycloalkyl, heterocyclyl, aryl, or heteroaryl, said —C₁₋₈alkyl, —C₂₋₈alkenyl, —C₂₋₈alkynyl, cycloalkyl, heterocyclyl, aryl, or heteroaryl are each independently substituted with —CN, halogen, —NO₂, —NR^{e}R^{f}, oxo, —OR^{e}, or —SR^{e}; and
wherein R^{e} and R^{f} are each independently hydrogen, C₁₋₈alkyl, C₁₋₈alkoxy-C₁₋₈alkyl-, C₂₋₈alkenyl, C₂₋₈alkynyl, cycloalkyl, aryl, heterocyclyl, or heteroaryl.

In some embodiments, R² is hydrogen.

In some embodiments, R^{1d}, when substituted on the phenyl group at position 2 of ring B (including the aziridin-1-yl, azetidin-1-yl, pyrrolidin-1-yl, pyrrolidin-2-yl, piperidin-1-yl, azepan-1-yl, or azocan-1-yl, preferably the pyrrolidin-1-yl group), is independently halogen, —C₁₋₈alkyl, —C₂₋₈alkenyl, —C₂₋₈alkynyl, cycloalkyl, heterocyclyl, aryl, heteroaryl, —CN, —OR^{Ba}, —SO₂R^{Ba}, —CONR^{Ba}R^{Bb}, —NO₂, —NR^{Ba}R^{Bb}, —NR^{Ba}COR^{Bb}, or —NR^{Ba}SO₂R^{Bb}; wherein said —C₁₋₈alkyl, —C₂₋₈alkenyl, —C₂₋₈alkynyl, cycloalkyl, heterocyclyl, aryl or heteroaryl are each independently optionally substituted with 1 to 4 substituents R^{Bd} as defined as with Formulas (III-B), (III-C), (III-D) or (III-E), preferably 1 or 2 substituents R^{Bd} as defined as with Formulas (III-B), (III-C), (III-D) or (III-E). In another aspect, one R^{1d} is at position 2 of the phenyl ring at position 2 of ring B.

In some embodiments, R^{1d} is methyl, ethyl, isopropyl, propyl or methoxymethyl, or two methyl at the position of the phenyl ring; or propenyl; or cyclopropyl, cyclobutyl, cyclopentyl, or cyclohexyl; or ethoxy or isopropoxy; or amino or dimethylamino.

In some embodiments, the 2-(2-substituted phenyl)pyrrolidin-1-yl moiety in Formulas (III-B), (III-C), (III-D) or (III-E), is selected from the group consisting of:

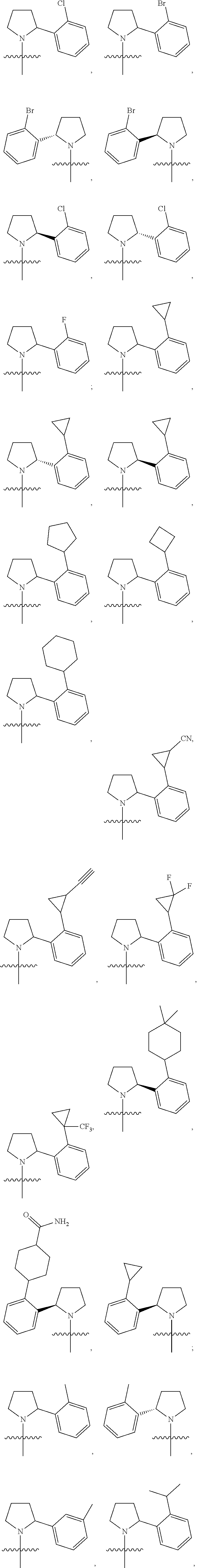

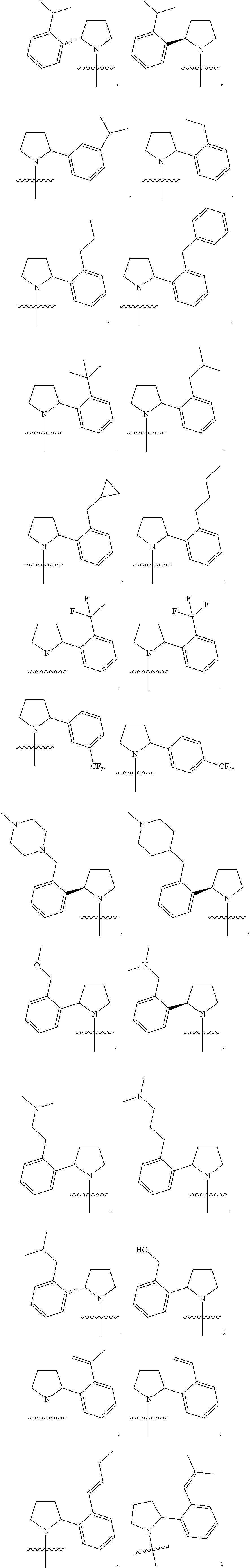

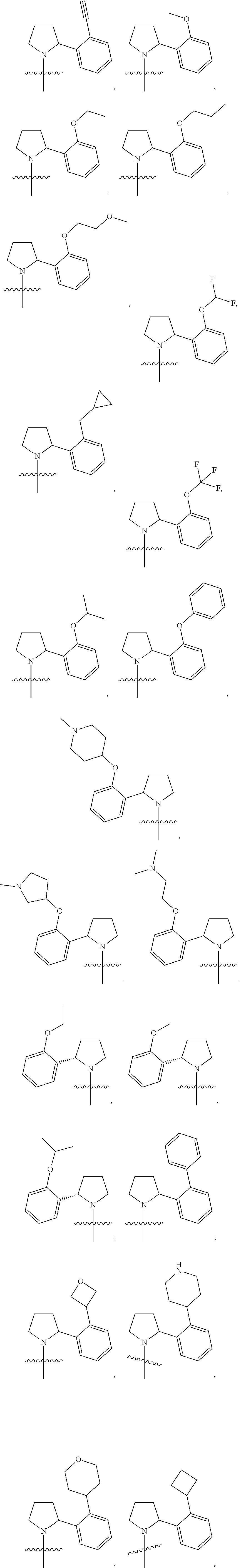

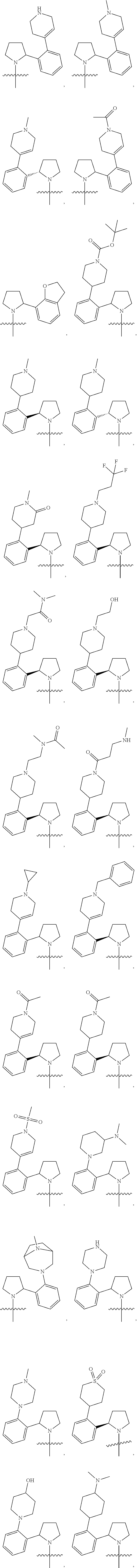

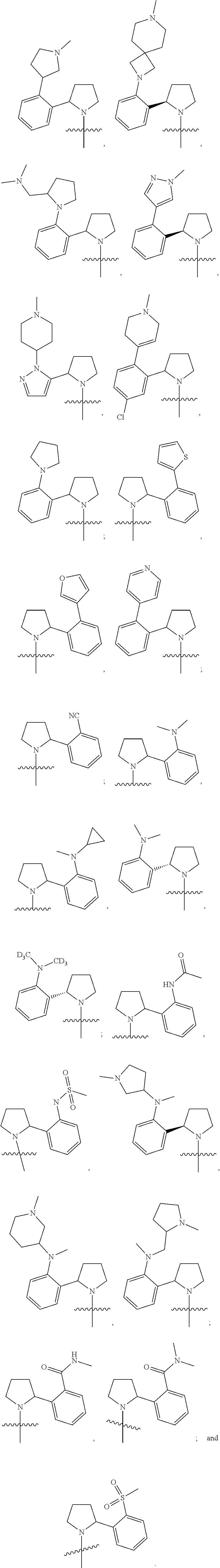

In some embodiments, m is 1; and L⁵ is a direct bond, —(CR^{a}R^{b})ₜ— or —NR^{a}—, wherein t is a number of 1 to 7, and one or two CR^{a}R^{b} moieties in (CR^{a}R^{b})ₜ— are un-replaced or replaced with one or more moieties selected from O or NR^{a}, wherein R^{a} and R^{b} are defined with Formulas (III-B), (III-C), (III-D) or (III-E).

In some embodiments, L⁵ is a direct bond, —(CR^{a}R^{b})₁₋₄—, —O—(CR^{a}R^{b}))₁₋₃—, —NH—(CR^{a}R^{b})₁₋₃, or —NH—, wherein R^{a} and R^{b} are defined as with Formulas (III-B), (III-C), (III-D) or (III-E), so that the -L⁵-CyC moiety is CyC, —(CR^{a}R^{b})₁₋₄-CyC, —O—(CR^{a}R^{b})₁₋₃-CyC, —NH—(CR^{a}R^{b})₁₋₃-CyC, or —NH-CyC, respectively. More preferably, L⁵ is a direct bond, —(CH₂)₁₋₄—, —O—(CH₂)₁₋₃—, —NH—(CR^{a}R^{b})—(CH₂)₂—, or —NH—, wherein R^{a} is hydrogen and R^{b} is C₁₋₈alkyl optionally substituted with phenyl-S— so that the -L⁵-CyC moiety is CyC, —(CH₂)₁₋₄-CyC, —O—(CH₂)₁₋₃-CyC, —NH—(CR^{a}R^{b})—(CH₂)₂-CyC, or —NH-CyC, respectively. More preferably, L⁵ is a direct bond, —CH₂—, —O—CH₂—, —NH—CH₂—, or —NH— so that the -L⁵-CyC moiety is CyC, —CH₂-CyC, —O—CH₂-CyC, —NH—CH₂-CyC, or —NH-CyC, respectively.

In some embodiments, CyC is cycloalkyl, or heterocyclyl, each of which is optionally substituted with one or two substituents R^{5a};
R^{5a} is independently selected from hydrogen, halogen, cyano, oxo, —OR^{5b}, —NR^{5b}R^{5c}, —COR^{5b}, —SO₂R^{5b}, —C₁₋₈alkyl, —C₂₋₈alkynyl, -cycloalkyl, or heterocyclyl, each of said —C₁₋₈alkyl, and heterocyclyl is optionally substituted with one or two substituents R^{5e} which is selected from hydrogen, halogen, cyan, —OR^{5f}, —C₁₋₈alkyl, -cycloalkyl, or heterocyclyl:
   wherein R^{5b}, and R^{5c} are each independently hydrogen, —C₁₋₈alkyl or heterocyclyl, said —C₁₋₈alkyl is optionally substituted with one or two substituents R^{5e} which is hydrogen, —NR^{5f}R^{5g}, or -cycloalkyl;
      R^{5f} and R^{5g} are each independently hydrogen or —C₁₋₈alkyl;
or, two adjacent R⁵ on the phenyl ring together with the phenyl ring form a benzo ring, said ring is optionally substituted with heteroaryl.

In some embodiments, CyC is cycloalkyl selected from monocyclic C₃₋₈cycloalkyl or bridged cycloalkyl

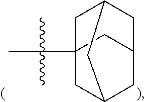 [SMILES: *C12CC3CC(CC(C3)C1)C2]

each of which is optionally substituted with one or two substituents R^{5a}, preferably, CyC is cyclopentyl or cyclohexyl, each of which is optionally substituted with one or two substituents R^{5a}.

In some embodiments, CyC is heterocyclyl selected from:
a) monocyclic 4 to 9-membered heterocyclyl groups containing one nitrogen or oxygen or sulfur heteroatom as ring member;
b) monocyclic 4 to 9-membered heterocyclyl groups containing two heteroatoms selected from oxygen, sulfur or nitrogen as ring members; or
c) 5 to 20-membered Spiro heterocyclyl comprising one or two heteroatoms selected from nitrogen, sulfur or oxygen as ring members,
each of which is optionally substituted with one or two R^{5a}.

In some embodiments, CyC is monocyclic 4 to 6-membered heterocyclyl groups containing one nitrogen or oxygen or sulfur heteroatom as the ring member. More preferably, Cyc is selected from oxetanyl, tetrahydrofuranyl, tetrahydropyranyl, azetidinyl, pyrrolidinyl, or piperdinyl. Even more preferably, CyC is selected from oxetan-2-yl, Oxetan-3-yl, tetrahydrofuran-4-yl, tetrahydrofuran-2-yl, tetrahydrofuran-3-yl, tetrahydropyran-2-yl, tetrahydropyran-3-yl, tetrahydropyran-4-yl, azetidin-3-yl, azetidin-2-yl, pyrrolidin-2-yl, pyrrolidin-3-yl, piperdin-4-yl, piperdin-2-yl, or piperdin-3-yl.

In some embodiments, CyC is a monocyclic 6-membered heterocyclyl group containing two heteroatoms selected from oxygen or nitrogen as ring members. More preferably, CyC is dioxanyl, morpholino, morpholinyl, or piperzinyl. Even more preferably 1,3-dioxan-2-yl, 1,3-dioxan-4-yl, 1,4-dioxan-2-yl, morpholin-1-yl, morpholin-2-yl, or morpholin-3-yl.

In some embodiments, R^{5a} is independently selected from hydrogen, halogen, cyano, oxo, —OR^{5b}, —NR^{5b}R^{5c}, —COR^{5b}, —SO₂R^{5b}, —C₁₋₈alkyl, —C₂₋₈alkynyl, monocyclic C₃₋₈cycloalkyl, or monocyclic 4 to 9-membered heterocyclyl group containing one or two heteroatoms selected from nitrogen or oxygen or sulfur heteroatom as ring members, each of said —C₁₋₈alkyl and monocyclic 4 to 9-membered heterocyclyl group is optionally substituted with one or two substituents R^{5e}; preferably, cycloalkyl as R^{5a} is C₃₋₆cycloalkyl; more preferably cyclopropyl; preferably, heterocyclyl as R^{5a} is 4 to 6-membered heterocyclyl groups containing one or two heteroatoms selected from nitrogen or oxygen or sulfur heteroatom as ring members; more preferably, heterocyclyl as R^{5a} is oxetanyl, tetrahydrofuranyl, tetrahydropyranyl, piperzinyl, or morpholinyl; even more preferably, heterocyclyl as R^{5a} is oxetan-3-yl, tetrahydrofuran-3-yl, tetrahydro-2H-pyran-4-yl, or morphin-4-yl.

In some embodiments, heterocyclyl as R^{5e} is a monocyclic 4 to 9-membered heterocyclyl group containing one or two heteroatoms selected from nitrogen or oxygen or sulfur heteroatom as ring members.

In some embodiments, heterocyclyl as R^{5e} is tetrahydro-pyran-4-yl.

In some embodiments, R^{5a} is —NR^{5b}R^{5c}, wherein R^{5b} is hydrogen, and R^{5c} is heterocyclyl.

In some embodiments, R^{5a} is —NR^{5b}R^{5c}, wherein R^{5b} is hydrogen, and R^{5c} is tetrahydro-pyran-4-yl.

In some embodiments, R^{5a} is —NR^{5b}R^{5c}, wherein R^{5b} and R^{5c} are each independently hydrogen or —C₁₋₆alkyl substituted with cycloalkyl, preferably —C₁₋₆alkyl substituted with monocyclic C₃₋₈cycloalkyl.

In some embodiments, R^{5a} is —OR^{5b} or —SO₂R^{5b}, wherein R^{5b} is hydrogen or C₁₋₈alkyl, preferably methyl.

In some embodiments, R^{5a} is —COR^{5b}, wherein R^{5b} is hydrogen or C₁₋₈alkyl optionally substituted with —NR^{5f}R^{5g}, wherein R^{5f} and R^{5g} are each independently hydrogen or C₁₋₈alkyl, preferably methyl.

In some embodiments, two adjacent R⁵ on the phenyl ring together with the phenyl ring form indazolyl which is substituted with tetrahydropyranyl.

In some embodiments, m is 1, and R⁵ is -L⁵-CyC selected from the group consisting of:

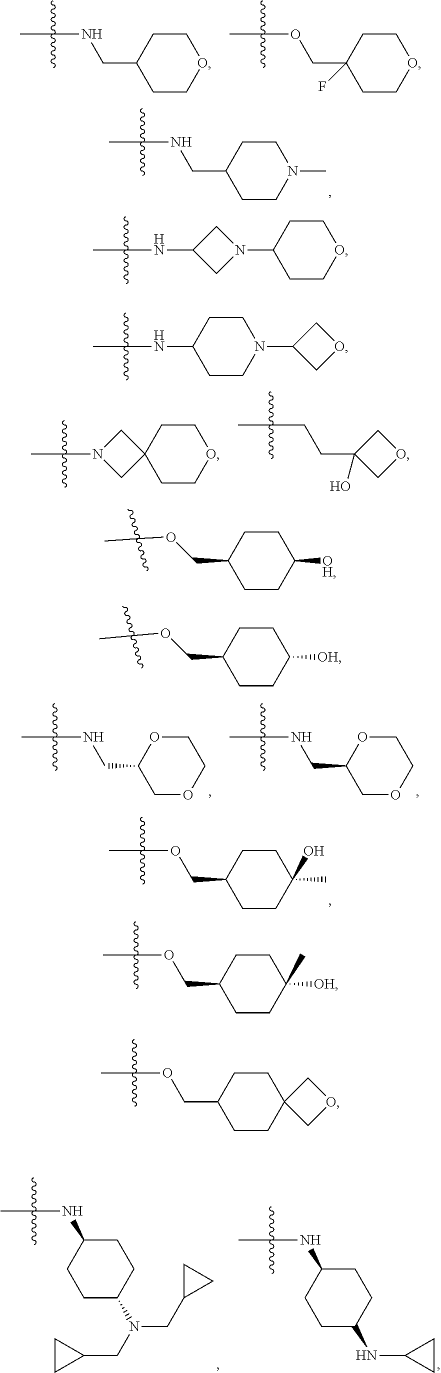

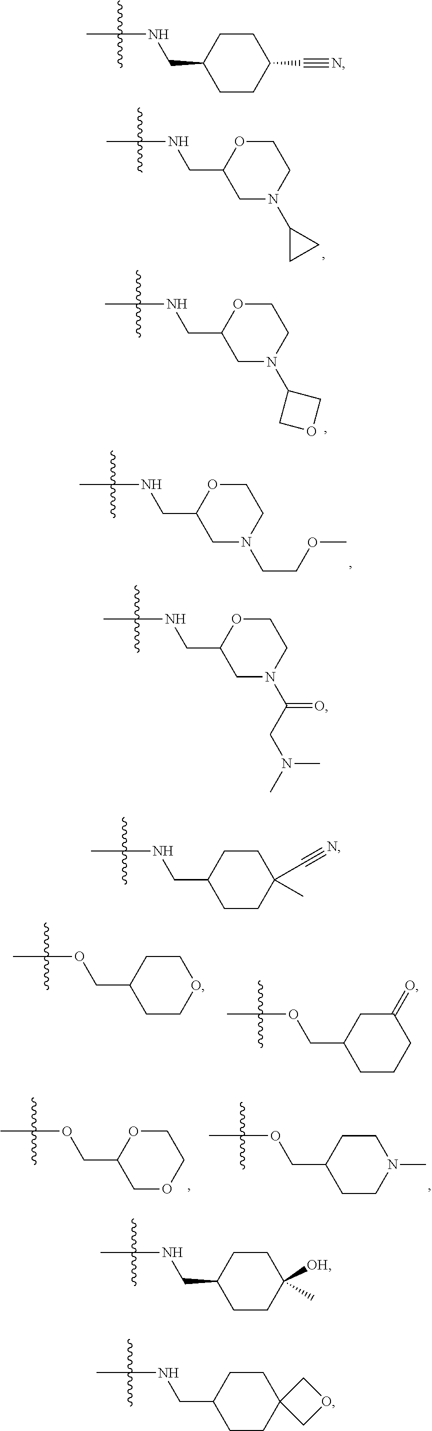

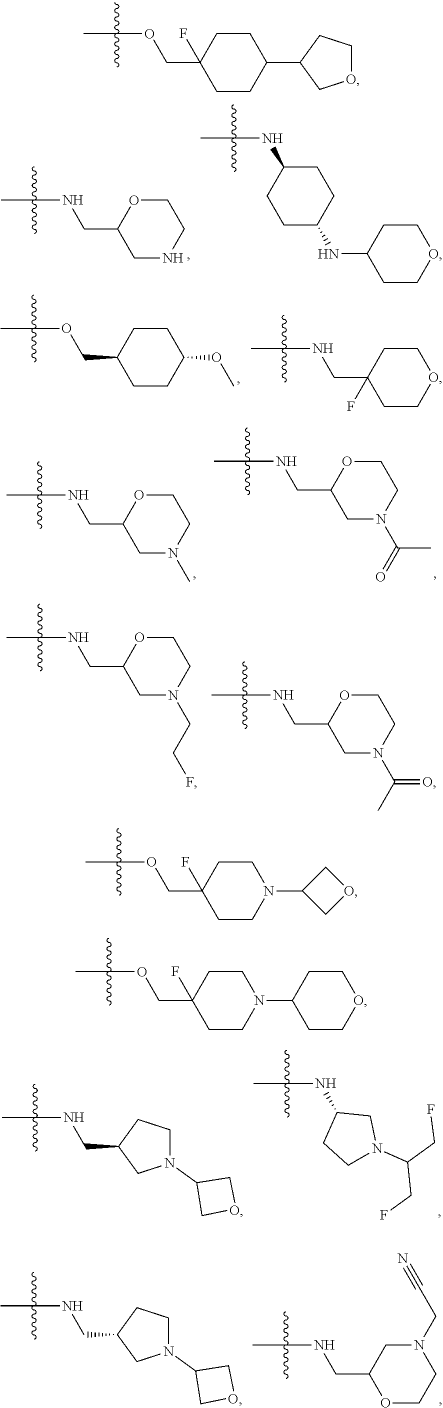

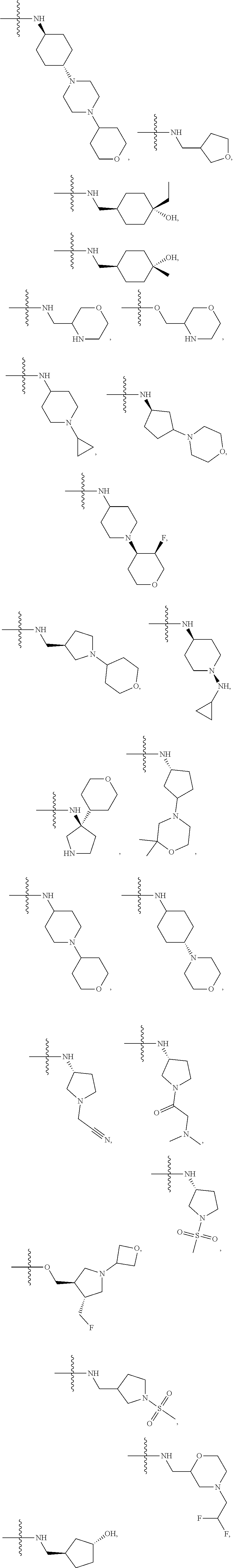

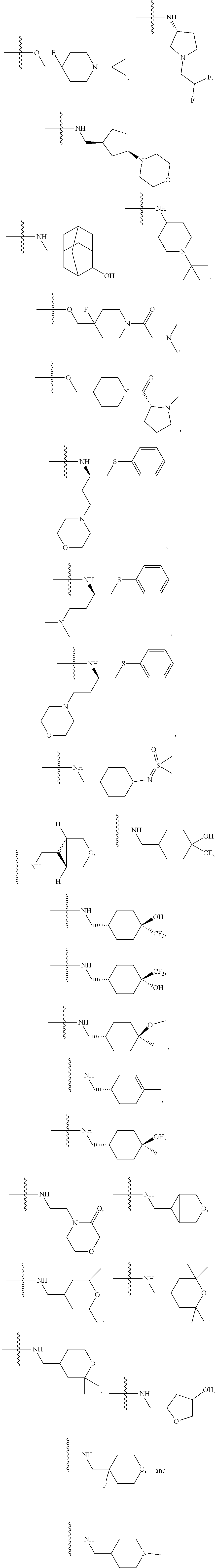

In some embodiments, m is 1 and R⁵ is

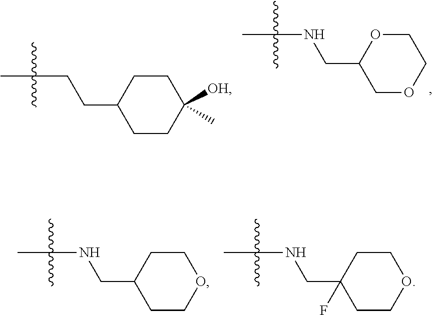

In some embodiments, the Bcl-2 inhibitor in present disclosure is selected form the group consisting of:
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((4-(((4-fluorotetrahydro-2H-pyran-4-yl)methyl)amino)-3-nitrophenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-1-(2-(2-(2-ethylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-ethylphenyl)pyrrolidin-1-yl)-7-azaspiro[3.5]nonan-7-yl)-N-((4-(((4-fluorotetrahydro-2H-pyran-4-yl)methyl)amino)-3-nitrophenyl)sulfonyl)benzamide;
2-((1H-pyrrolo[2,3-b]pyridin-5-yl)oxy)-4-(2-((S)-2-(2-ethylphenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
(R)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((4-(((4-fluorotetrahydro-2H-pyran-4-yl)methyl)amino)-3-nitrophenyl)sulfonyl)benzamide;
2-((1H-pyrrolo[2,3-b]pyridin-5-yl)oxy)-4-(2-((R)-2-(2-cyclopropylphenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
(S)-2-((1H-pyrrolo[2,3-b]pyridin-5-yl)oxy)-4-(7-(2-(2-cyclopropylphenyl)pyrrolidin-1-yl)-2-azaspiro[3.5]nonan-2-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(7-(2-(2-cyclopropylphenyl)pyrrolidin-1-yl)-2-azaspiro[3.5]nonan-2-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(9-(2-(2-cyclopropylphenyl)pyrrolidin-1-yl)-3-azaspiro[5.5]undecan-3-yl)-N-((3-nitro-4-(((tetrahydro-2H-pyran-1-yl)methyl)amino)phenyl)sulfonyl)benzamide;
(R)-2-((1H-pyrrolo[2,3-b]pyridin-5-yl)oxy)-4-(9-(2-(2-cyclopropylphenyl)pyrrolidin-1-yl)-3-azaspiro[5.5]undecan-3-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(6-(2-(2-cyclopropylphenyl)pyrrolidin-1-yl)-2-azaspiro[3.3]heptan-2-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(6-(2-(2-cyclopropylphenyl)pyrrolidin-1-yl)-2-azaspiro[3.3]heptan-2-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(3-chloro-2-(dimethylamino)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)benzamide;
N-((4-((((R)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)benzamide;
(S)2-((1H-pyrrolo[2,3-b]pyridin-5-yl)oxy)-4-(2-((S)-2-(2-cyclopropylphenyl)pyrrolidin-1-yl)-8-azaspiro[4.5]decan-8-yl)-N-((3-nitro-4-(((tetrahydro-2H-pyran-4-yl)methyl)amino)phenyl)sulfonyl)benzamide;
(R) 2-((1H-pyrrolo[2,3-b]pyridin-5-yl)oxy)-4-(2-((S)-2-(2-cyclopropylphenyl)pyrrolidin-1-yl)-8-azaspiro[4.5]decan-8-yl)-N-((3-nitro-4-(((tetrahydro-2H-pyran-4-yl)methyl)amino)phenyl)sulfonyl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-ethylphenyl)pyrrolidin-1-yl)-7-azaspiro[3.5]nonan-7-yl)benzamide;
N-((4-((((R)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-ethylphenyl)pyrrolidin-1-yl)-7-azaspiro[3.5]nonan-7-yl)benzamide;
(S)-2-((1H-pyrrolo[2,3-b]pyridin-5-yl)oxy)-4-(8-(2-(2-cyclopropylphenyl)pyrrolidin-1-yl)-2-azaspiro[4.5]decan-2-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-isopropylphenyl)pyrrolidin-1-yl)-7-azaspiro[3.5]nonan-7-yl)benzamide;
(S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-N-((4-(((4-fluorotetrahydro-2H-pyran-4-yl)methyl)amino)-3-nitrophenyl)sulfonyl)-4-(2-(2-(2-isopropylphenyl)pyrrolidin-1-yl)-7-azaspiro[3.5]nonan-7-yl)benzamide;
N-((4-((((R)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-N-((4-((((1s,4s)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((R)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-N-((4-((((1s,4s)-4-hydroxy-4-methylcyclohexyl)methyl)amino)-3-nitrophenyl)sulfonyl)-4-(2-((R)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-4-(2-(2-(2-cyclobutylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-isobutylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(3-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-N-((3-nitro-4-(((tetrahydro-2H-pyran-4-yl)methyl)amino)phenyl)sulfonyl)-4-(2-(2-(o-tolyl)pyrrolidin-1-yl)-7-azaspiro[3.5]nonan-7-yl)benzamide;
(S)-2-((1H-pyrrolo[2,3-b]pyridin-5-yl)oxy)-4-(2-(2-(2-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-bromophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(2-(2-(2-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(3-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(2-(2-(3-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(4-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-ethoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-(dimethylamino)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-((S)-2-(2-(dimethylamino)phenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-(2-((S)-2-(2-(bis(methyl-d3)amino)phenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-N-((3-nitro-4-(((tetrahydro-2H-pyran-4-yl)methyl)amino)phenyl)sulfonyl)-4-(2-(2-(2-(pyrrolidin-1-yl)phenyl)pyrrolidin-1-yl)-7-azaspiro[3.5]nonan-7-yl)benzamide;
(S)-2-((1H-pyrrolo[2,3-b]pyridin-5-yl)oxy)-4-(2-(2-(2-(1-methyl-1,2,3,6-tetrahydropyridin-4-yl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-(1-methylpiperidin-4-yl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-methoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-(methoxymethyl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-(hydroxymethyl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(2-(2-(3-chloro-2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(3-chloro-2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(5-chloro-2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(2-(2-(3-chloro-2-ethylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S or R)-2-((1H-pyrrolo[2,3-b]pyridin-5-yl)oxy)-4-(2-(2-(3-chloro-2-ethylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2,4-dicyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-1-(2-(2-(2,5-dicyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(3-(2-chlorophenyl)thiophen-2-yl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4-methylpyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(4-cyclopropyl-2-(2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4-phenyl-2,5-dihydro-1H-pyrrol-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4,4-dimethylpyrrolidin-1-yl)-7-azaspiro[3.5]nonan-7-yl)-N-((3-nitro-4-(((tetrahydro-2H-pyran-1-yl)methyl)amino)phenyl)sulfonyl)benzamide;
2-((1H-pyrrolo[2,3-b]pyridin-5-yl)oxy)-4-(2-(2-(2-cyclopropylphenyl)-4,4-difluoropyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4-(trifluoromethyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-(2-(2-(2-cyclopropylphenyl)-4-(dimethylamino)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-1-(2-(2-(2-cyclopropylphenyl)-4-(2-(dimethylamino)ethoxy)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-3,3-dimethylpyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-(((((1s,4s) or (1r,4r))-4-((dimethyl(oxo)-l6-sulfaneylidene)amino)cyclohexyl)methyl)amino)-3-nitrophenyl)sulfonyl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-(methyl(3-nitro-4-(((tetrahydro-2H-pyran-4-yl)methyl)amino)phenyl)(oxo)-l6-sulfaneylidene)benzamide;
2-((1H-pyrrolo[2,3-b]pyridin-5-yl)oxy)-N-((4-(((-3-oxabicyclo[3.1.0]hexan-6-yl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((4-(((4-hydroxy-4-(trifluoromethyl)cyclohexyl)methyl)amino)-3-nitrophenyl)sulfonyl)benzamide;
2-((1H-pyrrolo[2,3-b]pyridin-5-yl)oxy)-N-((4-((((1r,4r)-4-hydroxy-4-(trifluoromethyl)cyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-N-((4-((((1s,4s)-4-hydroxy-4-(trifluoromethyl)cyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-4-(2-(S)-2-(2-isopropylphenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)benzamide;
2-((1H-pyrrolo[2,3-b]pyridin-5-yl)oxy)-4-(2-((S)-2-(2-isopropylphenyl)pyrrolidin-1-yl)-7-azaspiro[3.5]nonan-7-yl)-N-((4-((((S)-4-methylcyclohex-3-en-1-yl)methyl)amino)-3-nitrophenyl)sulfonyl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-(prop-1-en-2-yl)phenyl)pyrrolidin-1-yl)-7-azaspiro[3.5]nonan-7-yl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-propylphenyl)pyrrolidin-1-yl)-7-azaspiro[3.5]nonan-7-yl)benzamide;
2-((1H-pyrrolo[2,3-b]pyridin-5-yl)oxy)-4-(6-((S)-2-(2-cyclopropylphenyl)pyrrolidin-1-yl)-2-azaspiro[3.3]heptan-2-yl)-N-((4-((((1r,4r)-1-hydroxy-4-methylcyclohexyl)methyl)amino)-3-nitrophenyl)sulfonyl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(6-((S)-2-(2-cyclopropylphenyl)pyrrolidin-1-yl)-2-azaspiro[3.3]heptan-2-yl)benzamide;
N-((4-((((R)-1,4-dioxan-2-yl)methyl)amino)-3-nitrophenyl)sulfonyl)-2-((1H-pyrrolo[2,3-b]pyridin-5-yl)oxy)-4-(6-((S)-2-(2-cyclopropylphenyl)pyrrolidin-1-yl)-2-azaspiro[3.3]heptan-2-yl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(6-((S)-2-(2-isopropylphenyl)pyrrolidin-1-yl)-2-azaspiro[3.3]heptan-2-yl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(6-((S)-2-(2-isopropylphenyl)pyrrolidin-1-yl)-2-azaspiro[3.3]heptan-2-yl)benzamide;
2-((1H-pyrrolo[2,3-b]pyridin-5-yl)oxy)-4-(6-((S)-2-(2-ethylphenyl)pyrrolidin-1-yl)-2-azaspiro[3.3]heptan-2-yl)-N-((4-((((1r,4r)-4-hydroxy-4-methylcyclohexyl)methyl)amino)-3-nitrophenyl)sulfonyl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(6-((S)-2-(2-ethylphenyl)pyrrolidin-1-yl)-2-azaspiro[3.3]heptan-2-yl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((3-nitro-4-((2-(tetrahydro-2H-pyran-4-yl)ethyl)amino)phenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((4-((2-morpholinoethyl)amino)-3-nitrophenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((3-nitro-4-((2-(3-oxomorpholino)ethyl)amino)phenyl)sulfonyl)benzamide;
2-((1H-pyrrolo[2,3-b]pyridin-5-yl)oxy)-N-((4-(((3-oxabicyclo[3.1.0]hexan-6-yl)methyl)amino)-3-nitrophenyl)sulfonyl)-4-(2-((S)-2-(2-cyclopropylphenyl)pyrrolidin-1-yl)-7-azaspiro[3.5]nonan-7-yl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-(((2,6-dimethyltetrahydro-2H-pyran-4-yl)methyl)amino)-3-nitrophenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((3-nitro-4-(((2,2,6,6-tetramethyltetrahydro-2H-pyran-4-yl)methyl)amino)phenyl)sulfonyl)benzamide:
2-((1H-pyrrolo[2,3-b]pyridin-5-yl)oxy)-4-(2-(S)-2-(2-cyclopropylphenyl)pyrrolidin-1-yl)-6-azaspiro[3.4]octan-6-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-(6-((S)-2-(2-cyclopropylphenyl)pyrrolidin-1-yl)-2-azaspiro[3.4]octan-2-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-((7R or 7S)-7-((S)-2-(2-cyclopropylphenyl)pyrrolidin-1-yl)-2-azaspiro[4.4]nonan-2-yl)-N-((4-((((1r,4r)-4-hydroxy-4-methyl cyclohexyl)methyl)amino)-3-nitrophenyl)sulfonyl)benzamide;
2-((1H-pyrrolo[2,3-b]pyridin-5-yl)oxy)-4-((7S or 7R)-7-((S)-2-(2-cyclopropylphenyl)pyrrolidin-1-yl)-2-azaspiro[4.4]nonan-2-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-(((2,2-dimethyltetrahydro-2H-pyran-4-yl)methyl)amino)-3-nitrophenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((4-(3-methyl-3-((tetrahydro-2H-pyran-4-yl)methyl)ureido)-3-nitrophenyl)sulfonyl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-phenylpyrrolidin-1-yl)-7-azaspiro[3.5]nonan-7-yl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-((((cis or trans)-4-hydroxytetrahydrofuran-2-yl)methyl)amino)-3-nitrophenyl)sulfonyl)benzamide; and
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-((((trans or cis)-4-hydroxytetrahydrofuran-2-yl)methyl)amino)-3-nitrophenyl)sulfonyl)benzamide;
or a pharmaceutically acceptable salt thereof, or a stereoisomer thereof.

In some embodiments, the Bcl-2 inhibitor in present disclosure is 2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide (Compound 1) or a pharmaceutically acceptable salt thereof.

### Preparation of Bcl-2 Inhibitors

All the Bcl-2 inhibitors having Formulas (III-B), (III-C), (III-D) or (III-E), including 2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide (Compound 1), can be prepared by the method disclosed in international publication WO2019/210828A1.

### Preparation of Compound 1

### Step 1: 2,2-dimethoxy-7-azaspiro[3.5]nonane hydrochloride

To the solution of tert-butyl 2-oxo-7-azaspiro[3.5]nonane-7-carboxylate (500 g, 2.09 mol) in MeOH (750 mL) and EA (750 mL) was added conc. HCl acid (350 mL, 4.18 mol) at room temperature and stirred for 4 hours. After concentrated in vacuum, MeOH (750 mL) was added into the residue and then the resulting mixture was concentrated in vacuum (repeated this work-up twice). The brown residue was suspended in EA (1250 mL) and stirred for 1 hour. The solid precipitation was filtered and dried in vacuum to afford the title product as an off-white powder (350 g, yield: 76.0%). ¹H NMR (400 MHz, DMSO-d₆) δ ppm: 3.03 (s, 6H), 2.96-2.89 (m, 4H), 1.93 (s, 4H), 1.74-1.67 (m, 4H). MS (ESI, m/e) [M+1]⁺ 186.0.

### Step 2: methyl 2-((1H-pyrrolo[2,3-b]pyridin-5 yl)oxy)-4-(2,2-dimethoxy-7-azaspiro[3.5]nonan-7-yl)benzoate

The mixture of methyl 2-((1H-pyrrolo[2,3-b]pyridin-5-yl)oxy)-4-fluorobenzoate (100 g), 2,2-dimethoxy-7-azaspiro[3.5]nonane hydrochloride (116 g, 1.5 eq.) and DBU (160 g, 3.0 eq.) in NMP (500 mL) was stirred for 16 hours at 85° C. After the reaction was completed, the mixture was cooled to 50±5° C. and citric acid in water (2%, 5 L) was added drop-wise into the system under stirring. After filtered, the cake was collected and dissolved with DCM (1.5 L). The solution of crude product was washed with citric acid in water (2%, 1.5 L), saturated aq. NaHCO₃ (1.5 L) and 15% aq. NaCl (1.5 L), and then dried over anhydrous Na₂SO₄. Silica gel (100 g) was added into the solution of the crude product under stirring and then filtered. The filtrate was concentrated to 300 mL. MTBE (500 mL) was poured into the system. After stirred for 2 hours, the cake was collected after filtration and was dried in vacuum to give an off-white solid (192 g, yield: 72.1%). ¹H NMR (400 MHz, DMSO-d₆) δ ppm: 11.63 (s, 1H), 8.00 (d, J=2.4 Hz, 1H), 7.76 (d, J=9.2 Hz, 1H), 7.47 (t, J=3.2 Hz, 1H), 7.42 (d, J=2.4 Hz, 1H), 6.79 (dd, J=2.4 Hz, J=9.2 Hz, 1H), 6.39-6.36 (m, 2H), 3.64 (s, 3H), 3.17-3.12 (m, 4H), 3.01 (s, 6H), 1.86 (s, 4H), 1.54-1.50 (m, 4H). MS (ESI, m/e) [M+1]⁺ 451.9.

### Step 3: methyl 2-((1H-pyrrolo[2,3-b]pyridin-5-yl)oxy)-4-(2-oxo-7-azaspiro[3.5]nonan-7-yl)benzoate

To the solution of methyl 2-((1H-pyrrolo[2,3-b]pyridin-5-yl)oxy)-4-(2,2-dimethoxy-7-azaspiro[3.5]nonan-7-yl)benzoate (176 g, 0.39 mol) in DCM (2 L) was added diluted HCl acid (1M, 1.5 L) and stirred for overnight. After the reaction was completed, the mixture was cooled to 10° C. and was adjusted to pH=8-9 with aqueous NaOH solution (4 M) under stirring. The organic phase was separated and washed with 15% aq. NaCl (1 L), then washed with H₂O (1 L). After the organic phase was concentrated to 500 mL, MTBE (1 L) was poured into the solution and then the system was concentrated to 500 mL (repeated this work-up 3 times). The resulting system was stirred for 0.5 hour. After filtration, the cake was collected and then dried in vacuum to obtain the tittle product as a white solid (152 g. yield: 96.23%). ¹H NMR (400 MHz, DMSO-d₆) δ ppm: 11.64 (s, 1H), 8.02 (d, J=2.4 Hz, 1H), 7.78 (d, J=9.2 Hz, 1H), 7.47 (t, J=3.2 Hz, 1H), 7.44 (d, J=2.4 Hz, 1H), 6.83 (dd, J=2.4 Hz, J=9.2 Hz, 1H), 6.43 (d, J=2.4 Hz, 1H), 6.38-6.36 (m, 1H), 3.65 (s, 3H), 3.24-3.21 (m, 4H), 2.80 (s, 4H), 1.70-1.67 (m, 4H). MS (ESI, m/e) [M+1]⁺ 405.9.

### Step 4: (5)-tert-butyl 2-(2-(prop-1-en-2-yl)phenyl)pyrrolidine-1-carboxylate

To a mixture of (S)-tert-butyl 2-(2-bromophenyl)pyrrolidine-1-carboxylate (50 g, 153.3 mmol) and 4,4,5,5-tetramethyl-2-(prop-1-en-2-yl)-1,3,2-dioxaborolane (38.6 g, 229.9 mmol) in dioxane (500 mL) and H₂O (50 mL) was added Cs₂CO₃ (100 g, 305 mmol) and Pd(dppf)Cl₂ (6.6 g, 7.5 mmol). The mixture was stirred at 100° C. for 8 hours. TLC showed the reaction was completed. The mixture was concentrated in vacuum. The residue was purified by column chromatography on silica gel (eluent: PE/EA (v/v)=100/1 to 10/1) to obtain (S)-tert-butyl 2-(2-(prop-1-en-2-yl)phenyl)pyrrolidine-1-carboxylate (65 g, crude). The crude product was used directly in next step.

### Step 5: (S)-tert-butyl 2-(2-isopropylphenyl)pyrrolidine-1-carboxylate

To a solution of (S)-tert-butyl 2-(2-(prop-1-en-2-yl)phenyl)pyrrolidine-1-carboxylate (30 g, 104.39 mmol) in MeOH (500 mL) was added Pd/C (10 g, 10%) and the mixture was stirred at 20° C. under H₂ (15 Psi) for 12 hours. TLC showed the reaction was completed. The mixture was filtered and the filtrate was concentrated in vacuum to give (S)-tort-butyl 2-(2-isopropylphenyl)pyrrolidine-1-carboxylate (60 g, crude), which was used in next step without further purification. ¹H NMR (400 MHz, CDCl₃) δ ppm: 7.39-6.90 (m, 4H), 5.36-5.04 (m, 1H), 3.77-3.52 (m, 2H), 3.20-3.17 (m, 1H), 2.47-2.24 (m, 1H), 1.96-1.65 (m, 3H), 1.54-1.38 (m, 2H), 1.31-1.22 (m, 8H), 1.17 (s, 7H).

### Step 6: (S)-2-(2-isopropylphenyl)pyrrolidine hydrochloride

To a solution often-butyl 2-(2-isopropylphenyl)pyrrolidine-1-carboxylate (55 g, 190 mmol) in DCM (50 mL) was added HCl in 1,4-dioxane (4 M, 142 mL, 570 mmol) dropwise at room temperature. The mixture was stirred at room temperature for overnight. The mixture was concentrated in vacuum. The resulting residue was slurried with EA (100 mL) and then filtered, dried in vacuum to give (S)-2-(2-isopropylphenyl)pyrrolidine hydrochloride 26 g (yield: 60.4%). ¹H NMR (400 MHz, DMSO-d₆) δ ppm: 9.93 (s, 1H), 8.81 (s, 1H), 7.63-7.57 (m, 1H), 7.41-7.34 (m, 2H), 7.32-7.24 (m, 1H), 4.91-4.75 (m, 1H), 3.47-3.35 (m, 1H), 3.31-3.25 (m, 1H), 2.40-2.21 (m, 1H), 2.19-1.86 (m, 3H), 1.25 (d, J=6.7 Hz, 3H), 1.17 (d, J=6.7 Hz, 3H). MS (ESI, m/e) [M+1]⁺ 190.0.

### Step 7: methyl (S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoate

A mixture of (S)-2-(2-isopropylphenyl)pyrrolidine hydrochloride (120 g, 0.535 mole) and methyl 2-((1H-pyrrolo[2,3-b]pyridin-5-yl)oxy)-4-(2-oxo-7-azaspiro[3.5]nonan-7-yl)benzoate (218 g, 0.509 mole) in DCM (2.2 L) was charged into a reactor. The temperature was controlled blow 30° C. and NaBH(OAc)₃ (216 g, 1.018 mole) was added into the reactor in 5-6 portions. Then the reaction mixture was stirred at room temperature and monitored by TLC. After the starting material ketone was consumed completely, the mixture was adjusted to pH=4˜5 with diluted HCl acid (0.5 M). The separated organic phase was washed with H₂O (600 mL×2) and then washed with aq. NaHCO₃ (600 mL×2), saturated aq. NaCl (600 mL). The organic phase was collected, then dried over anhydrous Na₂SO₄ and concentrated. 256 g off-white solid was obtained as the crude product, which was used in the next step directly. MS (ESI, m/e) [M+1]⁺ 579.0.

### Step 8: (S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoic acid

To a solution of methyl (S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoate (105 g, 181.7 mmol) in THF (525 mL) and MeOH (525 mL) was added aq. NaOH (3.5 M). It was stirred at room temperature overnight. After THF and MeOH were removed in vacuum, 3.5 L of water was added into the residue. The resulting mixture was adjusted to pH=5˜6 with 3 N HCl acid at room temperature with stirring. The precipitate was filtered and dried in vacuum to give the product as a white solid (102.4 g, yield: 99%). ¹H NMR (400 MHz, DMSO-d₆) δ ppm: 12.13 (s, 1H), 11.58 (s, 1H), 7.95 (s, 1H), 7.67 (d, J=8.0 Hz, 1H), 7.56-7.40 (m, 2H), 7.35 (s, 1H), 7.27-7.04 (m, 3H), 6.68 (d, J=8.0 Hz, 1H), 6.32 (s, 2H), 3.62 (s, 1H), 3.32-3.26 (m, 1H), 3.10-3.04 (m, 4H), 2.35-2.30 (m, 1H), 2.9-2.15 (m, 1H), 1.74-1.64 (m, 4H), 1.52-1.37 (m, 6H), 1.28-1.06 (m, 6H). MS (ESI, m/e) [M+1]⁺ 564.9.

### Step 9: 2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide

A mixture of (S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoic acid (44 g, 78 mmol), 4-((((1r,4r)-4-hydroxy-4-methylcyclohexyl)methyl)amino)-3-nitrobenzenesulfonamide (26.8 g, 78 mmol), TEA (15.7 g, 156 mmol), EDCI (19.4 g, 101 mmol) and DMAP (19 g, 156 mmol) in anhydrous DCM (880 mL) was stirred overnight at room temperature. The reaction was monitored by HPLC. After starting material of (S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoic acid was consumed completely, the reaction mixture was heated to ˜35° C. and N¹,N¹-dimethylethane-1,2-diamine (17.2 g, 195 mmol) was added in one portion. The reaction was stirred for another 12 hours. The mixture was washed twice with 10 wt % aq. AcOH solution (300 mL×2) and then washed with saturated aq. NaHCO₃ (300 mL×2). The organic layer was collected and concentrated to about 90 mL. 22 g of silica gel was added and stirred for 2 hours. After filtration, 180 mL EA was added into the filtrate at reflux and further stirred for 5 hours. After the mixture was cooled to mom temperature, the precipitate was filtered and then the wet cake was washed twice with EA (180 mL). After drying in vacuum at 80-90° C., the desired compound was obtained (48 g, yield: 69.5%). ¹H NMR (DMSO-d₆) δ ppm: 11.65 (s, 1H), 11.11 (br, 1H), 8.58-8.39 (m, 2H), 8.00 (d, J=2.8 Hz, 1H), 7.74 (d, J=8.8 Hz, 1H), 7.57-7.37 (m, 4H), 7.30-7.10 (m, 3H), 7.00 (d, J=9.2 Hz, 1H), 6.65 (d, J=1.2 Hz, 1H), 6.35 (s, 1H), 6.17 (s, 1H), 4.24 (s, 1H), 3.39-3.20 (m, 5H), 3.04-2.88 (m, 4H), 2.23 (s, 1H), 1.94-1.47 (m, 11H), 1.44-1.26 (m, 7H), 1.19 (d, J=8.0 Hz, 3H), 1.14 (d, J=8.0 Hz, 3H), 1.10 (s, 4H). MS (ESI, m/e) [M+1]⁺ 889.9.

### Methods of Treatment

In one aspect, the present disclosure provides a method of treating cancer. In certain aspects, the method comprises administering to a patient in need an effective amount of Compound 1. The cancer is B-cell malignancies, selected from the group consisting of non-Hodgkin lymphoma (NHL) expected to be at low risk of tumor lysis syndrome, low-tumor-burden chronic lymphocytic leukemia/small lymphocytic lymphoma (CLL/SLL), high-tumor-burden CLL/SLL, mantle cell lymphoma (MCL), and Waldenstrom macroglobulinemia (WM). In some embodiments, the B-cell malignancies is relapsed/refractory.

Compound 1 can be administered by any suitable means, including oral, parenteral, intrapulmonary, and intranasal, and, if desired for local treatment, intralesional administration. Dosing can be by any suitable route. Various dosing schedules including but not limited to single or multiple administrations over various time-points, bolus administration, and pulse infusion are contemplated herein.

Compound 1 would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. Compound 1 is optionally formulated with one or more agents currently used to prevent or treat the disorder in question. The effective amount of such other agents depends on the amount of Compound 1 in the formulation, the type of disorder or treatment, and other factors discussed above.

For the prevention or treatment of disease, the appropriate dosage of Compound 1 will depend on the type of disease to be treated, the severity and course of the disease, whether Compound 1 is administered for preventive or therapeutic purposes, previous therapy, the patient's clinical history and response to Compound 1, and the discretion of the attending physician. Compound 1 is suitably administered to the patient at one time or over a series of treatments.

### EXAMPLES

The present invention is further exemplified, but not limited to, by the following examples that illustrate the invention.

### Example 1: Efficacy Study of Bcl-2 Inhibitors in RS4;11 Acute Lymphoblastic Leukemia (ALL) Subcutaneous Xenograft Model

The RS4;11 cells are of acute lymphoblastic leukemia (ALL) origin and were obtained from American Type Culture Collection (ATCC CRL-1873, Manassas, VA, DC, USA). Cells were grown in RPM 1640 medium (Corning, Cat #10-040-CVR), supplemented with 10% (v/v) fetal bovine serum (Gibco, Cat #10099-141C), and 100 μg/mL of penicillin and streptomycin (Gibco, Cat #15140-122). RS4;11 cells were maintained as suspension cell cultures at 37° C. in a 5% CO₂ atmosphere. Five to six-week-old female NCG mice were purchased from Gempharmatech of Information Technology Center. All animals were maintained under specific pathogen free (SPF) “full barrier” condition with free access to food and water. Mice were group-housed under a 12 h light:dark cycle (lights on at 08:00 h), at a temperature of 20-26° C. and 37-62% humidity in IVC cages (Lingyunboji (Beijing) Technology Co., Ltd.). Mice were fed with complete granulated feed with Co60 radiosterilization (Beijing Ke Ao Xie Li Feed Co., Ltd.).

On the day of implantation, RS4;11 cells were harvested and re-suspended with appropriate volume of ice cold DPBS and same volume of Matrigel (Corning, Cat #356237) to give a final concentration of 5×10⁷ cells/mL. Re-suspended cells were placed on ice prior to inoculation. The right front flank region of each mouse was cleaned with 75% ethanol prior to cell inoculation. Each animal was injected subcutaneously with 1×10⁷ cells in 200 μL of cell suspension in the right front flank via a 26-gauge needle. After implantation, primary tumor volume was measured in two dimensions using a caliper.

Animals were randomly assigned into 10 groups with 10 mice per group according to body weight and tumor volume (100 mm³-200 mm³). The groups consisted of vehicle group, 5, 15, 50 mg/kg of venetoclax with QD dosing, 5, 15, 50 mg/kg of Compound 1 with QD dosing, and 2.5, 7.5, 25 mg/kg of Compound 1 with BID dosing. Treatments were administered by oral gavage (p.o.) in a volume of 10 mL/kg body weight. Body weight was assessed immediately before dosing and volume dosed was adjusted accordingly.

Individual body weight was recorded twice weekly, with mice being monitored daily for clinical signs of toxicity for the duration of the study. Mice were euthanized using carbon dioxide when their tumor volume reached 2,000 mm³, the tumor was ulcerated, or body weight loss exceeded 20%.

Tumor volume was calculated using the formula: V=0.5×(a×b²) where a and b are the long and short diameters of the tumor, respectively.

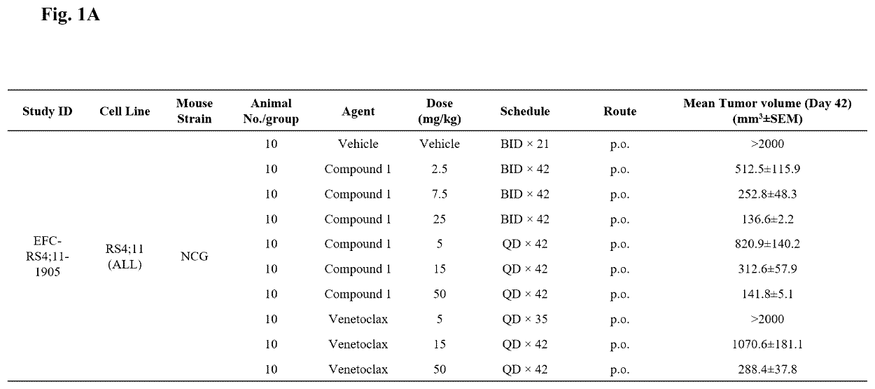

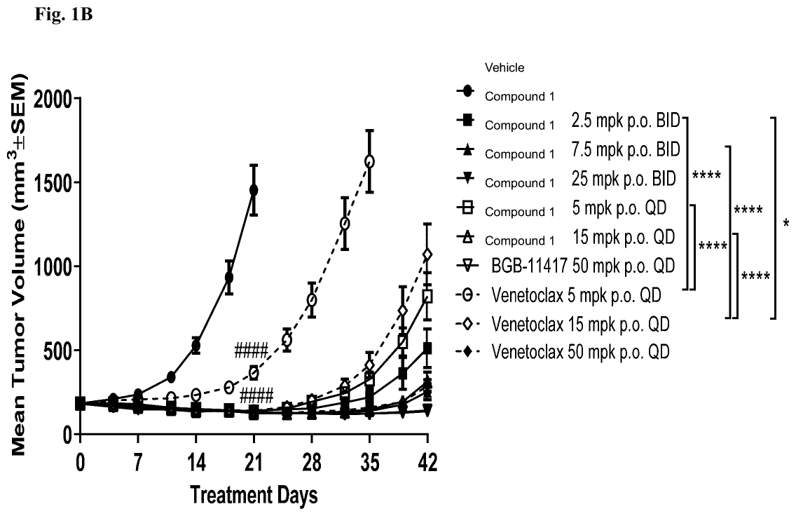

In vivo efficacy of Compound 1 was examined and compared to venetoclax in RS4;11 ALL xenografts grown subcutaneously in NCG mice. Following oral administration at well tolerated doses, Compound 1 potently and dose-dependently inhibited tumor growth (FIGS. 1A-1B and Table 1). At the same total daily doses of 5 and 15 mg/kg, Compound 1 demonstrated significantly better efficacy when compared with venetoclax. Venetoclax was dosed at 400 mg daily in clinic. Its clinically relevant dose in mice is around 15 mg/kg QD based on free AUC. Compound 1 2.5 mg/kg BID was more active than venetoclax at 15 mg/kg QD. Furthermore, at the same total daily doses of 15 and 50 mg/kg, Compound 1 QD and BID dosing schedule showed equivalent anti-tumor activities. These results are shown in FIGS. 1A-1B.

All treatment groups had no significant impact on animal body weight throughout the study.

TABLE 1

| | | | | Mean Tumor |
|---|---|---|---|---|
| | Dose | | | volume (Day 42) |
| Agent | (mg/kg) | Schedule | Route | (mm³ ± SEM) |
| Vehicle | Vehicle | BID × 21 | p.o. | >2000 |
| Compound 1 | 2.5 | BID × 42 | p.o. | 512.5 ± 115.9 |
| Compound 1 | 7.5 | BID × 42 | p.o. | 252.8 ± 48.3 |
| Compound 1 | 25 | BID × 42 | p.o. | 136.6 ± 2.2 |
| Compound 1 | 5 | QD × 42 | p.o. | 820.9 ± 140.2 |
| Compound 1 | 15 | QD × 42 | p.o. | 312.6 ± 57.9 |
| Compound 1 | 50 | QD × 42 | p.o. | 141.8 ± 5.1 |
| Venetoclax | 5 | QD × 35 | p.o. | >2000 |
| Venetoclax | 15 | QD × 42 | p.o. | 1070.6 ± 181.1 |
| Venetoclax | 50 | QD × 42 | p.o. | 288.4 ± 37.8 |

### Example 2: Efficacy Study of Bcl-2 Inhibitors in MAVER-1 Mantle Cell Lymphoma (MCL) Subcutaneous Xenograft Model

MAVER-1 cells are of mantle cell lymphoma (MCL) origin and were obtained from American Type Culture Collection (ATCC CRL-3008, Manassas, VA, DC, USA). Cells were grown in RPM 1640 medium (Corning, Cat #10-040-CVR), supplemented with 10% (v/v) fetal bovine serum (Gibco, Cat #10099-141C), and 100 μg/mL of penicillin and streptomycin (Gibco, Cat #15140-122). MAVER-1 cells were maintained as suspension cell cultures at 37° C. in a 5% CO₂ atmosphere. Five to six-week-old female NCG mice were purchased from Gempharmatech of Information Technology Center. All animals were maintained under specific pathogen free (SPF) “full barrier” condition with free access to food and water. Mice were group-housed under a 12 h light:dark cycle (lights on at 08:00 h), at a temperature of 21-26° C. and 44-61% humidity in IVC cages (Lingyunboji (Beijing) Technology Co., Ltd.). Mice were fed with complete granulated feed with Co60 radiosterilization (Beijing Ke Ao Xie Li Feed Co., Ltd.).

On the day of implantation, MAVER-1 cells were harvested and re-suspended with appropriate volume of ice cold DPBS and same volume of Matrigel (Corning, Cat #356237) to give a final concentration of 1.5×10⁷ cells/mL. Re-suspended cells were placed on ice prior to inoculation. The right front flank region of each mouse was cleaned with 75% ethanol prior to cell inoculation. Each animal was injected subcutaneously with 3×10⁶ cells in 200 μL of cell suspension in the right front flank via a 26-gauge needle. After implantation, primary tumor volume was measured in two dimensions using a caliper.

Animals were randomly assigned into 7 groups with 10 mice per group according to body weight and tumor volume (100 mm³-200 mm³). The groups consisted of vehicle group, 5, 15 mg/kg of venetoclax with QD dosing, 5, 15 mg/kg of Compound 1 with QD dosing, and 2.5, 7.5 mg/kg of Compound 1 with BID dosing. Treatments were administered by oral gavage (p.o.) in a volume of 10 mL/kg body weight. Body weight was assessed immediately before dosing and volume dosed was adjusted accordingly.

Individual body weight was recorded twice weekly, with mice being monitored daily for clinical signs of toxicity for the duration of the study. Mice were euthanized using carbon dioxide when their tumor volume reached 2,000 mm³, the tumor was ulcerated, or body weight loss exceeded 20%.

Tumor volume was calculated using the formula: V=0.5×(a×b²) where a and b are the long and short diameters of the tumor, respectively. Tumor growth inhibition (TGI) was calculated using the following formula: % TGI=100×[1−(treatedₜ−treatedₜ₀)/(vehicleₜ−vehicleₜ₀)] (treated t=treated tumor volume at time t, treated t0=treated tumor volume at time 0, vehicle t=vehicle tumor volume at time t and vehicle t0=vehicle tumor volume at time 0)

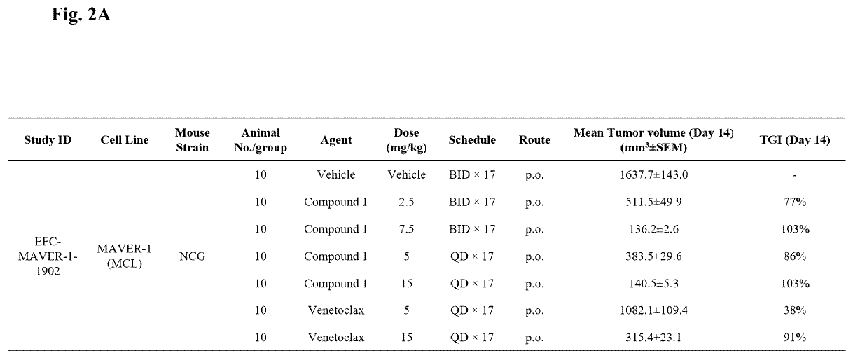

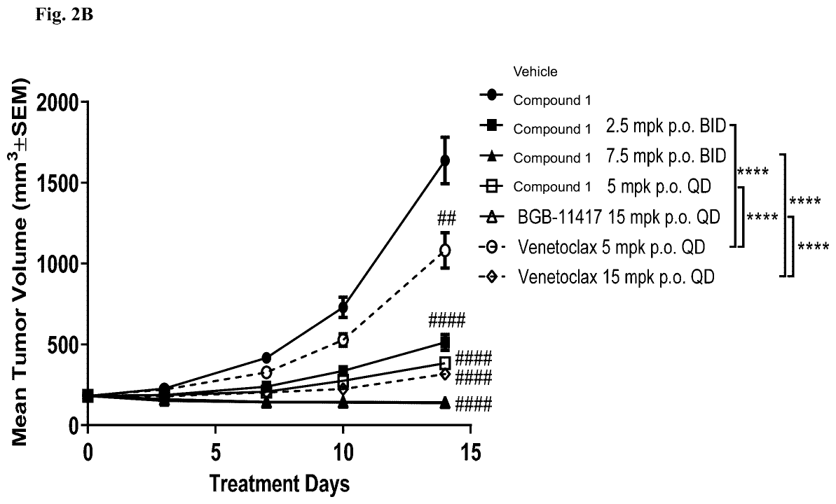

In vivo efficacy of Compound 1 was also examined and compared to venetoclax in MAVER-1 MCL xenografts grown subcutaneously in NCG mice. Compound 1 potently suppressed tumor growth in a dose-dependent manner. The tumor growth inhibition (TGI) on day 14 for 2.5, 7.5 mg/kg BID and 5, 15 mg/kg QD of Compound 1 were 77%, 103% and 86%, 103%, respectively. Venetoclax at 5 and 15 mg/kg QD achieved 38% and 91% TGI, respectively. At the same total daily doses of 5 and 15 mg/kg, Compound 1 showed more anti-tumor activities relative to venetoclax. Compound 1 15 mg/kg QD and 7.5 mg/kg BID were similarly active. These results are shown in FIGS. 2A-2B and Table 2.

All treatment groups had no significant impact on animal body weight throughout the study.

TABLE 2

| | | | | Mean Tumor | |
|---|---|---|---|---|---|
| | Dose | | | volume (Day 14) | TGI |
| Agent | (mg/kg) | Schedule | Route | (mm³ ± SEM) | (Day 14) |
| Vehicle | Vehicle | BID × 17 | p.o. | 1637.7 ± 143.0 | — |
| Compound 1 | 2.5 | BID × 17 | p.o. | 511.5 ± 49.9 | 77% |
| Compound 1 | 7.5 | BID × 17 | p.o. | 136.2 ± 2.6 | 103% |
| Compound 1 | 5 | QD × 17 | p.o. | 383.5 ± 29.6 | 86% |
| Compound 1 | 15 | QD × 17 | p.o. | 140.5 ± 5.3 | 103% |
| Venetoclax | 5 | QD × 17 | p.o. | 1082.1 ± 109.4 | 38% |
| Venetoclax | 15 | QD × 17 | p.o. | 315.4 ± 23.1 | 91% |

### Example 3: Efficacy Study of Bcl-2 Inhibitors in Toledo Diffuse Large B Cell Lymphoma (DLBCL) Subcutaneous Xenograft Model

Toledo cells are of diffuse large B cell lymphoma (DLBCL) origin and were obtained from American Type Culture Collection (ATCC CRL-2631, Manassas, VA, DC, USA). Cells were grown in RPMI 1640 medium (Corning. Cat #10-040-CVR), supplemented with 10% (v/v) fetal bovine serum (Gibco, Cat #10099-141C), and 100 μg/mL of penicillin and streptomycin (Gibco, Cat #15140-122). Toledo cells were maintained as suspension cell cultures at 37° C. in a 5% CO₂ atmosphere. Five to six-week-old female NCG mice were purchased from Gempharmatech of Information Technology Center. All animals were maintained under specific pathogen free (SPF) “full barrier” condition with free access to food and water. Mice were group-housed under a 12 h light:dark cycle (lights on at 08:00 h), at a temperature of 21-26° C. and 35-61% humidity in IVC cages (Lingyunboji (Beijing) Technology Co., Ltd.). Mice were fed with complete granulated feed with Co60 radiosterilization (Beijing Ke Ao Xie Li Feed Co., Ltd.).

On the day of implantation, Toledo cells were harvested and re-suspended with appropriate volume of ice cold DPBS and same volume of Matrigel (Corning, Cat #356237) to give a final concentration of 1.5×10⁷ cells/mL. Re-suspended cells were placed on ice prior to inoculation. The right front flank region of each mouse was cleaned with 75% ethanol prior to cell inoculation. Each animal was injected subcutaneously with 3×10⁶ cells in 200 μL of cell suspension in the right front flank via a 26-gauge needle. After implantation, primary tumor volume was measured in two dimensions using a caliper.

Transplanted animals were randomized into 10 groups with 10 mice per group on day 0 according to transplantation sequence and body weight. The groups consisted of vehicle group, 5, 15, 50 mg/kg of venetoclax with QD dosing, 5, 15, 50 mg/kg of Compound 1 with QD dosing, and 2.5, 7.5, 25 mg/kg of Compound 1 with BID dosing. Treatments were administered by oral gavage (p.o.) in a volume of 10 mL/kg body weight. Body weight was assessed immediately before dosing and volume dosed was adjusted accordingly.

Individual body weight was recorded twice weekly, with mice being monitored daily for clinical signs of toxicity for the duration of the study. Mice were euthanized using carbon dioxide when their tumor volume reached 2,000 mm³, the tumor was ulcerated, or body weight loss exceeded 20%.

Tumor volume was calculated using the formula: V=0.5×(a×b²) where a and b are the long and short diameters of the tumor, respectively. Tumor growth inhibition (TGI) was calculated using the following formula: % TGI=100×[1−(treatedₜ−treatedₜ₀)/(vehicleₜ−vehicleₜ₀)] (treated t=treated tumor volume at time t, treated t0=treated tumor volume at time 0, vehicle t=vehicle tumor volume at time t and vehicle t0=vehicle tumor volume at time 0)

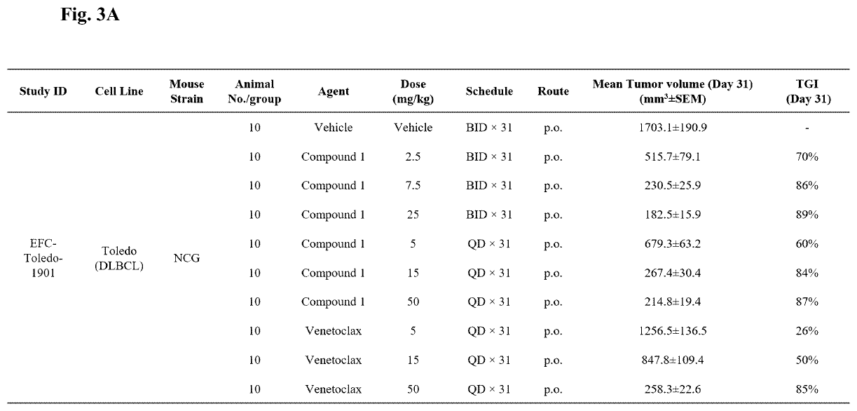

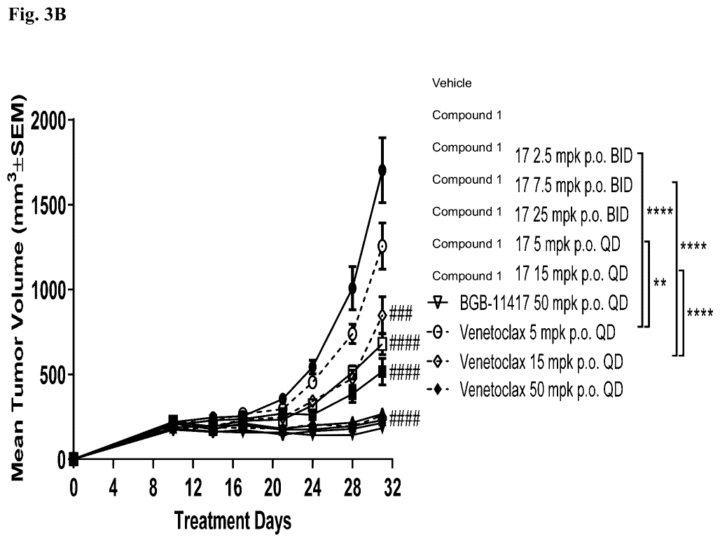

In vivo efficacy of Compound 1 was further examined and compared to venetoclax in Toledo DLBCL subcutaneous xenograft model. Following daily oral administration at well tolerated doses at 2.5, 7.5, 25 mg/kg BID or 5, 15, 50 mg/kg QD, Compound 1 induced dose-dependent anti-tumor effects. At the same total daily doses of 5 and 15 mg/kg, Compound 1 demonstrated significantly better efficacy when compared with venetoclax. These results are shown in FIGS. 3A-3B and Table 3.

All treatment groups had no significant impact on animal body weight throughout the study.

TABLE 3

| | | | | Mean Tumor | |
|---|---|---|---|---|---|
| | Dose | | | volume (Day 31) | TGI |
| Agent | (mg/kg) | Schedule | Route | (mm³ ± SEM) | (Day 31) |
| Vehicle | Vehicle | BID × 31 | p.o. | 1703.1 ± 190.9 | — |
| Compound 1 | 2.5 | BID × 31 | p.o. | 515.7 ± 79.1 | 70% |
| Compound 1 | 7.5 | BID × 31 | p.o. | 230.5 ± 25.9 | 86% |
| Compound 1 | 25 | BID × 31 | p.o. | 182.5 ± 15.9 | 89% |
| Compound 1 | 5 | QD × 31 | p.o. | 679.3 ± 63.2 | 60% |
| Compound 1 | 15 | QD × 31 | p.o. | 267.4 ± 30.4 | 84% |
| Compound 1 | 50 | QD × 31 | p.o. | 214.8 ± 19.4 | 87% |
| Venetoclax | 5 | QD × 31 | p.o. | 1256.5 ± 136.5 | 26% |
| Venetoclax | 15 | QD × 31 | p.o. | 847.8 ± 109.4 | 50% |
| Venetoclax | 50 | QD × 31 | p.o. | 258.3 ± 22.6 | 85% |

### Example 4: Efficacy Study of Bcl-2 Inhibitors in RS4;11 Bcl-2G101V KI Acute Lymphoblastic Leukemia (ALL) Subcutaneous Xenograft Model

The RS4;11 Bcl-2G101V KI cells are of acute lymphoblastic leukemia (ALL) origin and were screened in house. Cells were grown in RPMI 1640 medium (Corning, Cat #10-040-CVR), supplemented with 10% (v/v) fetal bovine serum (Gibco, Cat #10099-141C), and 100 μg/mL of penicillin and streptomycin (Gibco, Cat #15140-122). RS4;11 Bcl-2G101V KI cells were maintained as suspension cell cultures at 37° C. in a 5% CO₂ atmosphere. Five to six-week-old female NCG mice were supplied by GemPharmatech Co., Ltd, Jiangsu, China. All animals were maintained under specific pathogen free (SPF) “full barrier” condition with free access to food and water. Mice were group-housed under a 12 h light:dark cycle (lights on at 08:00 h), at a temperature of 20-26° C. and 37-62% humidity in WC cages (Lingyunboji (Beijing) Technology Co., Ltd.). Mice were fed with complete granulated feed with Co60 radio sterilization (Beijing Ke Ao Xie Li Feed Co., Ltd.).

On the day of implantation, RS4;11 Bcl-2G101V KI cells were harvested and re suspended with appropriate volume of ice cold DPBS and same volume of Matrigel (Corning, Cat #356237) to give a final concentration of 5×10⁷ cells/mL. Re-suspended cells were placed on ice prior to inoculation. The right front flank region of each mouse was cleaned with 75% ethanol prior to cell inoculation. Each animal was injected subcutaneously with 1×10⁷ cells in 200 μL of cell suspension in the right front flank via a 26-gauge needle. After implantation, primary tumor volume was measured in two dimensions using a caliper.

Animals were randomly assigned into 7 groups with 8 mice per group according to body weight and tumor volume (around 300 mm³). The groups consisted of vehicle group, 15, 50 and 100 mg/kg of venetoclax with QD dosing, 15, 50 and 100 mg/kg of Compound 1 with QD dosing. Treatments were administered by oral gavage (p.o.) in a volume of 10 mg/kg body weight. Body weight was assessed immediately before dosing and volume dosed was adjusted accordingly.

Individual body weight was recorded twice weekly, with mice being monitored daily for clinical signs of toxicity for the duration of the study. Mice were euthanized using carbon dioxide when their tumor volume reached 2,000 mm³, the tumor was ulcerated, or body weight loss exceeded 20%.

Tumor volume was calculated using the formula: V=0.5×(a×b²) where a and b are the long and short diameters of the tumor, respectively.

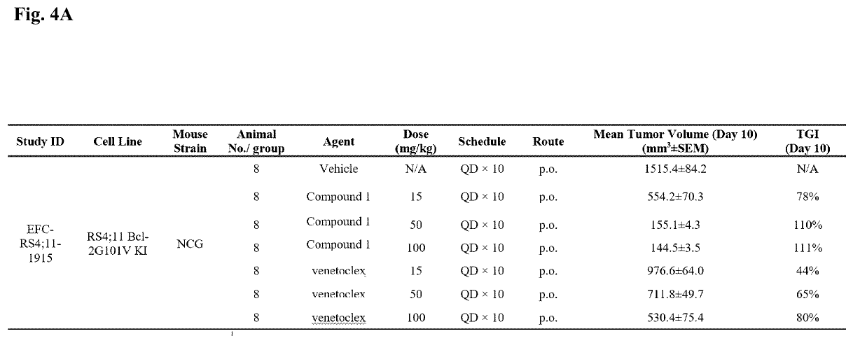

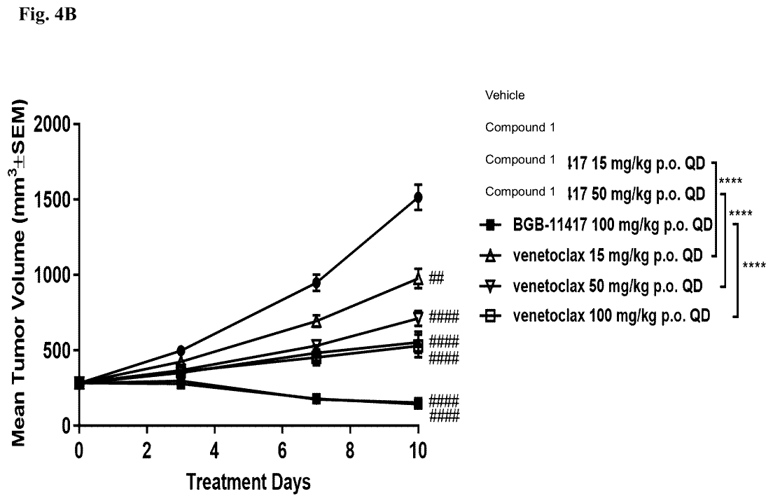

In vivo efficacy of Compound 1 was examined and compared to venetoclax in RS4;11 Bcl-2G101V KI xenografts grown subcutaneously in NCG mice. Venetoclax showed marginal efficacy even at higher dose level, whereas Compound 1 potently and dose-dependently inhibited tumor growth. These results are shown in FIGS. 4A-4B and Table 4. The curves for Compound 1 at 50 mg/kg p.o. QD and 100 mg/kg p.o. QD merged.

All treatment groups had no significant impact on animal body weight throughout the study.

TABLE 4

| | | | | Mean Tumor | |
|---|---|---|---|---|---|
| | Dose | | | Volume (Day 10) | TGI |
| Agent | (mg/kg) | Schedule | Route | (mm³ ± SEM) | (Day 10) |
| Vehicle | N/A | QD × 10 | p.o. | 1515.4 ± 84.2 | N/A |
| Compound 1 | 15 | QD × 10 | p.o. | 554.2 ± 70.3 | 78% |
| Compound 1 | 50 | QD × 10 | p.o. | 155.1 ± 4.3 | 110% |
| Compound 1 | 100 | QD × 10 | p.o. | 144.5 ± 3.5 | 111% |
| Venetoclax | 15 | QD × 10 | p.o. | 976.6 ± 64.0 | 44% |
| Venetoclax | 50 | QD × 10 | p.o. | 711.8 ± 49.7 | 65% |
| Venetoclax | 100 | QD × 10 | p.o. | 530.4 ± 75.4 | 80% |

### Example 5: Efficacy Evaluation of Bcl-2 Inhibitors in Combination with BTK Inhibitors in JeKo-1 Human Mantle Cell Lymphoma (MCL) Subcutaneous Xenograft Model

The JeKo-1 cells are of mantle cell lymphoma (MCL) origin and were obtained from American Type Culture Collection (ATCC, CRL-3006, Manassas, VA, DC, USA). Cells were grown in RPMI 1640 medium (Corning, Cat #10-040-CVR), supplemented with 10% (v/v) fetal bovine serum (Gibco, Cat #10099-141C), and 100 μg/mL of penicillin and streptomycin (Gibco, Cat #15140-122). JeKo-1 cells were maintained as suspension cell cultures at 37° C. in a 5% CO₂ atmosphere. Five to six-week-old female NCG mice were purchased from Gempharmatech of Information Technology Center. All animals were maintained under specific pathogen free (SPF) “full barrier” condition with free access to food and water. Mice were group-housed under a 12 h light:dark cycle (lights on at 08:00 h), at a temperature of 23-27° C. and 28-51% humidity in IVC cages (Lingyunboji (Beijing) Technology Co., Ltd.). Mice were fed with complete granulated feed with Co60 radiosterilization (Beijing Ke Ao Xie Li Feed Co., Ltd.). All experiments were carried out in accordance with BeiGene's IACUC.

On the day of implantation, JeKo-1 cells were harvested and re-suspended with appropriate volume of ice cold PBS and same volume of Matrigel (Corning, Cat #356237) to give a final concentration of 5×10⁷ cells/mL. Re-suspended cells were placed on ice prior to inoculation. The right front flank region of each mouse was cleaned with 75% ethanol prior to cell inoculation. Each animal was injected subcutaneously with 1×10⁷ cells in 200 μL of cell suspension in the right front flank via a 26-gauge needle. After implantation, primary tumor volume was measured in two dimensions using a caliper.

Transplanted animals were randomized into 8 groups with 10 mice per group on day 0 according to transplantation sequence and body weight. The groups consisted of vehicle group, 5, 15, 50 mg/kg of Compound 1 (a Bcl-2 inhibitor) with QD dosing, and 20 mg/kg of Compound B (a BTK inhibitor, Zanubrutinib, (S)-7-(1-acryloylpiperidin-4-yl)-2-(4-phenoxyphenyl)-4,5,6,7-tetra-hydropyrazolo[1,5-a]pyrimidine-3-carboxamide) with BID dosing, and their combinations. Treatments were administered by oral gavage (p.o.) in a volume of 10 mL/kg body weight. Body weight was assessed immediately before dosing and volume dosed was adjusted accordingly.

Individual body weight was recorded twice weekly, with mice being monitored daily for clinical signs of toxicity for the duration of the study. Mice were euthanized using carbon dioxide when their tumor volume reached 2,000 mm³, the tumor was ulcerated, or body weight loss exceeded 20%.

Tumor volume (TV) was calculated using the formula: TV=0.5×(a×b²) where a and b are the long and short diameters of the tumor, respectively. Tumor growth inhibition (TGI) was calculated using the following formula: % TGI=100×[1−(treatedₜ)/(vehicleₜ)] (treated t=treated tumor volume at time t, vehicle t=vehicle tumor volume at time t)

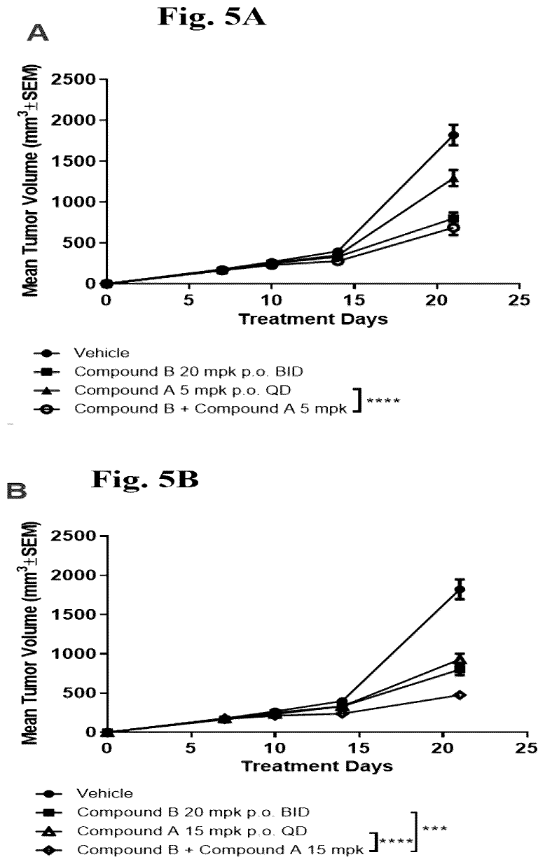

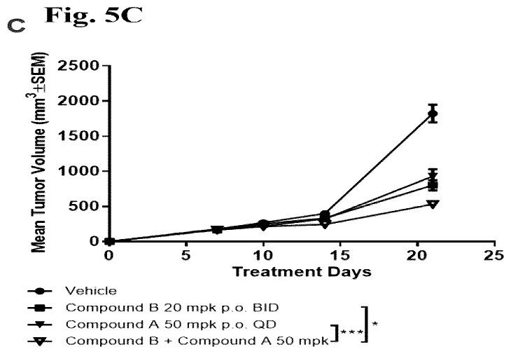

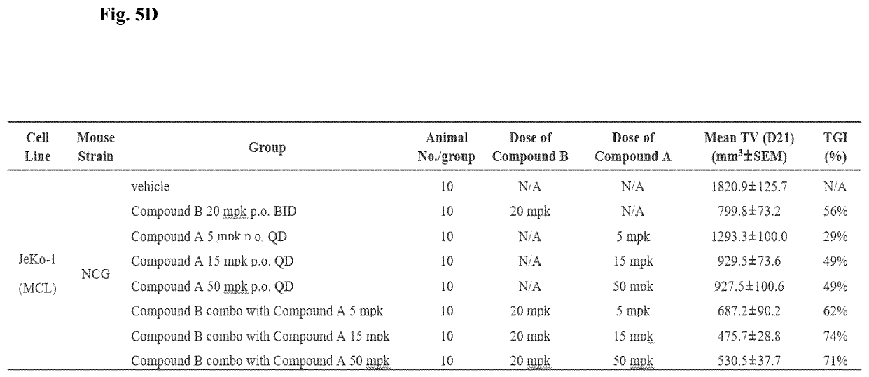

The in vivo efficacy of Compound 1 and Compound B was examined in JeKo-1 MCL subcutaneous xenograft model grown subcutaneously in NCG mice. Results are shown in FIGS. 5A, 5B, 5C and 5D. On day 21, Compound 1 at 5, 15 and 50 mg/kg QD, and Compound B at 20 mg/kg BID resulted in 29%, 49%, 49% and 56% of tumor growth inhibition (TGI), respectively. Combinations of Compound B at 20 mg/kg BID with Compound 1 at 5, 15 or 50 mpk QD resulted in 62%, 74% and 71% of TGI, respectively (Sec Table 1). The combinations of Compound 1 at 15 or 50 mpk QD with Compound B at 20 mpk BID demonstrated better anti-tumor activity than either single agent (FIGS. 1C and 1D). All treatment groups had no significant impact on animal body weight throughout the study.

TABLE 5

| | Mean TV | |
|---|---|---|
| | (D 21) | |
| Group | (mm³ ± SEM) | TGI (%) |
| vehicle | 1820.9 ± 125.7 | N/A |
| Compound B, 20 mpk p.o. BID | 799.8 ± 73.2 | 56% |
| Compound 1, 5 mpk p.o. QD | 1293.3 ± 100.0 | 29% |
| Compound 1, 15 mpk p.o. QD | 929.5 ± 73.6 | 49% |
| Compound 1, 50 mpk p.o. QD | 927.5 ± 100.6 | 49% |
| Compound B, 20 mpk combo | 687.2 ± 90.2 | 62% |
| with Compound 1 5 mpk | | |
| Compound B, 20 mpk combo | 475.7 ± 28.8 | 74% |
| with Compound 1 15 mpk | | |
| Compound B, 20 mpk combo | 530.5 ± 37.7 | 71% |
| with Compound 1 50 mpk | | |

### Example 6: Clinical Study

### 1. Method

### Study Design/Objectives

A phase 1 study (dose escalation and safety expansion) to determine safety, tolerability, maximum tolerated dose (MTh), and recommended phase 2 dose (RP2D) of Compound 1 in patients with R/R B-cell malignancies was conducted. (Table 6-1A).

Dose-escalation (Part 1) occurs in independent cohorts categorized by patient disease type. These cohorts are continued until a recommended Phase 2 dose (RP2D) is identified, which is then used in corresponding expansion cohorts (Part 2).

Part 1 Monotherapy Ramp-Up Schedule and Dose Finding
1) Cohort 1A: Cohort 1A consists of patients with relapsed/refractory B-cell non-Hodgkin lymphoma (R/R B-cell NHL) excluding mantle cell lymphoma (MCL). These patients are expected to be at low risk of tumor lysis syndrome (low TLS risk), and are treated with a short ramp-up schedule reaching target dose on Day 3. Patients in this cohort receive escalating doses of Compound 1 monotherapy: 40 mg, 80 mg, 160 mg, 320 mg, and 640 mg (unless adjusted by Safety Monitoring Committee [SMC] recommendation).
2) Cohort 1B is opened, when ≥1 tolerable dose level of Cohort 1A was determined. It consists of patients with low-tumor-burden R/R CLL/SLL. This cohort pursues dose finding, including evaluation of both ramp-up schedule and target dose. Patients increase the ramp-up dose weekly until the target dose for the cohort is reached. The ramp-up steps is 1 mg, 2 mg, 5 mg, 10 mg, 20 mg, 40 mg, 80 mg, 160 mg, 320 mg, and 640 mg (unless adjusted by SMC recommendation). This cohort will not be dosed until the SMC.
3) Cohort 1C: This Cohort consists of patients with high-tumor-burden R/R chronic lymphocytic leukemia/small lymphocytic lymphoma (R/R CLL/SLL). This cohort will not be dosed until the RP2D for Cohort 1B is established. The objective of this cohort is to confirm, in patients with high-tumor-burden CLL/SLL, the safety of the monotherapy ramp-up schedule and RP2D established in patients with low-tumor-burden CLL/SLL.
4) Cohort 1D: This Cohort consists of patients with R/R MCL. This cohort will not be dosed until the RP2D for Cohort 1B is established. The objective of this cohort is to confirm, in patients with R/R MCL, the safety of the monotherapy ramp-up schedule and RP2D established in patients with low tumor-burden CLL/SLL.
Cohort 1D′: This Cohort consists of patients with R/R MCL. And, the target dosage are 160 and 320 mg.
5) Cohort 1E: This Cohort consists of patients with R/R waldenstrom macroglobulinemia (R/R WM). This cohort will not be dosed until the RP2D for Cohort 1B is established. The objective of this cohort is to confirm, in patients with R/R WM, the safety of the monotherapy ramp-up schedule and RP2D established in patients with low tumor-burden CLL/SLL.

All dose cohorts would be reviewed by a safety monitoring committee (SMC) before opening subsequent dose levels or declaring an MTD/RP2D.

Part 2 Monotherapy Expansion Cohorts
1) Cohort 2A: R/R indolent NHL (follicular lymphoma [FL] and marginal zone lymphoma [MZL]).
2) Cohort 2B: R/R aggressive NHL (diffuse large B-cell lymphoma [DLBCL] and transformed B-cell NHL).
3) Cohort 2C: R/R CLL/SLL with low tumor burden.
4) Cohort 2D: R/R CLL/SLL with high tumor burden.
5) Cohort 2E: R/R CLL/SLL with prior venetoclax (yen) treatment.
6) Cohort 2F: R/R MCL.
7) Cohort 2G: R/R WM.

The study also includes dose escalation and expansion cohorts for the combination of Compound 1 and Bruton tyrosine kinase (BTK) inhibitor Zanubrutinib in patients with selected B-cell malignancy, such as CLL/SLL and mantle cell lymphoma (MCL). (Table 6-2A). Patients in the combination therapy cohorts receive Zanubrutinib 320 mg daily (160 mg twice a day [BID] or 320 mg once a day [QD]) beginning 8-12 weeks before Compound 1 is introduced. And, the corresponding dose-escalation and expansion are conducted in Part 3 and Part 4):

Part 3 Combination Ramp-Up Schedule and Dose Finding

Cohort 3A and Cohort 3B study patients with R/R CLL/SLL or R/R MCL, respectively, to establish the RP2D and MTD or MAD for Compound 1 combined with zanubrutinib 320 mg daily.

Dose finding is pursued in Patients with R/R CLL/SLL or R/R MCL who are Bel-2-inhibitor naive and have not progressed on a BTK inhibitor, including evaluation of ramp-up schedule and target dose of Compound 1 when used in combination with Zanubrutinib. The Compound 1 dose vary but the Zanubrutinib dose is fixed at 320 mg/day (160 mg twice daily or 320 mg once daily). The ramp-up dose increases weekly until the target dose for the cohort is reached. The ramp-up steps for Compound 1 are 1 mg, 2 mg, 5 mg, 10 mg, 20 mg, 40 mg, 80 mg, 160 mg, 320 mg, and 640 mg (unless adjusted by SMC recommendation).

Cohort 3A is dosed until the SMC identifies a safe starting dose for this higher-TLS-risk disease (based on data from Part 1) and sets the initial target dose, after the target dose+1 has been cleared as safe in Part 1 cohorts. Patients in Cohort 3B are expected to have a similar TLS risk as compared to CLL/SLL and patients in this cohort may begin dose finding at or below the current highest tolerable dose (or RP2D, if established) for CLL/SLL patients in combination dose-finding (Cohort 3A).

Part 4 Combination Expansion Cohort
1) Cohort 4A studies patients with R/R CLL/SLL with the ramp-up schedule and target dose of Compound 1, identified in Cohort 3A, given with zanubrutinib 320 mg/day (administered 160 mg twice daily or 320 mg once daily) to expand the safety evaluation of the treatment dose and ramp-up schedule.
2) Cohort 4B studies patients with treatment naïve (TN) CLL/SLL and uses the same dose and schedule as for Cohort 4A, unless modified by the SMC.
3) Cohort 4C studies patients with RJR MCL and uses the RP2D declared from Cohort 3B, unless modified by the SMC.

In all Cohorts of the phase 1 study, Compound 1 is orally administrated once daily (QD).

TABLE 6-1A
Study Scheme (Monotherapy Cohorts)

| Part 1: Dose Finding | | | | | Part 2: Expansion | | | |
|---|---|---|---|---|---|---|---|---|
| | | | Planned | | | | | Planned |
| Cohort | Population | Disease | N | RP2D | Cohort | Population | Disease | N |
| 1A | R/R | NHL | 15-30 | RP2D per | 2A | R/R | Indolent NHL | 10 |
| | | (FL, DLBCL, | | disease | | (Food Effect) | (FL, MZL) | |
| | | MZL, or | | type will | | | | |
| | | transformed | | be | | | | |
| | | NHL) | | decided | | | | |
| 1B | R/R | CLL/SLL | 15-30 | based on | 2B | R/R | Aggressive | 10 |
| | (low TLS | | | SMC | | (Food Effect) | NHL | |
| | risk) | | | review of | | | (DLBCL, | |
| | | | | available | | | transformed | |
| | | | | safety and | | | NHL) | |
| 1C | R/R | CLL/SLL | 3-6 | activity | 2C | R/R | CLL/SLL | 20 |
| | (high TLS | | | data. | | (low TLS risk) | | |
| | risk*) | | | | | | | |
| 1D | R/R | MCL | 3-6 | | 2D | R/R | CLL/SLL | 10 |
| | | | | | | (high TLS | | |
| | | | | | | risk*) | | |
| 1E | R/R | WM | 3-6 | | 2E | R/R | CLL/SLL | 10 |
| | | | | | | (prior ven) | | |
| | | | | | 2F | R/R | MCL | 20 |
| | | | | | 2G | R/R | WM | 20 |

TABLE 6-2A
Study Scheme (Combination Cohorts)

| Part 3: Dose Finding | | | | | Part 4: Expansion | | | |
|---|---|---|---|---|---|---|---|---|
| | | | Planned | | | | | Planned |
| Cohort | Population | Disease | N | RP2D | Cohort | Population | Disease | N |
| 3A | R/R | CLL/SLL | 15-30 | RP2D per | 4A | R/R | CLL/SLL | 30 |
| 3B | R/R | MCL | 3-6 | cohort will be | 4B | TN | CLL/SLL | 20 |
| | | | | decided | 4C | R/R | MCL | 20 |
| | | | | based on | | | | |
| | | | | SMC review | | | | |
| | | | | of available | | | | |
| | | | | safety and | | | | |
| | | | | activity data. | | | | |

Data for Cohorts 1A, 1B, 3A, and 3B presented here.
*High TLS risk defined as the presence of any lymph node ≥10 cm or the presence of any lymph node ≥5 cm with concurrent absolute lymphocyte count (ALC)≥25×109/L.

Key Eligibility Criteria

Each patient eligible to participate in this study must meet all of the following criteria.
1. Age 18 years or older
2. Confirmed diagnosis of one of the following:

NHL Cohorts
a. MZL, i.) R/R extranodal, splenic, or nodal MZL defined as disease that relapsed after, or was refractory to, at least 1 prior therapy; ii.) Active disease requiring treatment.
b. FL, i). R/R FL (Grade 1, 2 or 3a based on the WHO 2008 classification of tumors of hematopoietic and lymphoid tissue) and defined as disease that relapsed after, or was refractory to, at least 1 prior systemic therapy; ii.) Active disease requiring treatment.
c. DLBCL, i.) R/R DLBCL (including all subtypes of DLBCL) defined as disease that relapsed after, or was refractory to, at least 1 prior systemic therapy and has either progressed following or is not a candidate for autologous stem cell transplant (due to comorbidities or non-responsiveness to salvage chemotherapy); ii.) Active disease requiring treatment.
d. Transformed indolent B-cell NHL, i.) Any lymphoma otherwise eligible for Part 1 that has transformed into a more aggressive lymphoma. Patients with transformation from CLL or SLL (Richter's transformation) are not eligible for Part 1. ii.) Active disease requiring treatment.

MCL Cohorts
e. WHO-defined MCL, i.) R/R MCL defined as disease that relapsed after, or was refractory to, at least 1 prior systemic therapy; ii.) Requiring treatment in the opinion of the investigator.

CLL/SLL Cohorts:
f. CLL/SLL diagnosis that meets the International Workshop on Chronic Lymphocytic Leukemia criteria (Hallek et al 2008).
i. Meeting the following sets of prior treatment criteria: (1) For R/R cohorts (Cohorts 1C, 2C, 2D, 2E, 3A, and 4A), disease that relapsed after, or was refractory to, at least 1 prior therapy; (2) For the venetoclax-treated cohort (Cohort 2E): prior therapy history must include progression after a therapy containing ≥2 months of venetoclax treatment (monotherapy or combination); (3) For the treatment-naïve cohort (Cohort 4B), patients should have no prior treatment for CLL/SLL (other than 1 aborted regimen <2 weeks in duration and >4 weeks before enrollment).
ii. Requiring treatment

WM Cohorts:
g. WHO-refined WM (clinical and definitive histologic diagnosis), i.) R/R disease defined as disease that relapsed after, or was refractory to, at least 1 prior therapy; ii.) Meeting at least 1 criterion for treatment according to consensus panel criteria from the Seventh International Workshop on Waldenstrom's Macroglobulinemia (Dimopoulos et al 2014).

Measurable Disease by Computed Tomography/Magnetic Resonance Imaging, Defined as:
a. CLL: at least 1 lymph node >1.5 cm in longest diameter and measurable in 2 perpendicular dimensions or clonal lymphocytes on flow cytometry.
b. DLBCL, FL, MZL, MCL, or SLL: at least 1 lymph node >1.5 cm in longest diameter OR 1 extranodal lesion >1.0 cm in the longest diameter, measurable in 2 perpendicular dimensions. For MZL isolated splenomegaly is considered measurable for this study.
c. WM: serum IgM level >0.5 g/Dl.
3. Measurable disease by computed tomography (CT)/magnetic resonance imaging (MRI).
4. Eastern Cooperative Oncology Group (ECOG) performance status of 0 to 2.
6. Adequate pancreatic function indicated by:
   Serum amylase ≤1.5× upper limit of normal (ULN)
   Serum lipase ≤1.5×ULN

Key Exclusion Criteria:
Known central nervous system involvement by lymphoma/leukemia
Known plasma cell neoplasm, prolymphocytic leukemia, history of or currently suspected Richter's syndrome.

### Dose Escalation

For dose escalation, patients were enrolled in 1 of 5 planned daily oral Compound 1 dose levels in cohorts of at least 3 patients: 40 mg, 80 mg, 160 mg, 320 mg, and 640 mg daily.

### Dose Ramp-Up

To protect against potential tumor lysis syndrome (TLS), all patients received a dose ramp-up to the target dose level, and the target doses are 40 mg, 80 mg, 160 mg, 320 mg, and 640 mg daily for both monotherapy and combination therapy (Table 5-1B).
1) Patients with NHLs (excluding MCL) as part of Cohorts 1A, 2A, and 2B receive 2-day ramp-up (day 1, 25% of target dose; day 2, 50% of target dose) before reaching the target daily dose (day 3+, 100%).
2) Patients with CLL/SLL, MCL or WM as part of Cohorts 1B, 1C, 1D, 1E, 2C, 2D, 2E, 2F, 2G, 3A, 3B, 4A, 4B, and 4C receive a weekly ramp-up (beginning with 1 mg daily, doubling the dose weekly until the target dose was reached). The dose steps are 1 mg, 2 mg, 5 mg, 10 mg, 20 mg, 40 mg, 80 mg, 160 mg, 320 mg, and 640 mg.

Other TLS prophylaxis included
1) Hydration: oral or intravenous 1.5-2 L/day from ≥1 day before until ≥1 day following each new dose level;
2) Antihyperuricemics (allopurinol; rasburicase as needed): from ≥2 days before first dose until 1 week after reaching final target dose level; and,
3) Hospitalization for observation: TLS labs and PK are monitored frequently.
   NHL: required during ramp-up for at least the first 3 ramp-up doses; and,
   CLL: required for day 1 of each week for at least the first 3 ramp-up doses.

TABLE 5-1B
Ramp-Up Schemes (Example Target Dose of 80 mg)
Two-day Ramp-Up Schemes for Patients With Diseases at Lower Risk of TLS
(Cohorts 1A, 2A, and 2B)

| | | |
|---|---|---|
| 20 mg QD | 40 mg | 80 mg |
| D1 | QD | QD |
| | D2 | D3+ |

Weekly Ramp-up Scheme for Patients With CLL/SLL, MCL, or WM
(Cohorts 1B, 1C, 1D, 1D′, 1E, 2C, 2D, 2E, 2F, 2G, 3A, 3B, 4A, 4B, and 4C)

| | | | | | | | |
|---|---|---|---|---|---|---|---|
| 1 mg QD | 2 mg QD | 5 mg QD | 10 mg | 20 mg | 40 mg | 80 mg | → |
| W1 | W2 | W3 | QD | QD | QD | QD | |
| | | | W4 | W5 | W6 | W7+ | |

D, day; QD, once daily; W, week.

### Reporting, Etc.

Adverse events (AEs) were reported per CTCAE v5.0 (iwCLL for select hematologic toxicities for CLL patients7)

Terminology Criteria for AEs v5.0 (International Workshop on CLL [iwCLL] for select hematologic toxicities for patients with CLL). Response to treatment was assessed by Lugano classification 12 for patients with NHL and by iwCLL guidelines 13 for patients with CLL.

Dose-limiting toxicities (DLTs) during dose escalation were evaluated up until 21 days at the target dose per patient. A Bayesian logistic regression model is used for target dose finding to model the relationship between the dose levels and the dose-limiting toxicities (DLT) rates in dose-level cohorts

### 2. Results

### Disposition and Baseline

In the monotherapy, 7 patients with R/R NHL were treated in Cohort 1A and 2 patients with R/R CLL were treated in Cohorts 1B. And then, totally 36 patients were further enrolled in Cohorts 1A, 1B, 3A, and 3B (Table 5-2A): 1) in the monotherapy, 19 patients with R/R NHL were treated in Cohort 1A, and 6 and 10 patients with R/R CLL were treated in Cohorts 1B and 1C; and, 2) in the combination therapy, 10 patients with R/R CLL were treated in Cohort 3A, and 1 patient with RJR MCL was treated in Cohort 3B.

TABLE 5-2A
Patient Disposition
Monotherapy N = 25, Median follow-up:
2.6 months (range, 0.1-15.7)

| | |
|---|---|
| Cohorts 1A | Cohorts 1B |
| R/R NHL (FL, DLBCL, tNHL, MZL) | R/R CLL/SLL |
| n = 19 | n = 2 |
| Median follow-up: | Median follow-up: |
| 2.6 months | 4.0 months |
| (range, 0.1-15.7) | (range, 3.3-4.6) |
| On treatment: n = 8; | On treatment: n = 2. |
| off treatment n = 11 | |
| (PD: n = 8, AE: n = 1, Other*: n = 1). | |

Combination Therapy N = 11 Median follow-up:
3.3 months (range, 0.1-6.6)

| | |
|---|---|
| Cohorts 3A | Cohorts 3B |
| R/R CLL/SLL | R/R MCL |
| n = 10 | n = 1 |
| Median follow-up: 3.9 months (range, 0.1-6.6) | Follow-up: 0.5 months |

*Includes “other” or “physician decision.

TABLE 6-2B
Patient and Disease Characteristics

| | | | Combination | |
|---|---|---|---|---|
| | Monotherapy | | Therapy | |
| Characteristic | N = 25 | | N = 11 | |
| Age, median (range), y | 76 | (55-86) | 60 | (41-75) |

ECOG performance status, n (%)

| | | | | |
|---|---|---|---|---|
| 0 | 10 | (40) | 7 | (63.6) |
| 1 | 13 | (52) | 4 | (36.4) |

| | | | |
|---|---|---|---|
| 2 | 2 | (8) | 0 |

Disease types, n (%)

| CLL | 6 | (24) | 10 | (90.9) |
|---|---|---|---|---|
| DLBCL | 12 | (48) | — | |
| FL | 4 | (16) | — | |
| MZL | 3 | (12) | — | |
| MCL | 0 | | 1 | (9.1) |
| No. of prior lines of therapy, | 2 | (1-5) | 1 | (1-2) |
| median (range) | | | | |
| Time from end of most recent | 7.7 | (9-49.7) | 45.5 | (1.6-194.4) |
| systemic therapy to first dose | | | | |
| median (range), months | | | | |

### Safety

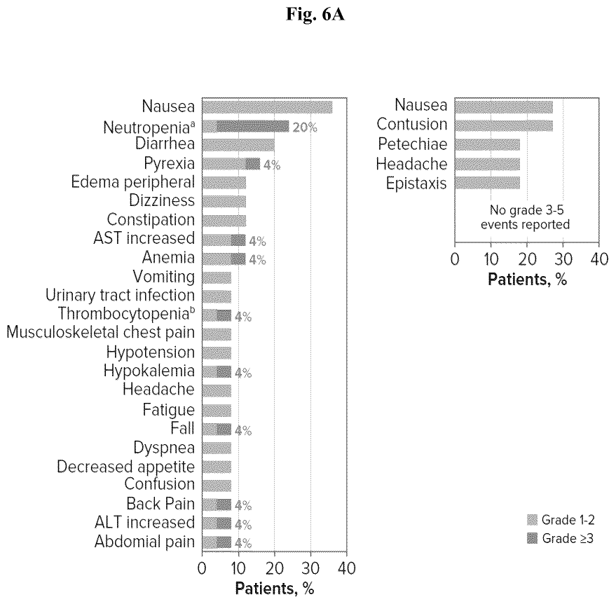

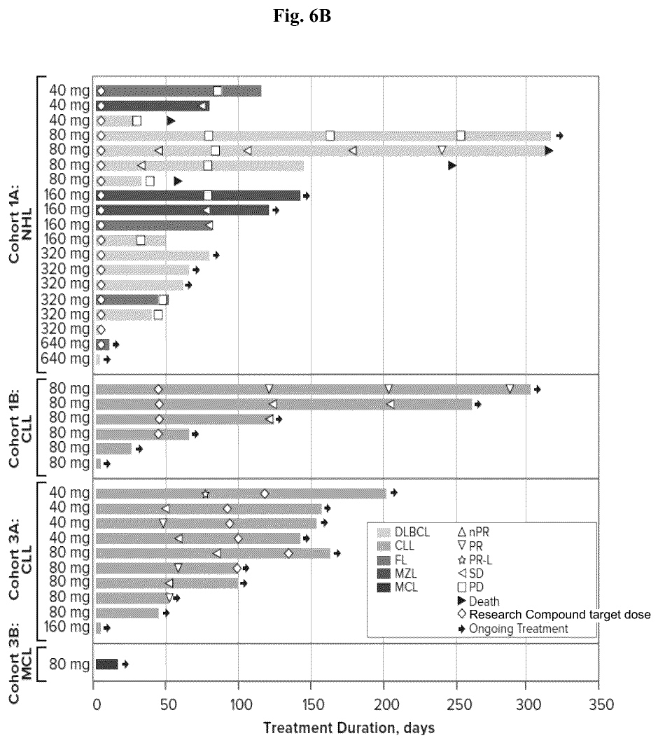

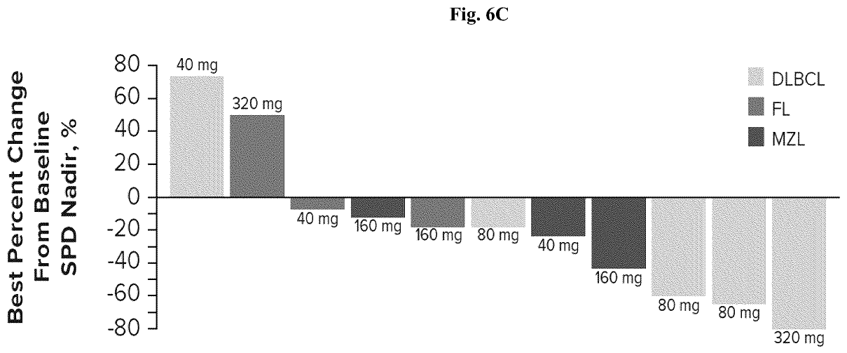

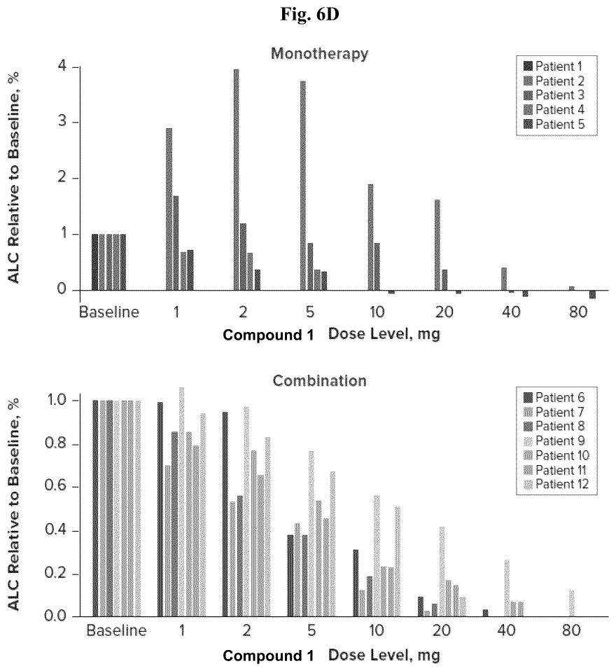

Safety data for 36 patients (monotherapy [N=25], combination therapy [N=11]) received Compound 1 are shown in Table 6-3A and FIG. 6A. Further, safety data for 58 patients received Compound 1 is shown in Table 6-3B. Of the 58 patients receiving monotherapy, 26 with R/R non-Hodgkin lymphoma (NHL; 17 DLBCL, 6 FL, and 3 MZL) received Compound 1≤640 mg and 6 with R/R CLL/SLL received Compound 1≤160 mg. Of the 58 patients receiving combination treatment, 19 with R/R CLL/SLL received Compound 1≤160 mg and 7 with R/R MCL received Compound 1≤80 mg. MTh has not yet been reached. Median follow-up was 3.9 months (range, 0.1-20.4). And, twenty patients of 58 patients discontinued treatment (17 disease progression; 1 AE; 2 other reasons).

In monotherapy, the most common treatment-emergent adverse events (AEs) included nausea. Grade ≥3 AEs reported in patient were observed: abdominal pain, enteritis, small intestinal obstruction, blood alkaline phosphatase increased, GGT increased, platelet count increased, cachexia, pyrexia, back pain, and laboratory TLS. One high-risk patients with CLL on monotherapy had laboratory TLS that resolved with no intervention (laboratory TLS <2%). Two deaths secondary to disease progression were noted.

In combination therapy, 2 grade ≥3 AEs (1 neutropenia, 1 autoimmune hemolytic anemia) were reported.

TABLE 5-3A
Overall Adverse Events (N = 36)

| | | | Combination |
|---|---|---|---|
| | Monotherapy | | Therapy |
| AEs, n (%) | N = 25 | | N = 11 |
| Total | 22 | (88) | 9 (82) |
| Grade ≥3 AEs | 11 | (44) | 0 |
| Serious AEs | 9 | (36) | 0 |
| Leading to death | 2 | (8) ^{a} | 0 |
| AEs leading to hold of Compound 1 | 4 | (16) ^{b} | 0 |
| AEs leading to dose reduction | 0 | | 0 |
| of Compound 1 | | | |
| AEs leading to discontinuation | 1 | (4) ^{c} | 0 |
| of Compound 1 | | | |

^{a} Neither related to study drug; 1 death secondary to disease progression and 1 GI hemorrhage subsequent to bowel surgery.
^{b} ALT increased and GGT increased; neutropenia, pyrexia, and febrile neutropenia; GI hemorrhage and small intestinal obstruction; neutropenia.
^{c} GI hemorrhage subsequent to bowel surgery.

TABLE 6-3B
Overall Adverse Events (N = 58)

| | | |
|---|---|---|
| Any AE in >10% of patients n (%) | Grade ≥3 | All Grade |

Compound 1 Monotherapy (n = 32)

| | | | |
|---|---|---|---|
| Nausea | 0 | 12 | (37.5) |
| Diarrhea | 0 | 8 | (25.0) |
| Fatigue | 0 | 8 | (25.0) |

| | | | | |
|---|---|---|---|---|
| Neutropenia | 6 | (18.8) | 8 | (25.0) |
| Pyrexia | 1 | (3.1) | 6 | (18.8) |

| | | | |
|---|---|---|---|
| Constipation | 0 | 5 | (15.6) |
| Dizziness | 0 | 5 | (15.6) |

| | | | | |
|---|---|---|---|---|
| Fall | 2 | (6.3) | 5 | (15.6) |

| | | | |
|---|---|---|---|
| Headache | 0 | 5 | (15.6) |

| | | | | |
|---|---|---|---|---|
| Abdominal Pain | 2 | (6.3) | 4 | (12.5) |

| | | | |
|---|---|---|---|
| Oedema peripheral | 0 | 4 | (12.5) |

| | | | | |
|---|---|---|---|---|
| Thrombocytopenia | 2 | (6.3) | 4 | (12.5) |

| | | | |
|---|---|---|---|
| Urinary tract infection | 0 | 4 | (12.5) |

Compound 1 + Zanabrutinib Combination (n = 26)

| | | | |
|---|---|---|---|
| Contusion | 0 | 6 | (23.1) |
| Nausea | 0 | 6 | (23.1) |
| Diarrhea | 0 | 5 | (19.2) |
| Fatigue | 0 | 4 | (15.4) |
| Back pain | 0 | 3 | (11.5) |
| Headache | 0 | 3 | (11.5) |
| Petechiae | 0 | 3 | (11.5) |

### Dose Escalation Status

Cohort 1A NHL: 40-mg (n=3; 1 MZL, 2 DLBCL), 80-mg (n=4; 1 FL, 3 DLBCL) and 320-mg (n=3) dose cohorts completed with no disease-limiting toxicities (DLTs); 160-mg (n=3+1) dose cohort showed one DLT of grade 3 febrile neutropenia; and all dose cohorts including 320-mg and 640-mg dose cohort completed, with no MTD reached through 640 mg.

Cohort 1B R/R CLL: started dose escalation at the 80 mg target dose level (n=4) after declared tolerable in Cohort 1A, and one DLT of grade 4 neutropenia was seen. 160 mg, 320 mg and 640 mg dose cohorts are ongoing. Although Cohort 1B only allowed patients with low TLS risk, a patient with high TLS risk was incorrectly enrolled. Retrospective review of baseline CT by site radiologist upgraded the largest node to 6.5×2.4 cm, with absolute lymphocyte count (ALC) 37.4×10⁹/L (n=2, 80 mg target dose level).

Cohort 3A R/R CLL: 40 mg (n=4), 80 mg (n=3) and 160 mg (n=3) dose cohorts completed with no disease-limiting toxicities (DLTs); and, 320-mg and 640-mg dose cohorts are ongoing.

Cohort 3B R/R MCL: 80 mg dose cohorts completed with no disease-limiting toxicities (DLTs); 160 mg, 320-mg and 640-mg dose cohorts are ongoing.

Cohort 4B TN CLL: 160 mg dose cohorts were open, and tolerability and promising activity were seen.

### BCL2 Inhibitor Adverse Events of Interest

TLS: The incorrectly enrolled patient with high baseline TLS risk developed laboratory US and had a major tumor flare on BTK inhibitor withdrawal during early ramp-up, with lactate dehydrogenase 1500, largest node to 5-10 cm, ALC 135.9×109/L. This patient also had baseline and history of hyperuricemia. During dose escalation, patient met criteria for laboratory TLS per Howard criteria8 in late ramp-up at both the 40 mg and 80 mg dose levels. Urate baseline: 430 mmol/L; urate peak: 570 mmol/L; phosphate baseline: 0.35 mmol/L; phosphate peak: 2.16 mmol/L. The patient experienced no sequalae from laboratory TLS and resolved by the next day, and Compound 1 did not need to be held.

In monotherapy, neutropenia was observed in 6 patients (5 experienced grade ≥3 neutropenia), and 2 patients recovered in early Compound 1 treatment.

One patient receiving monotherapy with high baseline TLS risk had a marked tumor flare on BTK inhibitor withdrawal and developed laboratory TLS in late ramp-up. The patient experienced no sequalae from laboratory TLS and resolved by the next day, and Compound 1 did not need to be held.

### Efficacy

Most patients had reduction in sum of product of perpendicular diameters. And, patients with CLL/SLL had notable reductions in absolute lymphocyte count at doses as low as 1 mg. Early efficacy of 36 patients (monotherapy [N=25], combination therapy [N=11]) are shown below.

NHL: no patients with NHL have achieved a response to Compound 1 (FIG. 6B), 2 patients (both 80 mg with DLBCL) have had node reduction and remain on therapy, and, 5 patients have progressed. With continual treatment (about at 5 months of treatment duration), it was observed that 2 patients have achieved a response to Compound 1 including 1 complete response (CR). Decreases in sum of product of perpendicular diameters (SPD) have been seen at all dose levels tested.

CLL/SLL: with monotherapy treatment, 1 of 4 patients with CLL reached first response assessment and achieved partial response at the 80-mg dose level (FIG. 6C) and has del (17p) CLL, wherein 2 responses (partial response or better) were seen with continual treatment. Whereas, with combination treatment, some patients responded with partial response with lymphocytosis or better (n=2 at both 40 mg and 80 mg).

All patients showed significant absolute lymphocyte count (ALC) reductions during dose ramp-up, one patient responded after overcoming initial tumor flare, whereas the other showed reductions even at the 1 mg dose level (FIG. 6D). Significant reduction in absolute ALC was noted among all patients with CLL during ramp-up, with reduction in lymphocytes noted at dose levels as low as 1 mg.

### 3. Conclusion

In the early phase 1 results regarding 9 patients (monotherapy [N=9]) suggest that Compound 1 is tolerable in patients at the dose levels tested. No dose-limiting toxicities (DLTs) were seen across 2 dose levels. Grade ≥3 AEs have been infrequent and manageable, and only 2 patients experienced neutropenia. Risk of TLS appears limited and manageable, and only 1 instance of laboratory TLS was seen in a patient with high TLS-risk. And, preliminary activity in this patient population is being assessed with increased enrollment and follow-up, and enrollment of patients with R/R CLL has only recently started, but decreases in absolute lymphocyte count (ALC) have been seen at the initial ramp-up dose of 1 mg.

Results of 36 patients (monotherapy [N=25], combination therapy [N=11]) suggest that Compound 1 is tolerable in patients, such as those with CLL or NHL at the dose levels tested:
a) only 1 dose-limiting toxicities (DLTs) were seen across 4 dose levels tested in NHL, and 1 DLT was seen in a CLL cohort;
b) grade ≥3 AEs have been infrequent and manageable, and only 2 patients experienced neutropenia;
c) risk of TLS appears limited and manageable, with none seen in combination cohorts; Risk of TLS appears limited and manageable, only 1 instance of laboratory TLS was seen in a patient with CLL who had high TLS-risk;
d) neutropenia has been the most frequent grade ≥3 AE, but has been temporary and not well correlated with treatment dose, and, preliminary activity in this patient population is being assessed with increased enrollment and follow-up Substantial decreases in ALC have been seen during ramp-up for CLL patients, and decreases in absolute lymphocyte count (ALC) have been seen at the initial ramp-up dose of 1 mg; and,
e) evaluation of patients with MCL, treatment-naive CLL, or WM is planned for future cohorts.

According to the result of 58 patients, Compound 1 treatment showed promising efficacy for and an improved safety profile, particularly in combination cohorts. Grade ≥3 neutropenia was uncommon. Compound 1 is tolerable up to doses of 640 mg as monotherapy and up to 160 mg in combination with Zanubrutinib. Dose escalation continues as an MTD has not yet been reached in any dose-escalation cohort. Enrollment continues, with data for Waldenström macroglobulinemia and treatment-naïve CLL/SLL cohorts forthcoming.

In addition, more patients were enrolled in the studies. Totally, 78 patients in the following disposition were dosed, and the corresponding efficacy was estimated.
(1) In monotherapy (N=34), patients with R/R NHL (n=26, median follow-up=6.0 months [range, 1.7-22.0]), R/R CLL/SLL (n=6, median follow-up=8.2 months [range, 5.2-15.0]), and R/R WM (n=2, median follow-up=2.6 months [range, 2.0-3.2]) were treated, wherein the patients with R/R NHL comprised patients FL (n=6), DLBCL (n=17) and MZL (n=3). Among patients with R/R NHL, significant reduction in SPD form baseline were seen in most patients, two of 20 (10%) patients have responded including 1 PR at 160 mg and 1 CR at 320 mg, and 23 patients were off treatment due to progressive disease (n=20), adverse events (n=1) and other or physician decision (n=2). Among patients with R/R WM, one of 2 (50%) have achieved a minor response at 80 mg.
(2) In combination therapy (N=44), patients with R/R CLL/SLL (n=20, median follow-up=5.2 months [range, 0.8-11.8]), R/R MCL (n=10, median follow-up=2.4 months [range, 0.1-5.6]) and TN CLL/SLL (an expansion cohort at 160 mg daily, n=14, median follow-up=2.1 months [range, 0.0-2.8]) were treated. Among patients with R/R MCL, five of 10 (50%) patients have achieved PR or better at either 80 or 100 mg including 1 CR at each dose level, and 1 R/R MCL was off treatment due to progressive disease.
(3) Among patients with CLL/SLL in monotherapy and combination therapy, significant reduction in absolute lymphocyte count (ALC) was noted among all patients with CLL during ramp-up, with reduction in lymphocytes noted at dose levels as low as 1 mg. In the monotherapy, four of 6 (67%) patients have achieved partial response with lymphocytosis (PR-L) or better at either 80 or 160 mg of Compound 1. In the combination therapy, sixteen of 20 (80%) patients with R/R CLL/SLL have achieved PR-L or better across dose levels ranging between 40-320 mg, and 1 patient with R/R CLL/SLL were off treatment due to progressive disease.

The results of 78 patients suggest that Compound 1 is tolerable in patients with CLL or NHL at the dose levels tested. Dose escalation concluded for monotherapy patients with NHL with only 1 DLT seen and no MTD reached, and only 1 DLT was seen amongst monotherapy patients with CLL. Grade ≥3 AEs have been infrequent and manageable.

Findings suggest that the combination of Compound 1 and zanubrutinib is well tolerated, similar to Compound 1 monotherapy. Risk of TLS appears limited and manageable, including laboratory TLS has been seen in only 1 patient with high TLS-risk CLL receiving monotherapy.

In addition, transient neutropenia was the most frequent grade AE, and substantial decreases in ALC have been seen during ramp-up for patients with CLL, with promising early response rates among patients with RJR CLL.

Further, CR and PR cases for patients with CLL were observed when given as monotherapy or combination therapy with Zanubrutinib (Compound B), and significant reduction in SPD has been observed at all dose levels. All doses from 40 mg to 640 mg appeared safe, and the incidence of adverse events did not increase significantly with increasing dose. 40 mg and 80 mg dose are likely less than optimal with less ALC reduction, blood MRD (minimal residual disease) negativity was observed in 160 mg cohorts after 6 months treatment but not in 40 mg and 80 mg cohorts, and 640 mg appears safe but pill burden. Therefore, 320 mg is likely recommended phase 2 dose in CLL, due to it likely gives the best balance between efficacy, safety, and convenience.

The foregoing examples and description of certain embodiments should be taken as illustrating, rather than as limiting the present invention as defined by the claims. As will be readily appreciated, numerous variations and combinations of the features set forth above can be utilized without departing from the present invention as set forth in the claims. All such variations are intended to be included within the scope of the present invention. All references cited are incorporated herein by reference in their entireties.

### REFERENCES

Adams, J. M., and Cory. S. (2007) The Bcl-2 apoptotic switch in cancer development and therapy. Oncogene 26, 1324-1337
Anderson, M. A., Huang, D., and Roberts, A. (2014) Targeting BCL2 for the treatment of lymphoid malignancies. Semin Hematol 51, 219-227
Czabotar, P. E., Lessene, G., Strasser, A., and Adams, J. M. (2014) Control of apoptosis by the BCL-2 protein family: implications for physiology and therapy. Nat Rev Mol Cell Biol 15, 49-63
Egle. A., Harris, A. W., Bath, M. L., O'Reilly, L., and Cory, S. (2004) VavP-Bcl2 transgenic mice develop follicular lymphoma preceded by germinal center hyperplasia. Blood 103, 2276-2283
Kondo, S., Oakes, M. G., and Sorenson, C. M. (2008) Rescue of renal hypoplasia and cystic dysplasia in Bcl-2 −/− mice expressing Bcl-2 in ureteric bud derived epithelia. Dev Dyn 237, 2450-2459
Roberts, A. W. (2016) Targeting apoptotic pathways to treat lymphoid malignancies. Rinsho Ketsueki 57, 2054-2058
Roberts, A. W., and Huang, D. (2017) Targeting BCL2 With BH3 Mimetics: Basic Science and Clinical Application of Venetoclax in Chronic Lymphocyte Leukemia and Related B Cell Malignancies. Clin Pharmacol Ther 101, 89-98
Schenk, R. L., Strasser, A., and Dewson, G. (2017) BCL-2: Long and winding path from discovery to therapeutic target. Biochem Biophys Res Commun 482, 459-469
Tausch, E., Close, W., Dolnik, A., Bloehdorn, J., Chyla, B., Bullinger, L., Dohner, H., Mertens, D., and Stilgenbauer, S. (2019) Venetoclax resistance and acquired BCL2 mutations in chronic lymphocyte leukemia. Haematologica 104, e434-e437.
Veis, D. J., Sorenson, C. M., Shutter, J. R. and Korsmeyer, S. J. (1993) Bcl-2-deficient mice demonstrate fulminant lymphoid apoptosis, polycystic kidneys, and hypopigmented hair. Cell, 75, 229-240.
Yamamura, K., Kamada, S., Ito, S., Nakagawa. K., Ichihashi, M., and Tsujimoto, Y. (1996) Accelerated disappearance of melanocytes in bcl-2-deficient mice. Cancer Res 56, 3546-3550.
Delbridge, A. R., et al., Thirty years of BCL-2: translating cell death discoveries into novel cancer therapies. Nat Rev Cancer, 2016. 16(2): p. 99-109.
Tsujimoto, Y. and C. M. Croce, Analysis of the structure, transcripts, and protein products of bcl-2, the gene involved in human follicular lymphoma. Proc Natl Acad Sci USA, 1986. 83(14): p. 5214-8.
Placzek, W. J., et al., A survey of the anti-apoptotic Bcl-2 subfamily expression in cancer types provides a platform to predict the efficacy of Bcl-2 antagonists in cancer therapy. Cell Death Dis, 2010. 1: p. e40.
Roberts, A. W., et al., Targeting BCL2 with Venetoclax in Relapsed Chronic Lymphocytic Leukemia. N Engl J Med, 2016. 374(4): p. 311-22.
DiNardo, C. D., et al., Venetoclax combined with decitabine or azacitidine in treatment-naive, elderly patients with acute myeloid leukemia. Blood, 2019. 133(1): p. 7-17.
Seymour, J. F., et al., Venetoclax plus rituximab in relapsed or refractory chronic lymphocytic leukaemia: a phase 1b study. Lancet Oncol, 2017. 18(2): p. 230-240.
Jain, N., V. Gandhi, and W. Wierda, Ibrutinib and Venetoclax for First-Line Treatment of CLL. Reply. N Engl J Med, 2019. 381(8): p. 789.
Hillmen, P., et al., Ibrutinib Plus Venetoclax in Relapsed/Refractory Chronic Lymphocytic Leukemia: The CLARITY Study. J Clin Oncol, 2019. 37(30): p. 2722-2729.

What is claimed is:

1. A method of treating B-cell malignancy, the method comprising administering to a subject in need thereof a therapeutically effective amount of a Bcl-2 inhibitor, wherein the Bcl-2 inhibitor is selected from the group consisting of:
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((4-(((4-fluorotetrahydro-2H-pyran-4-yl)methyl)amino)-3-nitrophenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-ethylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-ethylphenyl)pyrrolidin-1-yl)-7-azaspiro[3.5]nonan-7-yl)-N-((4-(((4-fluorotetrahydro-2H-pyran-4-yl)methyl)amino)-3-nitrophenyl)sulfonyl)benzamide;
2-((1H-pyrrolo[2,3-b]pyridin-5-yl)oxy)-4-(2-((S)-2-(2-ethylphenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
(R)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((4-(((4-fluorotetrahydro-2H-pyran-4-yl)methyl)amino)-3-nitrophenyl)sulfonyl)benzamide;
2-((1H-pyrrolo[2,3-b]pyridin-5-yl)oxy)-4-(2-((R)-2-(2-cyclopropylphenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
(S)-2-((1H-pyrrolo[2,3-b]pyridin-5-yl)oxy)-4-(7-(2-(2-cyclopropylphenyl)pyrrolidin-1-v1)-2-azaspiro[3.5]nonan-2-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(7-(2-(2-cyclopropylphenyl)pyrrolidin-1-yl)-2-azaspiro[3.5]nonan-2-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(9-(2-(2-cyclopropylphenyl)pyrrolidin-1-yl)-3-azaspiro[5.5]undecan-3-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(9-(2-(2-cyclopropylphenyl)pyrrolidin-1-yl)-3-azaspiro[5.5]undecan-3-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(6-(2-(2-cyclopropylphenyl)pyrrolidin-1-yl)-2-azaspiro[3.3]heptan-2-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(6-(2-(2-cyclopropylphenyl)pyrrolidin-1-yl)-2-azaspiro[3.3]heptan-2-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(3-chloro-2-(dimethylamino)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)benzamide;
N-((4-((((R)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)benzamide;
(S)2-((1H-pyrrolo[2,3-b]pyridin-5-yl)oxy)-4-(2-((S)-2-(2-cyclopropylphenyl)pyrrolidin-1-yl)-8-azaspiro[4.5]decan-8-yl)-N-((3-nitro-4-(((tetrahydro-2H-pyran-4-yl)methyl)amino)phenyl)sulfonyl)benzamide;
(R) 2-((1H-pyrrolo[2,3-b]pyridin-5-yl)oxy)-1-(2-((S)-2-(2-cyclopropylphenyl)pyrrolidin-1-yl)-8-azaspiro[4.5]decan-8-yl)-N-((3-nitro-4-(((tetrahydro-2H-pyran-4-yl)methyl)amino)phenyl)sulfonyl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-ethylphenyl)pyrrolidin-1-yl)-7-azaspiro[3.5]nonan-7-yl)benzamide;
N-((4-((((R)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-ethylphenyl)pyrrolidin-1-yl)-7-azaspiro[3.5]nonan-7-yl)benzamide;
(S)-2-((1H-pyrrolo[2,3-b]pyridin-5-yl)oxy)-4-(8-(2-(2-cyclopropylphenyl)pyrrolidin-1-yl)-2-azaspiro[4.5]decan-2-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-isopropylphenyl)pyrrolidin-1-yl)-7-azaspiro[3.5]nonan-7-yl)benzamide;
(S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-N-((4-(((4-fluorotetrahydro-2H-pyran-4-yl)methyl)amino)-3-nitrophenyl)sulfonyl)-4-(2-(2-(2-isopropylphenyl)pyrrolidin-1-yl)-7-azaspiro[3.5]nonan-7-yl)benzamide;
N-((4-((((R)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-N-((4-((((1s,4s)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((R)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-N-((4-((((1s,4s)-4-hydroxy-4-methylcyclohexyl)methyl)amino)-3-nitrophenyl)sulfonyl)-4-(2-((R)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-4-(2-(2-(2-cyclobutylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-isobutylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(3-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-N-((3-nitro-4-(((tetrahydro-2H-pyran-4-yl)methyl)amino)phenyl)sulfonyl)-4-(2-(2-(o-tolyl)pyrrolidin-1-yl)-7-azaspiro[3.5]nonan-7-yl)benzamide;
(S)-2-((1H-pyrrolo[2,3-b]pyridin-5-yl)oxy)-4-(2-(2-(2-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-bromophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(2-(2-(2-chlorophenyl)pyrrolidin-1-yl)-7-azaspiro[3.5]nonan-7-yl)-N-((3-nitro-4-(((tetrahydro-2H-pyran-1-yl)methyl)amino)phenyl)sulfonyl)benzamide;
(S)-2-((1H-pyrrolo[2,3-b]pyridin-5-yl)oxy)-4-(2-(2-(3-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(2-(2-(3-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(4-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-ethoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-(dimethylamino)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-((S)-2-(2-(dimethylamino)phenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-(2-((S)-2-(2-(bis(methyl-d3)amino)phenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-N-((3-nitro-4-(((tetrahydro-2H-pyran-4-yl)methyl)amino)phenyl)sulfonyl)-4-(2-(2-(2-(pyrrolidin-1-yl)phenyl)pyrrolidin-1-yl)-7-azaspiro[3.5]nonan-7-yl)benzamide;
(S)-2-((1H-pyrrolo[2,3-b]pyridin-5-yl)oxy)-4-(2-(2-(2-(1-methyl-1,2,3,6-tetrahydropyridin-4-yl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-(1-methylpiperidin-4-yl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-methoxyphenyl)pyrrolidin-1-yl)-7-azaspiro[3.5]nonan-7-yl)-N-((3-nitro-1-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-(methoxymethyl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-(hydroxymethyl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(2-(2-(3-chloro-2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(3-chloro-2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(5-chloro-2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(2-(2-(3-chloro-2-ethylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S or R)-2-((1H-pyrrolo[2,3-b]pyridin-5-yl)oxy)-4-(2-(2-(3-chloro-2-ethylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2,4-dicyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2,5-dicyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(3-(2-chlorophenyl)thiophen-2-yl)pyrrolidin-1-yl)-7-azaspiro[3.5]nonan-7-yl)-N-((3-nitro-4-(((tetrahydro-2H-pyran-1-yl)methyl)amino)phenyl)sulfonyl)benzamide;
2-((I H-pyrrolo[2,3-b]pyridin-5-yl)oxy)-4-(2-(2-(2-cyclopropylphenyl)-4-methylpyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(4-cyclopropyl-2-(2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4-phenyl-2,5-dihydro-1H-pyrrol-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4,4-dimethylpyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4,4-difluoropyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4-(trifluoromethyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-(2-(2-(2-cyclopropylphenyl)-4-(dimethylamino)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4-(2-(dimethylamino)ethoxy)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-3,3-dimethylpyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-(((((1s,4s) or (1r,4r))-4-((dimethyl(oxo)-l6-sulfaneylidene)amino)cyclohexyl)methyl)amino)-3-nitrophenyl)sulfonyl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-(methyl(3-nitro-4-(((tetrahydro-2H-pyran-4-yl)methyl)amino)phenyl)(oxo)-l6-sulfaneylidene)benzamide;
2-((1H-pyrrolo[2,3-b]pyridin-5-yl)oxy)-N-((4-(((-3-oxabicyclo[3.1.0]hexan-6-yl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((4-(((4-hydroxy-4-(trifluoromethyl)cyclohexyl)methyl)amino)-3-nitrophenyl)sulfonyl)benzamide;
2-((1H-pyrrolo[2,3-b]pyridin-5-yl)oxy)-N-((4-((((1r,4r)-4-hydroxy-4-(trifluoromethyl)cyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-N-((4-((((1s,4s)-4-hydroxy-4-(trifluoromethyl)cyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-4-(2-((S)-2-(2-isopropylphenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)benzamide;
2-((1H-pyrrolo[2,3-b]pyridin-5-yl)oxy)-4-(2-((S)-2-(2-isopropylphenyl)pyrrolidin-1-yl)-7-azaspiro[3.5]nonan-7-yl)-N-((4-((((S)-4-methylcyclohex-3-en-1-yl)methyl)amino)-3-nitrophenyl)sulfonyl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-(prop-1-en-2-yl)phenyl)pyrrolidin-1-yl)-7-azaspiro[3.5]nonan-7-yl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-propylphenyl)pyrrolidin-1-yl)-7-azaspiro[3.5]nonan-7-yl)benzamide;
2-((1H-pyrrolo[2,3-b]pyridin-5-yl)oxy)-4-(6-((S)-2-(2-cyclopropylphenyl)pyrrolidin-1-yl)-2-azaspiro[3.3]heptan-2-yl)-N-((4-((((1r,4r)-4-hydroxy-4-methylcyclohexyl)methyl)amino)-3-nitrophenyl)sulfonyl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(6-((S)-2-(2-cyclopropylphenyl)pyrrolidin-1-yl)-2-azaspiro[3.3]heptan-2-yl)benzamide;
N-((4-((((R)-1,4-dioxan-2-yl)methyl)amino)-3-nitrophenyl)sulfonyl)-2-((1H-pyrrolo[2,3-b]pyridin-5-yl)oxy)-4-(6-((S)-2-(2-cyclopropylphenyl)pyrrolidin-1-yl)-2-azaspiro[3.3]heptan-2-yl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(6-((S)-2-(2-isopropylphenyl)pyrrolidin-1-yl)-2-azaspiro[3.3]heptan-2-yl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(6-((S)-2-(2-isopropylphenyl)pyrrolidin-1-yl)-2-azaspiro[3.3]heptan-2-yl)benzamide;
2-((1H-pyrrolo[2,3-b]pyridin-5-yl)oxy)-4-(6-((S)-2-(2-ethylphenyl)pyrrolidin-1-yl)-2-azaspiro[3.3]heptan-2-yl)-N-((4-((((1r,4r)-4-hydroxy-4-methylcyclohexyl)methyl)amino)-3-nitrophenyl)sulfonyl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(6-((S)-2-(2-ethylphenyl)pyrrolidin-1-yl)-2-azaspiro[3.3]heptan-2-yl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((3-nitro-4-((2-(tetrahydro-2H-pyran-4-yl)ethyl)amino)phenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((4-((2-morpholinoethyl)amino)-3-nitrophenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((3-nitro-4-((2-(3-oxomorpholino)ethyl)amino)phenyl)sulfonyl)benzamide;
2-((1H-pyrrolo[2,3-b]pyridin-5-yl)oxy)-N-((4-(((3-oxabicyclo[3.1.0]hexan-6-yl)methyl)amino)-3-nitrophenyl)sulfonyl)-4-(2-((S)-2-(2-cyclopropylphenyl)pyrrolidin-1-yl)-7-azaspiro(3.5 (nonan-7-yl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-(((2,6-dimethyltetrahydro-2H-pyran-4-yl)methyl)amino)-3-nitrophenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((3-nitro-4-(((2,2,6,6-tetramethyltetrahydro-2H-pyran-4-yl)methyl)amino)phenyl)sulfonyl)benzamide;
2-((1H-pyrrolo[2,3-b]pyridin-5-yl)oxy)-4-(2-((S)-2-(2-cyclopropylphenyl)pyrrolidin-1-yl)-6-azaspiro[3.4]octan-6-yl)-N-((4-((((1r,4r)-4-hydroxy-1-methylcyclohexyl)methyl)amino)-3-nitrophenyl)sulfonyl)benzamide;
2-((1H-pyrrolo[2,3-b]pyridin-5-yl)oxy)-4-(6-((S)-2-(2-cyclopropylphenyl)pyrrolidin-1-yl)-2-azaspiro[3.4]octan-2-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-((7R or 7S)-7-((S)-2-(2-cyclopropylphenyl)pyrrolidin-1-yl)-2-azaspiro[4.4]nonan-2-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-((7S or 7R)-7-((S)-2-(2-cyclopropylphenyl)pyrrolidin-1-yl)-2-azaspiro[4.4]nonan-2-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-(((2,2-dimethyltetrahydro-2H-pyran-4-yl)methyl)amino)-3-nitrophenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((4-(3-methyl-3-((tetrahydro-2H-pyran-4-yl)methyl)ureido)-3-nitrophenyl)sulfonyl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-phenylpyrrolidin-1-yl)-7-azaspiro[3.5]nonan-7-yl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-((((cis or trans)-4-hydroxytetrahydrofuran-2-yl)methyl)amino)-3-nitrophenyl)sulfonyl)benzamide; and
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-((((trans or cis)-4-hydroxytetrahydrofuran-2-yl)methyl)amino)-3-nitrophenyl)sulfonyl)benzamide;
or a pharmaceutically acceptable salt thereof, or a stereoisomer thereof.

2. A method of treating B-cell malignancies in a subject, the method comprising administering to the subject in need thereof a therapeutically effective amount of a Bcl-2 inhibitor, in combination with a therapeutically effective amount of (S)-7-(1-acryloylpiperidin-4-yl)-2-(4-phenoxyphenyl)-4,5,6,7-tetra-hydropyrazolo[1,5-a]pyrimidine-3-carboxamide (Compound B) or a pharmaceutically acceptable salt thereof, wherein the Bcl-2 inhibitor is selected from the group consisting of:
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((4-(((4-fluorotetrahydro-2H-pyran-4-yl)methyl)amino)-3-nitrophenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-ethylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-ethylphenyl)pyrrolidin-1-yl)-7-azaspiro[3.5]nonan-7-yl)-N-((4-(((4-fluorotetrahydro-2H-pyran-4-yl)methyl)amino)-3-nitrophenyl)sulfonyl)benzamide;
2-((1H-pyrrolo[2,3-b]pyridin-5-yl)oxy)-4-(2-((S)-2-(2-ethylphenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
(R)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((4-(((4-fluorotetrahydro-2H-pyran-4-yl)methyl)amino)-3-nitrophenyl)sulfonyl)benzamide;
2-((1H-pyrrolo[2,3-b]pyridin-5-yl)oxy)-4-(2-((R)-2-(2-cyclopropylphenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
(S)-2-((1H-pyrrolo[2,3-b]pyridin-5-yl)oxy)-4-(7-(2-(2-cyclopropylphenyl)pyrrolidin-1-yl)-2-azaspiro[3.5]nonan-2-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(7-(2-(2-cyclopropylphenyl)pyrrolidin-1-yl)-2-azaspiro[3.5]nonan-2-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(9-(2-(2-cyclopropylphenyl)pyrrolidin-1-yl)-3-azaspiro[5.5]undecan-3-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(9-(2-(2-cyclopropylphenyl)pyrrolidin-1-yl)-3-azaspiro[5.5]undecan-3-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(6-(2-(2-cyclopropylphenyl)pyrrolidin-1-yl)-2-azaspiro[3.3]heptan-2-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(6-(2-(2-cyclopropylphenyl)pyrrolidin-1-yl)-2-azaspiro[3.3]heptan-2-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(3-chloro-2-(dimethylamino)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)benzamide;
N-((4-((((R)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)benzamide;
(S)2-((1H-pyrrolo[2,3-b]pyridin-5-yl)oxy)-4-(2-(S)-2-(2-cyclopropylphenyl)pyrrolidin-1-yl)-8-azaspiro[4.5]decan-8-yl)-N-((3-nitro-4-(((tetrahydro-2H-pyran-4-yl)methyl)amino)phenyl)sulfonyl)benzamide;
(R) 2-((1H-pyrrolo[2,3-b]pyridin-5-yl)oxy)-4-(2-((S)-2-(2-cyclopropylphenyl)pyrrolidin-1-yl)-8-azaspiro[4.5]decan-8-yl)-N-((3-nitro-4-(((tetrahydro-2H-pyran-4-yl)methyl)amino)phenyl)sulfonyl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-ethylphenyl)pyrrolidin-1-yl)-7-azaspiro[3.5]nonan-7-yl)benzamide;
N-((4-((((R)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-ethylphenyl)pyrrolidin-1-yl)-7-azaspiro[3.5]nonan-7-yl)benzamide;
(S)-2-((1H-pyrrolo[2,3-b]pyridin-5-yl)oxy)-4-(8-(2-(2-cyclopropylphenyl)pyrrolidin-1-yl)-2-azaspiro[4.5]decan-2-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-isopropylphenyl)pyrrolidin-1-yl)-7-azaspiro[3.5]nonan-7-yl)benzamide;
(S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-N-((4-(((4-fluorotetrahydro-2H-pyran-4-yl)methyl)amino)-3-nitrophenyl)sulfonyl)-4-(2-(2-(2-isopropylphenyl)pyrrolidin-1-yl)-7-azaspiro[3.5]nonan-7-yl)benzamide;
N-((4-((((R)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-isopropylphenyl)pyrrolidin-1-v1)-7-azaspiro[3.5]nonan-7-yl)benzamide;
2-((1H-pyrrolo[2,3-b]pyridin-5-yl)oxy)-N-((4-((((1s,4s)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-N-((4-((((1r,4r)-4-hydroxy-4-methyl cyclohexyl)methyl)amino)-3-nitrophenyl)sulfonyl)-4-(2-((R)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-N-((4-((((1s,4s)-4-hydroxy-4-methylcyclohexyl)methyl)amino)-3-nitrophenyl)sulfonyl)-4-(2-((R)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-4-(2-(2-(2-cyclobutylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-isobutylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(3-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-N-((3-nitro-4-(((tetrahydro-2H-pyran-4-yl)methyl)amino)phenyl)sulfonyl)-4-(2-(2-(o-tolyl)pyrrolidin-1-yl)-7-azaspiro[3.5]nonan-7-yl)benzamide;
(S)-2-((1H-pyrrolo[2,3-b]pyridin-5-yl)oxy)-4-(2-(2-(2-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-bromophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(2-(2-(2-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(3-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(2-(2-(3-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(4-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-ethoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-(dimethylamino)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-((S)-2-(2-(dimethylamino)phenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-(2-((S)-2-(2-(bis(methyl-d3)amino)phenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-N-((3-nitro-4-(((tetrahydro-2H-pyran-4-yl)methyl)amino)phenyl)sulfonyl)-4-(2-(2-(2-(pyrrolidin-1-yl)phenyl)pyrrolidin-1-yl)-7-azaspiro[3.5]nonan-7-yl)benzamide;
(S)-2-((1H-pyrrolo[2,3-b]pyridin-5-yl)oxy)-4-(2-(2-(2-(1-methyl-1,2,3,6-tetrahydropyridin-4-yl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-(1-methylpiperidin-4-yl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-methoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-(methoxymethyl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-(hydroxymethyl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(2-(2-(3-chloro-2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(3-chloro-2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(5-chloro-2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(2-(2-(3-chloro-2-ethylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S or R)-2-((1H-pyrrolo[2,3-b]pyridin-5-yl)oxy)-4-(2-(2-(3-chloro-2-ethylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2,4-dicyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2,5-dicyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(3-(2-chlorophenyl)thiophen-2-yl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4-methylpyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(4-cyclopropyl-2-(2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4-phenyl-2,5-dihydro-1H-pyrrol-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4,4-dimethylpyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4,4-difluoropyrrolidin-1-yl)-7-azaspiro[3.5]nonan-7-yl)-N-((3-nitro-4-((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4-(trifluoromethyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-(2-(2-(2-cyclopropylphenyl)-4-(dimethylamino)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4-(2-(dimethylamino)ethoxy)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-3,3-dimethylpyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-(((((1s,4s) or (1r,4r))-4-((dimethyl(oxo)-l6-sulfaneylidene)amino)cyclohexyl)methyl)amino)-3-nitrophenyl)sulfonyl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-(methyl(3-nitro-4-(((tetrahydro-2H-pyran-4-yl)methyl)amino)phenyl)(oxo)-l6-sulfaneylidene)benzamide;
2-((1H-pyrrolo[2,3-b]pyridin-5-yl)oxy)-N-((4-(((-3-oxabicyclo[3.1.0]hexan-6-yl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((4-(((4-hydroxy-4-(trifluoromethyl)cyclohexyl)methyl)amino)-3-nitrophenyl)sulfonyl)benzamide;
2-((1H-pyrrolo[2,3-b]pyridin-5-yl)oxy)-N-((4-((((1r,4r)-4-hydroxy-4-(trifluoromethyl)cyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-N-((4-((((1s,4s)-1-hydroxy-4-(trifluoromethyl)cyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-4-(2-((S)-2-(2-isopropylphenyl)pyrrolidin-1-yl)-7-azaspiro[3.5]nonan-7-yl)-N-((4-((((1r,4r)-1-methoxy-4-methylcyclohexyl)methyl)amino)-3-nitrophenyl)sulfonyl)benzamide;
2-((1H-pyrrolo[2,3-b]pyridin-5-yl)oxy)-4-(2-((S)-2-(2-isopropylphenyl)pyrrolidin-1-yl)-7-azaspiro[3.5]nonan-7-yl)-N-((4-((((S)-4-methylcyclohex-3-en-1-yl)methyl)amino)-3-nitrophenyl)sulfonyl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-(prop-1-en-2-yl)phenyl)pyrrolidin-1-yl)-7-azaspiro[3.5]nonan-7-yl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-propylphenyl)pyrrolidin-1-yl)-7-azaspiro[3.5]nonan-7-yl)benzamide;
2-((1H-pyrrolo[2,3-b]pyridin-5-yl)oxy)-4-(6-((S)-2-(2-cyclopropylphenyl)pyrrolidin-1-yl)-2-azaspiro[3.3]heptan-2-yl)-N-((4-((((1r,4r)-4-hydroxy-4-methylcyclohexyl)methyl)amino)-3-nitrophenyl)sulfonyl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(6-((S)-2-(2-cyclopropylphenyl)pyrrolidin-1-yl)-2-azaspiro[3.3]heptan-2-yl)benzamide;
N-((4-((((R)-1,4-dioxan-2-yl)methyl)amino)-3-nitrophenyl)sulfonyl)-2-((1H-pyrrolo[2,3-b]pyridin-5-yl)oxy)-4-(6-((S)-2-(2-cyclopropylphenyl)pyrrolidin-1-yl)-2-azaspiro[3.3]heptan-2-yl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(6-((S)-2-(2-isopropylphenyl)pyrrolidin-1-yl)-2-azaspiro[3.3]heptan-2-yl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(6-((S)-2-(2-isopropylphenyl)pyrrolidin-1-yl)-2-azaspiro[3.3]heptan-2-yl)benzamide;
2-((1H-pyrrolo[2,3-b]pyridin-5-yl)oxy)-4-(6-((S)-2-(2-ethylphenyl)pyrrolidin-1-yl)-2-azaspiro[3.3]heptan-2-yl)-N-((4-((((1r,4r)-4-hydroxy-4-methylcyclohexyl)methyl)amino)-3-nitrophenyl)sulfonyl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(6-((S)-2-(2-ethylphenyl)pyrrolidin-1-yl)-2-azaspiro[3.3]heptan-2-yl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((3-nitro-4-((2-(tetrahydro-2H-pyran-4-yl)ethyl)amino)phenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((4-((2-morpholinoethyl)amino)-3-nitrophenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((3-nitro-4-((2-(3-oxomorpholino)ethyl)amino)phenyl)sulfonyl)benzamide;
2-((1H-pyrrolo[2,3-b]pyridin-5-yl)oxy)-N-((4-(((3-oxabicyclo[3.1.0]hexan-6-yl)methyl)amino)-3-nitrophenyl)sulfonyl)-4-(2-((S)-2-(2-cyclopropylphenyl)pyrrolidin-1-yl)-7-azaspiro[3.5]nonan-7-yl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-(((2,6-dimethyltetrahydro-2H-pyran-4-yl)methyl)amino)-3-nitrophenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((3-nitro-4-(((2,2,6,6-tetramethyltetrahydro-2H-pyran-4-yl)methyl)amino)phenyl)sulfonyl)benzamide;
2-((1H-pyrrolo[2,3-b]pyridin-5-yl)oxy)-4-(2-((S)-2-(2-cyclopropylphenyl)pyrrolidin-1-yl)-6-azaspiro[3.4]octan-6-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-(6-((S)-2-(2-cyclopropylphenyl)pyrrolidin-1-yl)-2-azaspiro[3.4]octan-2-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-((7R or 7S)-7-((S)-2-(2-cyclopropylphenyl)pyrrolidin-1-yl)-2-azaspiro[4.4]nonan-2-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-((7S or 7R)-7-((S)-2-(2-cyclopropylphenyl)pyrrolidin-1-yl)-2-azaspiro[4.4]nonan-2-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-(((2,2-dimethyltetrahydro-2H-pyran-4-yl)methyl)amino)-3-nitrophenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((4-(3-methyl-3-((tetrahydro-2H-pyran-4-yl)methyl)ureido)-3-nitrophenyl)sulfonyl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-phenylpyrrolidin-1-yl)-7-azaspiro[3.5]nonan-7-yl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-((((cis or trans)-4-hydroxytetrahydrofuran-2-yl)methyl)amino)-3-nitrophenyl)sulfonyl)benzamide; and
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-((((trans or cis)-4-hydroxytetrahydrofuran-2-yl)methyl)amino)-3-nitrophenyl)sulfonyl)benzamide;
or a pharmaceutically acceptable salt thereof, or a stereoisomer thereof.

3. The mothed of claim 1 or 2, wherein the Bcl-2 inhibitor is 2-((1H-pyrrolo[2,3-b]pyridin-5-yl)oxy)-N-((4-((((1r,4r)-1-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide or a pharmaceutically acceptable salt thereof.

4. The method of claim 1 or 2, wherein the B-cell malignancy is relapsed/refractory.

5. The method of claim 1 or 2, wherein the Bcl-2 inhibitor is orally administrated.

6. The method of claim 1 or 2, wherein the Bcl-2 inhibitor is orally administrated at a dose of 1 mg QD to 640 mg QD, according to a dose ramp-up schedule.

7. The method of claim 6, wherein the Bcl-2 inhibitor is orally administrated at a dose according to a daily ramp-up schedule.

8. The method of claim 7, wherein the Bcl-2 inhibitor is orally administrated at a dose according to a daily ramp-up schedule comprising the first dose at day 1, the second dose at day 2, and a recommended dose at day 3 and beyond, wherein the second dose at day 3 and beyond is higher than the second dose at day 2, and the second dose at day 2 is higher than the first dose at day 1.

9. The method of claim 8, wherein the Bcl-2 inhibitor is orally administrated at a dose according to a daily ramp-up schedule comprising the first dose at day 1 at 25% of the recommended dose, the second dose at day 2 at 50% of the recommended dose, and the daily dose at day 3 and beyond at 100% of the recommended dose, wherein the recommended dose is 40 mg, 80 mg, 160 mg, 320 mg, or 640 mg daily.

10. The method of claim 8, wherein the first dose at day 1 is about 10-160 mg/day, the second dose at day 2 is about 20-320 mg/day, and the daily dose at day 3 and beyond is about 40-640 mg/day.

11. The method of claim 8, wherein the first dose at day 1 is about 10, 20, 40, 80 or 160 mg/day, the second dose at day 2 is about 20, 40, 80, 160 or 320 mg/day, and the daily dose at day 3 and beyond is about 40 mg, 80 mg, 160 mg, 320 mg, or 640 mg daily.

12. The method of claim 8, wherein the first dose at day 1 is about 20 mg/day, the second dose at day 2 is about 40 mg/day, and the daily dose at day 3 and beyond is about 80 mg daily.

13. The method of claim 8, wherein the first dose at day 1 is about 40 mg/day, the second dose at day 2 is about 80 mg/day, and the daily dose at day 3 and beyond is about 160 mg daily.

14. The method of claim 8, wherein the first dose at day 1 is about 80 mg/day, the second dose at day 2 is about 160 mg/day, and the daily dose at day 3 and beyond is about 320 mg daily.

15. The method of claim 8, wherein the first dose at day 1 is about 160 mg/day, the second dose at day 2 is about 320 mg/day, and the daily dose at day 3 and beyond is about 640 mg daily.

16. The method of claim 1, wherein the B-cell malignancy is at lower risk of TLS.

17. The method of claim 1, wherein the B-cell malignancy is non-Hodgkin lymphoma (NHL) (excluding MCL) or acute lymphoblastic leukemia (ALL), preferably FL, DLBCL, MZL or transformed NHL.

18. The method of claim 6, wherein the Bcl-2 inhibitor is orally administrated at a dose according to a weekly ramp-up schedule.

19. The method of claim 18, wherein the Bcl-2 inhibitor is orally administrated at a dose according to a weekly ramp-up schedule comprising the first dose at week 1, the second dose at week 2, the third dose at week 3, the fourth dose at week 4, the fifth dose at week 5, a subsequently weekly ramp-up schedule, and a recommended dose at a certain week and beyond, wherein the dose at the subsequent week is at least double of the dose the previous week until the weekly recommended dose has been met, and the subsequently weekly ramp-up schedule is a weekly ramp-up dosing schedule for 0, 1, 2, 3, or 4 weeks.

20. The method of claim 19, wherein the Bcl-2 inhibitor is orally administrated at a dose according to a weekly ramp-up schedule beginning with 1 mg daily at week 1, wherein the dose at the subsequent week is at least double of the dose the previous week until the weekly recommended dose has been met, and the recommended dose is 40 mg, 80 mg, 160 mg, 320 mg, or 640 mg daily.

21. The method of claim 19, wherein the Bcl-2 inhibitor is orally administrated by weekly ramp-up schedule comprising a dose steps of 1 mg, 2 mg, 5 mg, 10 mg, 20 mg, 40 mg, 80 mg, 160 mg, 320 mg or 640 mg daily.

22. The method of claim 19, wherein the first dose at week 1 is about 1 mg/day, the second dose at week 2 is about 2 mg/day, the third dose at week 3 is about 5 mg/day, the fourth dose at week 4 is about 10 mg/day, the fifth dose at week 5 is about 20 mg/day, the sixth dose at week 6 is about 40 mg/day, and the seventh dose at week 7 and beyond is about 80 mg/day.

23. The method of claim 19, wherein the first dose at week 1 is about 1 mg/day, the second dose at week 2 is about 2 mg/day, the third dose at week 3 is about 5 mg/day, the fourth dose at week 4 is about 10 mg/day, the fifth dose at week 5 is about 20 mg/day, the sixth dose at week 6 is about 40 mg/day, the seventh dose at week 7 is about 80 mg/day, and the eighth dose at week 8 and beyond is about 160 mg/day.

24. The method of claim 19, wherein the period of weekly ramp-up schedule administration lasts for five, six, seven, eight, nine or ten weeks, preferably five, six, seven, eight, or nine weeks.

25. The method of claim 1, wherein the B-cell malignancy is selected from chronic lymphocytic leukemia/small lymphocytic lymphoma (CLL/SLL) with low-tumor-burden, CLL/SLL with high-tumor-burden, or CLL/SLL with prior venetoclax treatment.

26. The method of claim 1, wherein the B-cell malignancy is mantle cell lymphoma (MCL).

27. The method of claim 1, wherein the B-cell malignancy is Waldenstrom macroglobulinemia (WM).

28. The method of claim 2, wherein (S)-7-(1-acryloylpiperidin-4-yl)-2-(4-phenoxyphenyl)-4,5,6,7-tetra-hydropyrazolo[1,5-a]pyrimidine-3-carboxamide (Compound B) is orally administrated at a dose 320 mg/day (preferably 160 mg twice daily or 320 mg once daily), and the Bcl-2 inhibitor is orally administrated by a weekly ramp-up schedule.

29. The method of claim 28, wherein (S)-7-(1-acryloylpiperidin-4-yl)-2-(4-phenoxyphenyl)-4,5,6,7-tetra-hydropyrazolo[1,5-a]pyrimidine-3-carboxamide (Compound B) is orally administrated beginning 8-12 weeks before Compound 1 is administrated.

30. The method of claim 28, wherein the weekly ramp-up schedule comprises the first dose at week 1, the second dose at week 2, the third dose at week 3, the fourth dose at week 4, the fifth dose at week 5, a subsequently weekly ramp-up schedule, and a recommended dose at a certain week and beyond, wherein the dose at the subsequent week is at least double of the dose the previous week until the weekly recommended dose has been met, and the subsequently weekly ramp-up schedule is a weekly ramp-up dosing schedule for 0, 1, 2, 3, or 4 weeks.

31. The method of claim 28, the Bcl-2 inhibitor is orally administrated at a dose according to a weekly ramp-up schedule beginning with 1 mg daily at week 1, wherein the dose at the subsequent week is at least double of the dose the previous week until the weekly recommended dose has been met, and the recommended dose is 40 mg, 80 mg, 160 mg, 320 mg, or 640 mg daily.

32. The method of claim 28, wherein the Bcl-2 inhibitor is orally administrated by weekly ramp-up schedule comprising a dose step of 1 mg, 2 mg, 5 mg, 10 mg, 20 mg, 40 mg, 80 mg, 160 mg, 320 mg or 640 mg daily.

33. The method of claim 28, wherein the first dose at week 1 is about 1 mg/day, the second dose at week 2 is about 2 mg/day, the third dose at week 3 is about 5 mg/day, the fourth dose at week 4 is about 10 mg/day, the fifth dose at week 5 is about 20 mg/day, the sixth dose at week 6 is about 40 mg/day, and the seventh dose at week 7 and beyond is about 80 mg/day.

34. The method of claim 28, wherein the first dose at week 1 is about 1 mg/day, the second dose at week 2 is about 2 mg/day the third dose at week 3 is about 5 mg/day, the fourth dose at week 4 is about 10 mg/day, the fifth dose at week 5 is about 20 mg/day, the sixth dose at week 6 is about 40 mg/day, the seventh dose at week 7 is about 80 mg/day, and the eighth dose at week 8 and beyond is about 160 mg/day.

35. The method of claim 28, wherein the period of weekly ramp-up schedule administration lasts for five, six, seven, eight or nine weeks.

36. The method of claim 2, wherein the B-cell malignancy is R/R CLL/SLL or nave CLL/SLL.

37. The method of claim 2, wherein the B-cell malignancy is MCL.

38. The method of claim 1 or 2, wherein the Bcl-2 is orally administrated once daily (QD).

39. The method of claim 1 or 2, wherein the B-cell malignancy has Bcl-2 expression.

40. The method of claim 1 or 2, wherein the B-cell malignancy has Bcl-2 Gly101Val mutation expression.

41. The method of claim 19 or 28, wherein the first dose at week 1 is about 1 mg/day, the second dose at week 2 is about 2 mg/day, the third dose at week 3 is about 5 mg/day, the fourth dose at week 4 is about 10 mg/day, the fifth dose at week 5 is about 20 mg/day, the sixth dose at week 6 is about 40 mg/day, the seventh dose at week 7 is about 80 mg/day, and the eighth dose at week 8 is about 160 mg/day, and the ninth dose at week 9 and beyond is about 320 mg/day.
